SEQUENCE LISTING (47 sequences)

SEQ ID NO: 1 - DNA, 23 bp, Artificial Sequence
  Description of Artificial Sequence Synthetic oligonucleotide
cttcctcttgtgctcttgctggg

SEQ ID NO: 2 - DNA, 23 bp, Artificial Sequence
  Description of Artificial Sequence Synthetic oligonucleotide
gccccagcaagagcaccaagagg

SEQ ID NO: 3 - DNA, 33 bp, Artificial Sequence
  Description of Artificial Sequence Synthetic oligonucleotide
acactctttccctacacgacgctcttccgatct

SEQ ID NO: 4 - DNA, 32 bp, Artificial Sequence
  Description of Artificial Sequence Synthetic oligonucleotide
gactggagttcagacgtgtgctcttccgatct

SEQ ID NO: 5 - DNA, 25 bp, Artificial Sequence
  Description of Artificial Sequence Synthetic oligonucleotide
gtcggaaaggcaagggcattcagca

SEQ ID NO: 6 - DNA, 25 bp, Artificial Sequence
  Description of Artificial Sequence Synthetic oligonucleotide
aaactgctgaatgcccttgcctttc

SEQ ID NO: 7 - DNA, 24 bp, Artificial Sequence
  Description of Artificial Sequence Synthetic oligonucleotide
gtcggccattgagcagtcgcatcc

SEQ ID NO: 8 - DNA, 24 bp, Artificial Sequence
  Description of Artificial Sequence Synthetic oligonucleotide
aaacggatgcgactgctcaatggc

SEQ ID NO: 9 - DNA, 25 bp, Artificial Sequence
  Description of Artificial Sequence Synthetic oligonucleotide
gtcgagtgtgaaaaatcccggcaat

SEQ ID NO: 10 - DNA, 25 bp, Artificial Sequence
  Description of Artificial Sequence Synthetic oligonucleotide
aaacattgccgggatttttcacact

SEQ ID NO: 11 - DNA, 24 bp, Artificial Sequence
  Description of Artificial Sequence Synthetic primer
ctgctcattgcacttatctacaag

SEQ ID NO: 12 - DNA, 26 bp, Artificial Sequence
  Description of Artificial Sequence Synthetic primer
gcaaattaaaatgttactcgcatctc

SEQ ID NO: 13 - DNA, 21 bp, Artificial Sequence
  Description of Artificial Sequence Synthetic primer
gctggtcaaccggacacgcgg

SEQ ID NO: 14 - DNA, 25 bp, Artificial Sequence
  Description of Artificial Sequence Synthetic primer
ctcgctgccgataggtcagatgtcg

SEQ ID NO: 15 - DNA, 22 bp, Artificial Sequence
  Description of Artificial Sequence Synthetic primer
gtgtgaaaaatcccggcaatgg

SEQ ID NO: 16 - DNA, 24 bp, Artificial Sequence
  Description of Artificial Sequence Synthetic primer
agggagccgataaagaggtcatcc

SEQ ID NO: 17 - DNA, 54 bp, Artificial Sequence
  Description of Artificial Sequence Synthetic primer
acactctttccctacacgacgctcttccgatctccaatttgaaactcagtttgc

SEQ ID NO: 18 - DNA, 51 bp, Artificial Sequence
  Description of Artificial Sequence Synthetic primer
gactggagttcagacgtgtgctcttccgatctgtcatcctgctggacatag

SEQ ID NO: 19 - DNA, 57 bp, Artificial Sequence
  Description of Artificial Sequence Synthetic primer
acactctttccctacacgacgctcttccgatctgcgcccaggaaacatttgctcaag

SEQ ID NO: 20 - DNA, 55 bp, Artificial Sequence
  Description of Artificial Sequence Synthetic primer
gactggagttcagacgtgtgctcttccgatctcgctgccgataggtcagatgtcg

SEQ ID NO: 21 - protein, 9 aa, Unknown
  Description of Unknown LAGLIDADG family peptide sequence
LAGLIDADG

SEQ ID NO: 22 - DNA, 16 bp, Artificial Sequence
  Description of Artificial Sequence Synthetic oligonucleotide
ggcaatgggccaagag

SEQ ID NO: 23 - DNA, 63 bp, Artificial Sequence
  Description of Artificial Sequence Synthetic oligonucleotide
ctgctgaatgcccttgcctttcgatcgccgcagggcatccaagtatcgccatccgggatgcga

SEQ ID NO: 24 - DNA, 51 bp, Artificial Sequence
  Description of Artificial Sequence Synthetic oligonucleotide
ctggcctttcgatcgccgcagggcatccaagtatcgccatccgggatgcga

SEQ ID NO: 25 - DNA, 64 bp, Artificial Sequence
  Description of Artificial Sequence Synthetic oligonucleotide
ctgctgaatgcccttgcctttcgatcgccgcagggcatccaagtatcgccatccgggactgcga

SEQ ID NO: 26 - DNA, 9 bp, Artificial Sequence
  Description of Artificial Sequence Synthetic oligonucleotide
ggccaagag

SEQ ID NO: 27 - DNA, 60 bp, Artificial Sequence
  Description of Artificial Sequence Synthetic oligonucleotide
ctgctgaatgcccttgcctttcgatcgccgcagatccaagtatcgccatccgggatgcga

SEQ ID NO: 28 - DNA, 55 bp, Artificial Sequence
  Description of Artificial Sequence Synthetic oligonucleotide
ctgctgaatgcccttgcctttcgatcgccgccaagtatcgccatccgggatgcga

SEQ ID NO: 29 - DNA, 68 bp, Unknown
  Description of Unknown CS1 sequence
gaaagacgaccctgctgaatgcccttgcctttcgatcgccgcagggcatccaagtatcgccatccggg

SEQ ID NO: 30 - DNA, 56 bp, Artificial Sequence
  Description of Artificial Sequence Synthetic oligonucleotide
gaaagacgacccttgcctttcgatcgccgcagggcatccaagtatcgccatccggg

SEQ ID NO: 31 - DNA, 65 bp, Artificial Sequence
  Description of Artificial Sequence Synthetic oligonucleotide
gaaagacgaccctgctgaatgcccttgcctttcgatcgccgcagatccaagtatcgccatccggg

SEQ ID NO: 32 - DNA, 68 bp, Artificial Sequence
  Description of Artificial Sequence Synthetic oligonucleotide
gaaagacgaccctggtctttgcacctgcctttggattcccgctaatcccatcctcgatgccactgatg

SEQ ID NO: 33 - DNA, 66 bp, Artificial Sequence
  Description of Artificial Sequence Synthetic oligonucleotide
gaaagacgaccctggtgaatgcccttgcctttcgatcgccgcagatccaagtatcgccatccggga

SEQ ID NO: 34 - DNA, 66 bp, Artificial Sequence
  Description of Artificial Sequence Synthetic oligonucleotide
gaaagacgaccctgctgaatgcccttgcctttcgatcgccgcagatccaagtatcgccatccggga

SEQ ID NO: 35 - DNA, 22 bp, Artificial Sequence
  Description of Artificial Sequence Synthetic oligonucleotide
gaccctggtctttgcacctgcc

SEQ ID NO: 36 - DNA, 22 bp, Artificial Sequence
  Description of Artificial Sequence Synthetic oligonucleotide
gaccctggtgaatgcccttgcc

SEQ ID NO: 37 - DNA, 22 bp, Artificial Sequence
  Description of Artificial Sequence Synthetic oligonucleotide
gaccctgctgaatgcccttgcc

SEQ ID NO: 38 - DNA, 54 bp, Unknown
  Description of Unknown CS1 sequence
accctgctgaatgcccttgcctttcgatcgccgcagggcatccaagtatcgcca

SEQ ID NO: 39 - DNA, 42 bp, Artificial Sequence
  Description of Artificial Sequence Synthetic oligonucleotide
acccttgcctttcgatcgccgcagggcatccaagtatcgcca

SEQ ID NO: 40 - DNA, 41 bp, Artificial Sequence
  Description of Artificial Sequence Synthetic oligonucleotide
  modified_base (41)..(41): a, c, t, g, unknown or other
acccttgcctttcgatcgccgcaggcatccatgtacctccn

SEQ ID NO: 41 - DNA, 42 bp, Artificial Sequence
  Description of Artificial Sequence Synthetic oligonucleotide
  modified_base (18)..(18): a, c, t, g, unknown or other
acccttgcctttccaccnccccagggcatcccagtatcgccc

SEQ ID NO: 42 - DNA, 34 bp, Unknown
  Description of Unknown WT sequence used for gRNA design
ccaggtagttacagtctagcagtactgatcatcg

SEQ ID NO: 43 - DNA, 34 bp, Artificial Sequence
  Description of Artificial Sequence Synthetic oligonucleotide
ccaggtagttacagtctagcagtacggatcatcg

SEQ ID NO: 44 - DNA, 34 bp, Unknown; identical to SEQ ID NO: 43
  Description of Unknown WT sequence used for gRNA design

SEQ ID NO: 45 - DNA, 34 bp, Artificial Sequence
  Description of Artificial Sequence Synthetic oligonucleotide
ccaggtagttacagtctagctgtacggatcatcg

SEQ ID NO: 46 - DNA, 41 bp, Unknown
  Description of Unknown WT sequence used for gRNA design
ttagccgccaggtagttacagtctagcagtacgcatcatcg

SEQ ID NO: 47 - DNA, 41 bp, Artificial Sequence
  Description of Artificial Sequence Synthetic oligonucleotide
ttagacgccaggtagttacagtctagcaatacggatcatcg

(19) United States
(12) Patent Application Publication
(10) Pub. No.: US 2022/0325338 A1
(43) Pub. Date: Oct. 13, 2022

(54) SYSTEMS AND METHODS FOR PERFORMING AND MEASURING HOMOLOGOUS CHROMOSOME TEMPLATE REPAIR

(71) Applicant: The Regents of the University of California, Oakland, CA (US)

(72) Inventors: Ethan Bier, San Diego, CA (US); Annabel Guichard, La Jolla, CA (US); Zhiqian Li, San Diego, CA (US)

(21) Appl. No.: 17/711,793

(22) Filed: Apr. 1, 2022

Related U.S. Application Data
(60) Provisional application No. 63/169,988, filed on Apr. 2, 2021.

(51) Int. Cl. C12Q 1/6858; C12N 9/22; C12N 15/11; C12N 15/85; C12Q 1/6897; C12N 15/90

(52) U.S. Cl. CPC C12Q 1/6858; C12N 9/22; C12N 15/11; C12N 15/8509; C12Q 1/6897; C12N 15/907; C12N 2310/20; C12N 2800/80; C12N 2800/105; C12N 2015/859

(57) ABSTRACT

Provided herein are systems and methods for performing repair of mutant chromosomes in cells by using the homologous chromosome as a template for homology directed repair. Also provided herein are CopyCatcher systems, methods, and organisms for the study and measurement of homologous chromosome template repair and related mechanisms in cells and organisms.

### CROSS REFERENCE

This application claims the benefit of U.S. Provisional Application No. 63/169,988 filed Apr. 2, 2021, which application is incorporated herein by reference in its entirety

### GOVERNMENT SPONSORSHIP

This invention was made with government support under grant No. GM117321 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Mar. 31, 2020 is named 24978-0710_SL.txt and is 11,847 bytes in size.

### TECHNICAL FIELD

The present invention relates to versatile active genetic elements for detecting and quantifying interhomolog somatic gene conversion. The present invention also relates to methods of repairing mutant chromosomes using a homologous chromosome as a repair template.

### BACKGROUND

CRISPR-based active genetic elements are self-propagating cassettes carrying gRNAs (±Cas9 or associated cargos) that cut the genome at the location where those elements are inserted¹. Active genetic elements, particularly gene-drives (carrying linked gRNA and Cas9 transgenes), offer great potential for population modification or suppression by repairing double-stranded breaks (DSBs) through homology directed repair (HDR) in germline cells to disseminate beneficial genetic cargos throughout an insect population or to decrease (suppress) the population size^{1,2,3}. Alternatively, DSBs can be repaired through the error-prone non-homologous end-joining (NHEJ) pathway, by ligating the two broken DNA ends together or creating indels at the cut site when challenged by repeated Cas9 cleavage. NHEJ is active throughout the cell cycle, rather than being confined to late S and G2 phases as is HDR. NHEJ is thus considered to be the primary DSB repair pathway in somatic cells under normal circumstances⁴⁻¹⁰.

While CRISPR-based gene-drive elements can be highly efficient in copying themselves in germline cell lineages, the general view has been that CRISPR induced breaks in somatic cells are repaired predominantly by the NHEJ pathway^{1,2,7,11-15}, and that if cleavage-resistant mutations generated by imprecise NHEJ repair arise during early embryonic stages (prior to allocation of the germline), they reduce subsequent drive efficiency in the germline^{2,16-23}. This interpretation of NHEJ alleles limiting gene-drive performance is also consistent with the lower efficiency of HDR relative to NHEJ typically observed in cultured mammalian cells^{7,24}.

When engaging the HDR pathway, somatic cells maintain genome integrity by employing identical sister chromatids as DSB repair templates. This post-replicative and restorative function of HDR in somatic cells contrasts with its role in the germline where meiotic factors promote DSB-dependent recombination between homologous chromosomes^{25,26}. Also, dysfunctional engagement of HDR in which the homologous chromosome rather than the sister chromatid serves as the repair template results in loss-of-heterozygosity (LOH) phenotypes that can lead to oncogenic outcomes and developmental defects^{27,28}. Collectively, these considerations support the current hypothesis that somatic cells employ either NHEJ or sister chromatid based HDR as the predominant DSB repair strategies, providing a ready explanation for the relatively low rates of precise gene editing typically achieved when using exogenously provided DNA templates.

Although DSB repair mechanisms differ between germline and somatic cells in several important respects, a set of core factors play essential roles in both repair processes including: Ku70/Ku80 heterodimer²⁹, RAD 51³⁰, MRE11^{29,31}, CtIP³²⁻³⁴ and 53BP1^{35,36}. How other cellular processes, including chromosome pairing and remodeling, might influence DSB repair, particularly in somatic cells, remains largely unknown.

### SUMMARY OF THE INVENTION

The present invention provides CRISPR-based active genetic CopyCatcher systems to detect and quantify genetic somatic gene conversion (SGC) events in vivo in Drosophila. CopyCatchers reveal unexpectedly high rates of SGC in Drosophila and can be employed as versatile tools for identifying genetic components required for the SGC process. Homolog-templated SGC can also take place in mammalian cells, and loci impacting the rates of such repair (e.g., c-MYC) function in a conserved fashion in this process. Collectively, these results suggest that CopyCatchers offer novel efficient systems for tracking and dissecting homolog-based copying mechanisms in somatic cells and offer a new potential avenue for pursuing precise human gene therapy.

In another aspect of the invention, Homologous Chromosome-Templated Repair (HTR) methods provided herein are conceived to correct a wide range of disease-causing mutations using functional genetic information provided by the homologous chromosome instead of a donor plasmid. This strategy involves the design of a guide RNA that selectively cuts the mutant chromosome at or near the site of the mutation, but not the wild-type allele present on the homologous chromosome. Such a selective guide RNA would be delivered along with the Cas9 endonuclease (or similar enzyme or variant, such as D10A or H840A nickases) into mutant tissues (via liposomes or polyplexes) to correct the mutant allele. Alternatively, DNA encoding these components (guide RNA+Cas9) can be delivered using viral vectors.

Once the targeted allele is cleaved, homology-directed repair (HDR) using the homologous chromosome as corrective template (instead of the identical sister chromatid or a donor plasmid) can then result in correction of the mutant allele with the endogenous functional sequence (e.g., a wild-type allele). This method can be applicable to dominant heterozygous disease-causing mutations, as well as to recessive mutations in trans-heterozygous patients, carrying two different mutations in the same gene.

An exemplary illustration of an HTR method provided herein compared to a conventional CRISPR-Cas9 approach is shown in FIG. 10. In a conventional approach, the CRISPR-Cas9 system initiates a double stranded break in both chromosomes (mutant and healthy) of a cell. This double stranded break is then repaired using homology directed repair with a repair template (e.g., a donor plasmid). This approach creates a risk that one or both of the chromosomes results in additional mutations owing to potential non-homologous end joining (NHEJ) repair of the double stranded breaks. Conversely, the HTR methods provided herein cleave only the mutant chromosome specifically targeted by the gRNA, thus eliminating the risk of NHEJ induced mutations into the healthy chromosome. Additionally, no exogenous donor plasmid is required for the repair of the mutant allele, simplifying the Cas9/gRNA delivery process and resulting in two copies of the wild type allele.

The invention can be used in methods for gene therapy to correct dominant mutations in heterozygous patients, for example but not limited to: Autosomal Dominant Polycystic Kidney Disease (ADPKD), Familial Hypercholesterolemia, Hemorrhagic Telangiectasia, Hereditary Spherocytosis, Marfan's Syndrome, Neurofibromatosis, congenital (papular) atrichia, and Cadasil syndrome. The invention can be used in methods for gene therapy to correct trans-heterozygous combinations of recessive mutations (i.e., patient carries two different non-functional mutations in the same gene), for example but not limited to: Cystic fibrosis, haemochromatosis, and retinitis pigmentosa. The invention can be used in methods to alter histocompatibility genotypes to make patients competent recipients for a broader range of transplantations or blood transfusions.

The HTR method takes advantage of the endogenous DNA repair machinery that cells use to correct double stranded DNA breaks using the homolog chromosome as a repair template. This scarless repair in vivo process as quantified rigorously in Drosophila (30-50% of cells) is far more efficient than existing in vitro HDR-methods based on the introduction of an exogenous plasmid DNA template (1-5%), which are notoriously inefficient and inapplicable for in vivo correction.

The HTR method results in restoration of endogenous gene sequence and function by converting the mutant allele to a normal functional form of the gene. The repaired gene will retain its native structure and its expression will rely on endogenous control sequences.

HTR relies on providing minimal genetic information (only gRNA and Cas9, or DNA encoding these components). This also should result in a more efficient delivery of therapeutic components, as no introduction of large donor plasmid DNA template is required.

The HTR method is based on selective cutting of the mutant allele of a gene. A gRNA must be designed that selectively targets the mutant allele of a gene, but not the functional sequence present on the homologous chromosome in a heterozygous patient (FIG. 12). Delivery of such gRNA to relevant tissues along with a source of Cas9 will result in DNA cleavage of the mutant allele only. Homology directed repair (HDR) using the homologous chromosome as template will correct the mutation, resulting in restoration of a functional endogenous copy of the gene residing in its normal location with its natural structure (e.g., intron-exon structure) and regulation. When the sister chromatid is being used as template, the original mutant sequence will be regenerated, which can then be acted on again until corrected from the homologous chromosome. In some cases, NHEJ will result in a second mutation that cannot be acted on again by the same guide RNA, but such events would not aggravate the subject's condition, since that allele was already non-functional. This scarless genetic replacement provides a fully active copy of the gene and thereby restore normal function.

The two major scenarios for such HTR mediated repair are shown in FIG. 11. First, when a patient carries a dominant mutant allele on one chromosome and a functional copy on the homologous chromosome, this would restore two functional copies of the gene. Second, when a patient carries two different recessive mutations in the same gene (optimally spaced apart by ˜1 kb or more) wherein correction of either mutation with functional sequences from the wild-type homolog gene can restore one functional copy of gene activity, functionally converting the patient to approximately carrier status with disease symptoms reduced or eliminated.

In one aspect, provided herein, is a homologous chromosome template repair (HTR) system, comprising a gene editing system configured to: a) cut a mutant allele of a cell at or near a mutation in the mutant allele but not cut a corresponding homologous allele without the mutation; and b) allow the corresponding homologous allele to act as a template for homology directed repair of the mutant allele, thereby repairing the mutation in the mutant allele.

In some embodiments, the gene editing system comprises a guide RNA and an endonuclease enzyme. In some embodiments, the endonuclease is selected from a meganuclease, a Transcription Activator Like Effector Nucleases (TALEN), a Zinc-Finger Nucleases (ZFN), and a Clustered Regularly Interspaced Short Palindromic Repeats (CRISPR)-associated system (Cas), and derivatives thereof. In some embodiments, the endonuclease is a Cas or a derivative thereof. In some embodiments, the endonuclease is a Cas9 or a derivative thereof. In some embodiments, the endonuclease is a Cas9 variant selected from D10A and H840A.

In some embodiments, the gene editing system is configured to perform a single-stranded cut of the mutant allele. In some embodiments, he single-stranded cut is in the encoding strand of the mutant allele. In some embodiments, the single-stranded cut is in the template strand of the mutant allele.

In some embodiments, the mutation in the mutant allele creates an endonuclease recognition site in the mutant allele. In some embodiments, he endonuclease recognition site is a protospacer adjacent motif (PAM). In some embodiments, the mutation in the mutant allele is near an endonuclease recognition site present on both the mutant allele and the homologous allele and wherein the gene editing system is configured to cut the mutant allele at the mutation. In some embodiments, the mutation in the mutant allele is near a polymorphic site near an endonuclease recognition site and wherein the gene editing system is configured to cut the mutant allele at the polymorphic site. In some embodiments, the mutation is a substitution of one or more nucleotides, an insertion of one or more nucleotides, a deletion of one or more nucleotides, or any combination thereof. In some embodiments, the system further comprises comprising an agent which modulates the activity or expression of a protein implicated in homology directed repair or non-homologous end joining.

In some embodiments, the cell is a eukaryotic cell. In some embodiments, the cell is an insect cell. In some embodiments, the cell is a mammalian cell. In some embodiments, the cell is a human cell. In some embodiments, the cell is a cell of a multicellular organism. In some embodiments, the multicellular organism is an insect. In some embodiments, the multicellular organism is a mammal. In some embodiments, the multicellular organism is a human. In some embodiments, the cell is a somatic cell of the organism. In some embodiments, the gene editing system does not comprises an exogenous repair template.

Also provided herein is a vector encoding an HTR system as provided herein. Also provided herein is a method of repairing a mutation in a cell comprising introducing into the cell an HTR system or a vector encoding an HTR system as provided herein. Further provided herein is a method of performing gene therapy in a subject comprising administering to the subject an HTR system or a vector encoding an HTR system as provided herein. In another aspect herein is a pharmaceutical composition comprising a vector encoding an HTR system and a pharmaceutically acceptable carrier.

In another aspect, provided herein, is a method of performing a homologous chromosome template repair (HTR) in a cell, comprising: a) contacting a mutant allele with a gene editing system configured to cut the mutant allele at or near a mutation in the mutant allele but not to cut a homologous allele without the mutation; b) cutting the mutant allele at or near the mutation in the mutant allele; c) using the homologous allele as a template for homology directed repair (HDR); and repairing the mutation in the mutant allele.

In some embodiments, the gene editing system comprises a guide RNA and an endonuclease enzyme. In some embodiments, the endonuclease is selected from a meganuclease, a Transcription Activator Like Effector Nucleases (TALEN), a Zinc-Finger Nucleases (ZFN), and a Clustered Regularly Interspaced Short Palindromic Repeats (CRISPR)-associated system (Cas), and derivatives thereof. In some embodiments, the endonuclease is a Cas or a derivative thereof. In some embodiments, the endonuclease is a Cas9 or a derivative thereof. In some embodiments, the endonuclease is a Cas9 variant selected from D10A and H840A.

In some embodiments, the guide RNA is complementary to a portion of the mutant allele but not the homologous allele. In some embodiments, the gene editing system is configured to perform a single-stranded cut of the mutant allele. In some embodiments, the single-stranded cut is in the encoding strand of the mutant allele. In some embodiments, the single-stranded cut is in the template strand of the mutant allele.

In some embodiments, the method comprises introducing the gene editing system or a vector encoding the gene editing system into the target cell. In some embodiments, the vector is a plasmid or a viral vector.

In some embodiments, the mutation in the mutant allele creates an endonuclease recognition site in the mutant allele. In some embodiments, the endonuclease recognition site is a protospacer adjacent motif (PAM). In some embodiments, the mutation in the mutant allele is near an endonuclease recognition site present on both the mutant allele and the homologous allele and wherein the gene editing system is configured to cut the mutant allele at the mutation. In some embodiments, the mutation in the mutant allele is near a polymorphic site near an endonuclease recognition site and wherein the gene editing system is configured to cut the mutant allele at the polymorphic site. In some embodiments, the mutation is a substitution of one or more nucleotides, an insertion of one or more nucleotides, a deletion of one or more nucleotides, or any combination thereof. In some embodiments, the method further comprises introducing into the cell an agent which modulates the activity or expression of a protein implicated in homology directed repair or non-homologous end joining.

In some embodiments, the cell is a eukaryotic cell. In some embodiments, the cell is an insect cell. In some embodiments, the cell is a mammalian cell. In some embodiments, the cell is a human cell. In some embodiments, the cell is a somatic cell. In some embodiments, the cell is a cell of a multicellular organism. In some embodiments, the multicellular organism is an insect. In some embodiments, the multicellular organism is a mammal. In some embodiments, the multicellular organism is a human.

Also provided herein in an aspect is an engineered cell for investigating homologous chromosome directed repair, comprising: a) a first allele which does not express an encoded gene; b) a second allele homologous to the first allele, wherein the second allele comprises a mutation relative to the first allele; c) a guide RNA configured to recruit an endonuclease enzyme to facilitate a cut in the second allele at or near the mutation but not cut the first allele; wherein the system is configured such that homology directed repair (HDR) of the second allele with the first allele as a template after a cut by the endonuclease enzyme results in the second allele encoding the encoded gene.

In some embodiments, the encoded gene comprises a reporter gene. In some embodiments, the reporter gene encodes a fluorescent protein or a bioluminescent protein. In some embodiments, the reporter gene is preceded on the first allele by a sequence encoding a self-cleavage peptide. In some embodiments, the reporter gene is comprised in an intron of a native gene of the first allele. In some embodiments, the reporter gene is preceded on the first allele by a splice acceptor. In some embodiments, the HDR results in the reporter gene being incorporated into the second allele. In some embodiments, the encoded gene comprises a native gene of the first allele.

In some embodiments, the mutation of the second allele results in an altered phenotype in the organism relative to an organism which expresses the native gene without the mutation. In some embodiments, the HDR results in a change in phenotype in the organism relative to the organism with the mutation. In some embodiments, the first allele is modified to not express the encoded gene by a mutation of or a deletion of a start codon. In some embodiments, the mutation in the second allele comprises a nucleotide substitution, nucleotide addition, or nucleotide deletion. In some embodiments, the second allele does not encode the encoded gene of the first allele prior to homology directed repair using the first allele as a template.

In some embodiments, the guide RNA is encoded in the first allele. In some embodiments, the guide RNA is encoded in an intron a native gene of the first allele. In some embodiments, the endonuclease is selected from a meganuclease, a Transcription Activator Like Effector Nucleases (TALEN), a Zinc-Finger Nucleases (ZFN), and a Clustered Regularly Interspaced Short Palindromic Repeats (CRISPR)-associated system (Cas), and derivatives thereof. In some embodiments, the endonuclease is a CRISPR associated system (Cas) or a derivative thereof. In some embodiments, the endonuclease is Cas9 or a derivative thereof. In some embodiments, the endonuclease is a Cas9 variant selected from D10A and H840A. In some embodiments, the endonuclease is configured to perform a single-stranded cut of the second allele. In some embodiments, the engineered cell comprises the endonuclease or a vector encoding the endonuclease.

In some embodiments, the cell is a eukaryotic cell. In some embodiments, the cell is a cell of a multicellular organism. In some embodiments, the organism is an insect. In some embodiments, the organism is a Drosophila melanogaster. In some embodiments, the organism is a mammal or a mammalian cell.

In a further aspect provided herein is a method for investigating homologous chromosome template repair in an organism, comprising: a) introducing into a first allele of the organism: i) a first polynucleotide sequence comprising: 1) a reporter gene; or 2) at least a portion of a native gene or a mutation of the native gene which results in a change in phenotype in the organism when the portion of the native gene or the mutation of the native gene is incorporated into a second allele homologous to the first allele by homology directed repair; and ii) a second polynucleotide sequence encoding a guide RNA configured to recruit an endonuclease enzyme to facilitate a cut in the second allele at or near a mutation but not cut the first allele; b) introducing the endonuclease or a vector encoding the endonuclease into the organism or a descendant of the organism; and c) performing homology directed repair of the second allele with the first allele as a template.

In some embodiments, the method further comprises a step of measuring expression of the reporter gene or the change in phenotype in the organism or the descendant of the organism. In some embodiments, the method further comprises introducing into the organism or the descendant of the organism an agent configured to modulate the expression or activity of an additional gene. In some embodiments, the agent is interfering RNA (RNAi) or a vector encoding RNAi. In some embodiments, the additional gene is implicated or associated with DNA repair. In some embodiments, the method further comprises a step of comparing expression of the reporter gene or the change in phenotype in the organism or the descendant of the organism and comparing the expression or change in phenotype to a reference organism which did not receive the agent.

In some embodiments, the first allele does not express the first polynucleotide sequence. In some embodiments, the first allele comprises a mutation or deletion of a start codon of the first allele. In some embodiments, the reporter gene encodes a fluorescent protein or a bioluminescent protein. In some embodiments, the reporter gene is preceded on the first allele by a sequence encoding a self-cleavage peptide. In some embodiments, the reporter gene is comprised in an intron of a native gene of the first allele. In some embodiments, the reporter gene is preceded on the first allele by a splice acceptor. In some embodiments, the second allele does not encode the encoded gene of the first allele prior to homology directed repair using the first allele as a template.

In some embodiments, the endonuclease is selected from a meganuclease, a Transcription Activator Like Effector Nucleases (TALEN), a Zinc-Finger Nucleases (ZFN), and a Clustered Regularly Interspaced Short Palindromic Repeats (CRISPR)-associated system (Cas), and derivatives thereof. In some embodiments, the endonuclease is a CRISPR associated system or a derivative thereof. In some embodiments, the endonuclease is CRISPR-Cas9 or a derivative thereof. In some embodiments, the endonuclease is a Cas9 variant selected from D10A and H840A. In some embodiments, the endonuclease is configured to perform a single-stranded cut of the second allele. In some embodiments, the mutation in the second allele comprises a nucleotide substitution, nucleotide addition, or nucleotide deletion.

In some embodiments, the organism is a eukaryotic organism. In some embodiments, the organism is a multicellular organism. In some embodiments, the organism is an insect. In some embodiments, the organism is a Drosophila melanogaster. In some embodiments, the organism is a mammal or a mammalian cell.

### BRIEF DESCRIPTION OF THE DRAWINGS

FIG. 1A shows a schematic representation of a CopyCatcher system, including light gray boxes on donor chromosome marked Exon1 and Exon2: exons of targeted gene on the donor chromosome; dark gray boxes on receiver chromosome marked Exon1 and Exon 2: exons on the receiver chromosome; black lines: genomic DNA; dark black box: splice acceptor site (SA); light gray box: T2A self-cleavage peptide; medium gray arrow: DsRed reporter; dark gray right pointing arrow: gRNA; medium gray left pointing arrow: selection marker mCerulean; hourglass marks: point mutations at or around the ATG codons of the endogenous gene; short black line near scissors: Cas9/gRNA cleavage site; light gray circle and wavy dark lines: Cas9/gRNA complex. Scissors in a denote insertion of cargo cassettes into DSB on homologous chromosome including SA, T2A, gRNA and selection marker mCerulean. “Copying,” “No cutting,” and “NHEJ” schemes show distinct outcomes of three DSB repair mechanisms followed by Cas9/gRNA cleavage of the homologous “receiver” chromosome. Slashes on the second exons in “Copying,” “No cutting,” and “NHEJ” schemes indicate loss-of-function on the marked chromosomes. Labels indicate resulting phenotypes.

FIG. 1B shows mosaic clones of somatic gene conversion (SGC) in a CopyCatcher line. Photographs show the phenotypes and fluorescence patterns of the CopyCatcher elements inserted into the intron of white (w^{[ATG-,3XP3-CC]}) loci in flies without or with associated ATG⁻ point mutations, or F1 mosaics resulting from Cas9-mediated copying. Right most panel is a higher magnification view of area delineated by white box in the lower magnification views immediately to the left.

FIG. 1C shows mosaic clones of somatic gene conversion (SGC) in a CopyCatcher line. Photographs show the phenotypes and fluorescence patterns of the CopyCatcher element inserted into the intron of ple (ple^{[ATG-,CC]}) loci in flies without or with associated ATG point mutations, or F1 mosaics resulting from Cas9-mediated copying. SGC clones generated by ple^{[ATG-,CC]} are outlined with white dotted lines. Right most panels show higher magnification views of areas delineated by white boxes in the lower magnification views immediately to the left

FIG. 2A shows paternal and maternal crossing schemes using either F₀ males or females carrying Cas9 transgenes inserted in the yellow locus expressed by different promoters and marked with 3XP3-DsRed (in the eye). Trans-heterozygous F₁ females obtained in both crosses carrying both CopyCatcher elements and static Cas9 expression cassettes were used to score SGC.

FIG. 2B shows mosaic phenotypes of the F₁ trans-heterozygous y⁺,w^{[ATG-,CC]}/y^{[Cas9]}, w⁺ compound eyes.

FIG. 2C shows SGC rates measured by the fraction of F₁ female progeny having double-sided mosaic eyes. Each dot represents SGC averages from single vials. Asterisks represent significance: three asterisks (p<0.001), two asterisks (p<0.01), one asterisk (p<0.05), and ns (not significant). Error bars indicate mean±S.D.

FIG. 2D shows mosaic phenotypes of the F₁ trans-heterozygous y^{[Cas}9]/+; ple^{[ATG-,CC]}/+ thorax bristles. Clones of pale bristles delineated by dotted white lines denote the patches created by SGC events.

FIG. 2E shows SGC rates measured by the fraction of pale thorax bristles relative to the total thorax bristles in individual F₁ trans-heterozygous females. Each dot represents the percent of pale bristles for an individual fly (as shown in panel FIG. 2D). Asterisks represent significance: three asterisks (p<0.001), two asterisks (p<0.01), one asterisk (p<0.05), and ns (not significant). Error bars indicate mean±S.D.

FIG. 3A shows a workflow of genetic RNAi screen using ple^{[ATG-,CC]}.

FIG. 3B shows a heatmap displays the genetic screen results for genes modulating ple^{[ATG-,CC]} induced SGC. Values shown as fold change of SGC frequency by normalizing the pale bristles on each single fly to averaged control flies. The value is calculated by dividing the SGC frequency with knock-down indicated genes by averaged control SGC frequency that obtained with an shRNA targeting mCherry. Scores less than 1 indicate genes promoting SGC (black stars near the top of the DNA Pairing bracket and DSB Repair bracket indicate the top SGC promoters) while scores greater than 1 represent inhibitors of SGC (stars near the bottom of the DNA Pairing bracket and DSP Repair bracket indicate the top SGC inhibitors).

FIG. 3C shows examples of knocking down SGC inhibitor (Ku80) and promoter (fs(1)h) genes. The dominant SGC patches with light thoracic pigmentation and pale bristles were delineated with dotted white lines. White appearing spots inside the dotted white lines of the second column indicate locations where RFP fluorescence was observed.

FIG. 3D shows a validation of the top SGC modulating candidates in individuals carrying either heterozygous loss of function alleles or MS1096-GAL4 and UAS over-expression constructs. Each dot represents the relative fraction of pale bristles scored for a single fly. Asterisks represent significance: three asterisks (p<0.001), two asterisks (p<0.01), one asterisk (p<0.05), and ns (not significant). Error bars indicate mean±S.D.

FIG. 3E shows a key gene information for the top SGC modifiers identified by the ple^{[ATG-,CC]} CopyCatcher RNAi screen (FIG. 3B).

FIG. 4A shows a workflow for testing HDR rates resulting from RNAi knock-down of human orthologs of top hits modifying Drosophila SGC in human GAPHD-copGFP cells. Both exogenous plasmid and homologous chromosome templated somatic HDR were quantified with GFP fluorescent readout. Three biological replicates were conducted.

FIG. 4B shows FACS plots with donor plasmid mediated DSB repair. Each section stands for: Q₁: mCherry⁺ GFP⁻=HDR, Q₂: mCherry⁺ GFP⁺=none, Q3: mCherry⁻ GFP⁺=uncut, Q₄: mCherry⁻ GFP⁻=NHEJ. Averaged frequency of each events was labeled.

FIG. 4C shows a histogram of plasmid-mediated HDR frequency (light bars) and HDR/uncut ratio (dark bars) in GAPDH-copGFP heterozygous cell line with or without knock-down candidate SGC modifier homologs. Asterisks represent significance: four asterisks (p<0.0001), three asterisks (p<0.001), two asterisks (p<0.01), one asterisk (p<0.05), and ns (not significant). Error bars indicate mean±S.D.

FIG. 4D shows a table indicating HDR frequency templated with homologous chromosome with potential SGC modifiers knocking down.

FIG. 4E shows a histogram for FIG. 4D. Asterisks represent significance: four asterisks (p<0.0001), three asterisks (p<0.001), two asterisks (p<0.01), one asterisk (p<0.05), and ns (not significant). Error bars indicate mean±S.D.

FIG. 5A shows a system designed to visually evaluate allelic HTR at white in somatic tissues, depicting a transgenic gene-drive element (y^{ccw}) inserted in the yellow locus and encoding two guide RNAs, one targeting the yellow gene at the site of its insertion, and the other (white-gRNA), targeting Cas9-dependent cleavage of Cut-Sensitive loss-of-function (CS−) alleles created in the third exon of the white gene. Functional Cut-Resistant alleles (CR+) combined with an ATG− mutation (located ˜3.7 Kb upstream of the cut site) on the homologous chromosome may serve as template for allelic conversion. Successful repairs generate functional ATG+ CR+ alleles and produce visible red (white⁺) eye clones. The CS2− allele can also be repaired through random NHEJ mutations, while the CS1− can only be repaired through HDR. The Cas9 nuclease is provided from an autosomal source (vasaCas9), expressed in the germline and somatically.

FIG. 5B shows a DNA sequence of CS and CR alleles employed in this study. gRNA binding site and PAM site are labeled above the relevant sequences. The in-frame CS1− allele consists of a 12 nt deletion located 18 nt upstream of the cut site, and the frameshift CS2− allele consists of a 1 nt insertion, located 25 nt downstream of the cut site. The functional CR+ allele consists of a 3 nt deletion at the cut-site and is combined with an ATG− mutation (7 nt deletion covering the translation initiation site). The 1-o-f frameshift CR− allele consists of an 8 nt deletion at the cut site and was combined with the same ATG− mutation. Figure discloses SEQ ID NOS 22, 23, 22, 24, 22, 25, 26, 27, 26, and 28, respectively, in order of appearance.

FIG. 5C shows clonal phenotypes reveal HDR, NHEJ and combination repair events after Cas9-induced DSB of CS− alleles and no repair phenotype in the control flies lacking Cas9 (upper panels). y^{ccw} w^{ATG- CR+}/y⁺ w^{ATG+ CS1-} females expressing Cas9 show large solid red eye clones (corrective HTR). Cognate animals carrying an unfunctional CR− allele (y^{ccw} w^{ATG- CR-/ATG+ CS1-}, non-corrective HTR) do not show any red clones, demonstrating that the homologous allele provides template for corrective or non-corrective HTR to create ATG+ CR+ or ATG+ CR− alleles, respectively. In y^{ccw} w^{ATG+ CS1-}/Y; Cas9 males (which lack a homologous X chromosome), no red eye clones are visible (non-restorative NHEJ), showing that the 12 nt deletion CS1− allele cannot be repaired through mutagenic NHEJ processes. In contrast, y^{ccw} w^{ATG+ CS2-}/Y; Cas9 males show prevalent red clones (restorative NHEJ), consistent with the possibility that the 1 nt insertion CS2− allele can be repaired through frame restorative NHEJ. According to this expectation y^{ccw} w^{ATG- CR+/ATG+ CS2-} females (corrective HTR+NHEJ) show overall more red eye clones than y^{ccw} w^{ATG- CR+/ATG+ CS1-} females, as w⁺ clones derive from the CS2− alleles result from both NHEJ and HDR-mediated repairs. w^{ATG- CR-/ATG+ CS2-} females (restorative NHEJ) show fewer red clones as only NHEJ-dependent functional restoration may operate in these animals. Scale bar on eye picture here and in subsequent figures represents 100 um.

FIG. 5D shows quantification of HDR and NHEJ events using w⁺ clones visual grading. For each allelic combination, eyes were ranked on a 0-5 scale. Results from six eyes were averaged and plotted. y^{ccw} w^{ATG- CR+/ATG+ CS1-} and y^{ccw} w^{ATG- CR+/ATG+ CS2-} control flies have completely white eyes, consistent with CS1− and CS2− being null alleles. y^{ccw} w^{ATG- CR+/ATG+ CS1-}; Cas9 females show significant amounts of red clones, indicating that repair processes are Cas9-dependent. y^{ccw} w^{ATG- CR+/ATG+ CS2-} females show significantly more repair, consistent with red clones resulting exclusively from HDR repair in CS1− animals, and from HDR+NHEJ repair for the CS2− allele. y^{CCW} w^{ATG+ CS1-}/Y; Cas9 males show no visible repair, while y^{ccw} w^{ATG+ CS2-}/Y; Cas9 males show high levels of repair, which can only result from NHEJ processes. P-values for unpaired parametric t test analysis are indicated using standard symbolism here and in subsequent figures: p<0.0001=****; p<0.001=***; p<0.01=**; p<0.05=*. Bars denote mean value and standard deviation.

FIG. 6A shows clonal eye phenotypes reveal HDR, NHEJ and combination repair events after D10A-induced nicks, in animals carrying the white cut-sensitive ATG+ CS1− allele (top row) or the ATG+ CS2− allele (bottom row), combined with the y^{ccw} element and functional cut-resistant white ATG− CR+ allele (left panels) or the unfunctional ATG− CR− allele (middle panels), or the Y chromosome in males (right panels). Restored white⁺ function appears in a dense array of small red clones, also revealed by GFP fluorescence, which is blocked by eye pigments, but shines through unpigmented white− areas. Phenotypes reveal that homologous chromosome-dependent repair operates efficiently but late in development to restore white+ function in y^{ccw} w^{ATG- CR+/ATG+ CS1-}; D10A (corrective HTR) and w^{ATG- CR+/ATG+ CS2-}; D10A females, but not in y^{ccw} w^{ATG- CR-/ATG+ CS1-}; D10A (non-corrective HTR). In y^{ccw} w^{ATG- CR-/ATG+ CS2-}; D10A females, exceedingly low-level NHEJ-dependent repair is visible through minute white⁺ clones (restorative NHEJ). Similarly, y^{ccw} w^{ATG+ CS2-}; D10A males show low amounts of repair (restorative NHEJ, bottom right images), consistent with the low mutagenic activity of Nickases. In contrast, y^{ccw} w^{ATG+ CS1-}/Y D10A; cognates do not display any white+ clones (non-restorative NHEJ, top right images), consistent with our established observation that unlike CS2−, the CS1− allele cannot be repaired through NHEJ.

FIG. 6B shows global quantitative comparison of Cas9- and Nickase-induced allelic repair events by image analysis. Total pigmented surface area was calculated with image J and % correction was calculated as: (pigmented area)×100/(total surface of the eye). D10A-induced repair appears significantly higher (p<0.0001) than Cas9-induced repair for both CS1− (46% vs 22%) and CS2− alleles (66% vs 38%). In Cas9-expressing animals, repair of the CS2− allele (38%) is higher than for the CS1− allele (22%), as is expected from the contribution of the NHEJ pathway to CS2−, but not CS1−, repair. In males, high levels of NHEJ-only repair (˜31%) are observed in y^{ccw} w^{ATG+ CS2-}/Y; Cas9 males, contrasting with the low levels (1.5%) exhibited y^{ccw} w^{ATG+ CS2-}/Y; D10A males, consistent with D10A causing very few NHEJ mutations compared to Cas9.

FIG. 6C shows DNA sequence chromatograms quantitatively showing repair events for the CS1− allele. Reference w⁺ DNA sequence around the white-gRNA cut site is shown on top. Chromatograms for CS1−/CS1− and CR+/CR+ homozygous animals (1^{st} and 2^{nd} rows) show the 12 nt deletion and the 3 nt deletion, respectively. In CS1−/CR+ control animals (3^{rd} row), overlapping peaks of similar heights, start precisely at the breakpoint of the CS1− 12 nt deletion. Upon introduction of Cas9 (4^{th} row), peaks corresponding to donor sequences (CR+) appear consistently higher than peaks corresponding to the CS1− allele, reflecting on successful HTR. This effect is even more pronounced in D10A-expressing animals (5^{th} row), confirming that allelic repair operates more efficiently after SSB than DSB. Inset on the right shows an enlarged segment, and peaks used for the quantitative analysis in FIG. 6D (chosen for low distortion), are indicated with an asterisk. Figure discloses SEQ ID NOS 29-37, respectively, in order of appearance by graph.

FIG. 6D shows quantitative estimation of HTR of CS1− to CR+ by Sanger sequencing. Correction percentages are calculated for 3-5 individuals (2 reads per individual), using the double peaks marked by an asterisk in panel c (inset on the right) for indicated genotypes. Cas9 produces a significant correction (31%), while D10A yields to an even higher correction (˜52%). For the CS2− allele, correction values are higher for both Cas9 (˜53%) and D10A (˜59%).

FIG. 7A shows Cas9, D10A and H840A nucleases expressed under the control of vasa promoter from three equivalent insertions (in X-chromosome locus yellow) used to induce allelic repair in y^{ccw} w^{ATG- CR+}/y^{vasaCas9} w^{ATG+ CS1-}, y^{ccw} w^{ATG- CR+}/y^{vasaD10A} w^{ATG+ CS-}, and y^{ccw} w^{ATG- CR+}/y^{vasaH840A} w^{ATG+ CS1-} females. Control animals were y^{ccw} w^{ATG- CR+}/y⁺ w^{ATG+ CS1}, Left panels show typical repair phenotypes and diagrams on the right represent cutting or nicking by each different nuclease.

FIG. 7B shows allelic repair was quantified by image analysis using ImageJ. Nickases are more efficient than Cas9 in eliciting HTR, and H840A is more efficient than D10A for repairing the CS1− allele.

FIG. 7C shows quantitative analysis of HTR of the CS1− allele by deep-sequencing. Correction percentages after adjustments (see methods section) from five independent reads were plotted for each genotype (no nuclease control CR+/CS1−, +Cas9, +D10A, +H840A). Results confirm the trend calculated using pigment and Sanger sequencing analysis: repair by D10A is significantly higher (41%) than by Cas9 (27%). Interestingly, H840-elicited repair (51%) is significantly more efficient than by D10A.

FIG. 7D shows a pie-chart representation of Deep-sequencing analysis. Color coding—pink: donor CR+ alleles, white: intact CS1− alleles, dark purple: NHEJ mutations (centered at cut-site), grey: PCR-induced recombination #1 and some asymmetrical HTR and NHEJ events (only for Cas9, D10A and H840 samples, see figure S7), light blue sectors: PCR-induced recombination #2, light purple sectors: PCR-induced substitutions. This representation allows global visualization of different categories of events following Cas9, D10A and H840-dependent cleavage in w^{ATG-CR+}/y⁺ w^{ATG+ CS1-} individuals. Nickases are more effective at producing HTR than Cas9, with H840 inducing the highest levels of conversion. Cas9 induces high levels of NHEJ events, while D10A and H840 only elicit low levels of NHEJ and leave ˜18-23% of intact CS1− alleles.

FIG. 8A shows allelic repair involving two cut-sensitive (CS) white alleles: ATG+ CS− and del CS+. Cleavage of the CS− allele and repair using the CS+ sequences from the w^{del} allele (HDR-a) leads to a functional allele ATG+ CS+. Cleavage of the CS+ allele and repair using the CS− allele as template (HDR-b) leads to an non-functional allele del CS−. In both cases, the repaired allele remains cut-sensitive and susceptible to additional nuclease attacks, which may ultimately lead to functional or non-functional NHEJ alleles (Cas9) or remain mostly intact (D10A).

FIG. 8B shows clonal white+ phenotypes reveal DNA repair events for y^{ccw} w^{del CS+}/w^{ATG+ CS1-} (left images) and y^{ccw} w^{del CS+}/w^{ATG+ CS2-} (right images) animals expressing Cas9 (top images) or D10A (bottom images). When both alleles are cut-sensitive (CS1−/CS+), Cas9-dependent cleavage elicits very little repair (eyes are mostly white but few show a small red clone, upper left image). In contrast, y^{ccw} w^{del CS+}/w^{ATG+ CS2-} animals expressing Cas9 show consistently higher levels of red clones, indicating that the NHEJ pathway is predominantly responsible for allelic repair after Cas9-dependent cleavage of both cut-sensitive alleles. for D10A-expressing animals, both y^{ccw} w^{del CS+}/w^{ATG+ CS1-} (left bottom image) and y^{ccw} w^{del CS+}/w^{ATG+ CS2-} (right bottom image) show similar repair phenotypes consisting of numerous small white⁺ clones, reflective of predominant HTR.

FIG. 8C shows allele specific sequencing reveals repair in y^{ccw} w^{del CS+}/w^{ATG+ CS1-} genotypes (top diagram). The forward primer located at the ATG initiation codon ensures specific amplification of ATG+ sequences, but not from the w^{del} allele. In control animals (no nuclease, top chromatogram), only CS1− sequences are amplified and read. Asterisks indicate peaks used for quantitative analysis in FIG. 8D. Following Cas9-induced DSB (second electropherogram), double peaks starting at the CS1− deletion breakpoint (green line) reveal effective HTR. Triple and quadruple peaks starting at the cut site reveal high levels of NHEJ. Following D10A-induced nicks (third electropherogram), more pronounced double peaks reveal high levels of HTR (blue line), while no NHEJ is detectable after the cut site. Figure discloses SEQ ID NOS 38-41, respectively, in order of appearance.

FIG. 8D shows quantitative analysis of HTR by allele-specific sequencing in y^{ccw} w^{del CS+}/w^{ATG+ CS1-} animals. Correction percentages are plotted for each genotype. Cas9-induced repair (˜12%) is significantly lower than D10A-induced repair (˜36%).

FIG. 9A shows a system designed to reveal pairing-independent HDR by white+ clonal phenotypes, where repair template (transgene carrying a mini-white cDNA) is provided from a separate chromosomal location, after D10A-induced nicking.

FIG. 9B shows an autosomal P(mini-white+) transgenic insertion causes a light orange eye phenotype in otherwise white− background (left bottom panel). In y^{ccw} w^{ATG+ CS1-}/Y; P(mini-white⁺)/vasaCas9 males (2^{nd} panel), efficient DNA cleavage at the P(mini-white+) insertion causes large white− clones derived from NHEJ-mediated mutagenesis. In y^{ccw} w^{ATG+ CS1-}//Y; D10A/+; P(mini-white⁺)/+ males (3^{rd} panel), numerous small white+ clones in orange background indicate that allelic repair is occurring consistently using sequence homology, independently of chromosome pairing. In y^{ccw} w^{del CS+}/Y; D10A/+; P(mini-white⁺)/+ males (4^{th} panel), such repair is not detectable, as the w^{del} cannot be restored to a functional state. This latter control demonstrates that the white⁺ clones in y^{ccw} w^{ATG+ CS1-}/Y; D10A/+; P(mini-white⁺)/+ animals (3^{rd} panel) do not result from any alteration of the P(mini-white⁺) sequences but repair of the CS1− allele from the autosomal the P(mini-white⁺) sequences.

FIG. 9C shows quantification of pairing-independent repair of the CS1− allele reveal ˜8% phenotypic rescue induced by D10A compared to no rescue in control. In these experiments, 2^{nd} chromosome vasaD10A and 3^{rd} chromosome vasaCas9 were used.

FIG. 10 shows an exemplary schematic of conventional DNA repair with a Cas9 system versus a homologous chromosome-driven gene correction system.

FIG. 11 shows two exemplary cases when homologous chromosome-driven gene correction can be applicable.

FIG. 12 shows exemplary allele specific guide RNA strategies for repair of mutant alleles using homologous chromosome-driven gene correction. Figure discloses SEQ ID NOS 42-47, respectively, in order of appearance.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

### Definitions

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human or animal subject to be treated.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent. As used herein, and unless otherwise specified, the term “subject” is defined herein to include animals such as mammals, including, but not limited to, primates (e.g., humans), cows, sheep, goats, horses, dogs, cats, rabbits, rats, mice, and the like. In specific embodiments, the subject is a human. The terms “subject” and “patient” are used interchangeably herein in reference, for example, to a mammalian subject, such as a human.

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety.

“Amplification” refers to any known procedure for obtaining multiple copies of a target nucleic acid or its complement, or fragments thereof. The multiple copies may be referred to as amplicons or amplification products. Amplification, in the context of fragments, refers to production of an amplified nucleic acid that contains less than the complete target nucleic acid or its complement, e.g., produced by using an amplification oligonucleotide that hybridizes to, and initiates polymerization from, an internal position of the target nucleic acid. Known amplification methods include, for example, replicase-mediated amplification, polymerase chain reaction (PCR), reverse transcription polymerase chain reaction (RT-PCR), ligase chain reaction (LCR), strand-displacement amplification (SDA), and transcription-mediated or transcription-associated amplification. Amplification is not limited to the strict duplication of the starting molecule. For example, the generation of multiple cDNA molecules from RNA in a sample using reverse transcription (RT)-PCR is a form of amplification. Furthermore, the generation of multiple RNA molecules from a single DNA molecule during the process of transcription is also a form of amplification. During amplification, the amplified products can be labeled using, for example, labeled primers or by incorporating labeled nucleotides.

“Amplicon” or “amplification product” refers to the nucleic acid molecule generated during an amplification procedure that is complementary or homologous to a target nucleic acid or a region thereof. Amplicons can be double stranded or single stranded and can include DNA, RNA or both. Methods for generating amplicons are known to those skilled in the alt

“Complementary” or “complement thereof” means that a contiguous nucleic acid base sequence is capable of hybridizing to another base sequence by standard base pairing (hydrogen bonding) between a series of complementary bases. Complementary sequences may be completely complementary (i.e. no mismatches in the nucleic acid duplex) at each position in an oligomer sequence relative to its target sequence by using standard base pairing (e.g., G:C, A:T or A:U pairing) or sequences may contain one or more positions that are not complementary by base pairing (e.g., there exists at least one mismatch or unmatched base in the nucleic acid duplex), but such sequences are sufficiently complementary because the entire oligomer sequence is capable of specifically hybridizing with its target sequence in appropriate hybridization conditions (i.e. partially complementary). Contiguous bases in an oligomer are typically at least 80%, preferably at least 90%, and more preferably completely complementary to the intended target sequence.

“Configured to” or “designed to” denotes an actual arrangement of a nucleic acid sequence configuration of a referenced oligonucleotide. For example, a primer that is configured to generate a specified amplicon from a target nucleic acid has a nucleic acid sequence that hybridizes to the target nucleic acid or a region thereof and can be used in an amplification reaction to generate the amplicon. Also as an example, an oligonucleotide that is configured to specifically hybridize to a target nucleic acid or a region thereof has a nucleic acid sequence that specifically hybridizes to the referenced sequence under stringent hybridization conditions.

“Polymerase chain reaction” (PCR) generally refers to a process that uses multiple cycles of nucleic acid denaturation, annealing of primer pairs to opposite strands (forward and reverse), and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. There are many permutations of PCR known to those of ordinary skill in the art.

“Primer” refers to an enzymatically extendable oligonucleotide, generally with a defined sequence that is designed to hybridize in an antiparallel manner with a complementary, primer-specific portion of a target nucleic acid. A primer can initiate the polymerization of nucleotides in a template-dependent manner to yield a nucleic acid that is complementary to the target nucleic acid when placed under suitable nucleic acid synthesis conditions (e.g., a primer annealed to a target can be extended in the presence of nucleotides and a DNA/RNA polymerase at a suitable temperature and pH). Suitable reaction conditions and reagents are known to those of ordinary skill in the art. A primer is typically single stranded for maximum efficiency in amplification, but may alternatively be double stranded. If double stranded, the primer is generally first treated to separate its strands before being used to prepare extension products. The primer generally is sufficiently long to prime the synthesis of extension products in the presence of the inducing agent (e.g. polymerase). Specific length and sequence will be dependent on the complexity of the required DNA or RNA targets, as well as on the conditions of primer use such as temperature and ionic strength. Preferably, the primer is about 5-100 nucleotides. Thus, a primer can be, e.g., 5, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95 or 100 nucleotides in length. A primer does not need to have 100% complementarity with its template for primer elongation to occur; primers with less than 100% complementarity can be sufficient for hybridization and polymerase elongation to occur. A primer can be labeled if desired. The label used on a primer can be any suitable label, and can be detected by, for example, spectroscopic, photochemical, biochemical, immunochemical, chemical, or other detection means. A labeled primer therefore refers to an oligomer that hybridizes specifically to a target sequence in a nucleic acid, or in an amplified nucleic acid, under conditions that promote hybridization to allow selective detection of the target sequence.

A primer nucleic acid can be labeled, if desired, by incorporating a label detectable by, e.g., spectroscopic, photochemical, biochemical, immunochemical, chemical, or other techniques. To illustrate, useful labels include radioisotopes, fluorescent dyes, electron-dense reagents, enzymes (as commonly used in ELISAs), biotin, or haptens and proteins for which antisera or monoclonal antibodies are available. Many of these and other labels are described further herein and/or are otherwise known in the art. One of skill in the art will recognize that, in certain embodiments, primer nucleic acids can also be used as probe nucleic acids.

“Region” refers to a portion of a nucleic acid wherein said portion is smaller than the entire nucleic acid.

“Region of interest” refers to a specific sequence of a target nucleic acid that includes all codon positions having at least one single nucleotide substitution mutation associated with a genotype and/or subtype that are to be amplified and detected, and all marker positions that are to be amplified and detected, if any.

“RNA-dependent DNA polymerase” or “reverse transcriptase” (“RT”) refers to an enzyme that synthesizes a complementary DNA copy from an RNA template. All known reverse transcriptases also have the ability to make a complementary DNA copy from a DNA template; thus, they are both RNA- and DNA-dependent DNA polymerases. RTs may also have an RNAse H activity. A primer is required to initiate synthesis with both RNA and DNA templates.

“DNA-dependent DNA polymerase” is an enzyme that synthesizes a complementary DNA copy from a DNA template. Examples are DNA polymerase I from E. coli, bacteriophage T7 DNA polymerase, or DNA polymerases from bacteriophages T4, Phi-29, M2, or T5. DNA-dependent DNA polymerases may be the naturally occurring enzymes isolated from bacteria or bacteriophages or expressed recombinantly, or may be modified or “evolved” forms which have been engineered to possess certain desirable characteristics, e.g., thermostability, or the ability to recognize or synthesize a DNA strand from various modified templates. All known DNA-dependent DNA polymerases require a complementary primer to initiate synthesis. It is known that under suitable conditions a DNA-dependent DNA polymerase may synthesize a complementary DNA copy from an RNA template. RNA-dependent DNA polymerases typically also have DNA-dependent DNA polymerase activity.

“DNA-dependent RNA polymerase” or “transcriptase” is an enzyme that synthesizes multiple RNA copies from a double-stranded or partially double-stranded DNA molecule having a promoter sequence that is usually double-stranded. The RNA molecules (“transcripts”) are synthesized in the 5′-to-3′ direction beginning at a specific position just downstream of the promoter. Examples of transcriptases are the DNA-dependent RNA polymerase from E. coli and bacteriophages T7, T3, and SP6.

A “sequence” of a nucleic acid refers to the order and identity of nucleotides in the nucleic acid. A sequence is typically read in the 5′ to 3′ direction. The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

A “label” refers to a moiety attached (covalently or non-covalently), or capable of being attached, to a molecule, which moiety provides or is capable of providing information about the molecule (e.g., descriptive, identifying, etc. information about the molecule) or another molecule with which the labeled molecule interacts (e.g., hybridizes, etc.). Exemplary labels include fluorescent labels (including, e.g., quenchers or absorbers), weakly fluorescent labels, non-fluorescent labels, colorimetric labels, chemiluminescent labels, bioluminescent labels, radioactive labels, mass-modifying groups, antibodies, antigens, biotin, haptens, enzymes (including, e.g., peroxidase, phosphatase, etc.), and the like.

A “linker” refers to a chemical moiety that covalently or non-covalently attaches a compound or substituent group to another moiety, e.g., a nucleic acid, an oligonucleotide probe, a primer nucleic acid, an amplicon, a solid support, or the like. For example, linkers are optionally used to attach oligonucleotide probes to a solid support (e.g., in a linear or other logic probe array). To further illustrate, a linker optionally attaches a label (e.g., a fluorescent dye, a radioisotope, etc.) to an oligonucleotide probe, a primer nucleic acid, or the like. Linkers are typically at least bifunctional chemical moieties and in certain embodiments, they comprise cleavable attachments, which can be cleaved by, e.g., heat, an enzyme, a chemical agent, electromagnetic radiation, etc. to release materials or compounds from, e.g., a solid support. A careful choice of linker allows cleavage to be performed under appropriate conditions compatible with the stability of the compound and assay method. Generally a linker has no specific biological activity other than to, e.g., join chemical species together or to preserve some minimum distance or other spatial relationship between such species. However, the constituents of a linker may be selected to influence some property of the linked chemical species such as three-dimensional conformation, net charge, hydrophobicity, etc. Exemplary linkers include, e.g., oligopeptides, oligonucleotides, oligopolyamides, oligoethyleneglycerols, oligoacrylamides, alkyl chains, or the like. Additional description of linker molecules is provided in, e.g., Hermanson, Bioconjugate Techniques, Elsevier Science (1996), Lyttle et al. (1996) Nucleic Acids Res. 24(14):2793, Shchepino et al. (2001) Nucleosides, Nucleotides, & Nucleic Acids 20:369, Doronina et al (2001) Nucleosides, Nucleotides, & Nucleic Acids 20:1007, Trawick et al. (2001) Bioconjugate Chem. 12:900, Olejnik et al. (1998) Methods in Enzymology 291:135, and Pljevaljcic et al. (2003) J. Am. Chem. Soc. 125(12):3486, all of which are incorporated by reference.

“Fragment” refers to a piece of contiguous nucleic acid that contains fewer nucleotides than the complete nucleic acid.

“Hybridization,” “annealing,” “selectively bind,” or “selective binding” refers to the base-pairing interaction of one nucleic acid with another nucleic acid (typically an antiparallel nucleic acid) that results in formation of a duplex or other higher-ordered structure (i.e. a hybridization complex). The primary interaction between the antiparallel nucleic acid molecules is typically base specific, e.g., A/T and G/C. It is not a requirement that two nucleic acids have 100% complementarity over their full length to achieve hybridization. Nucleic acids hybridize due to a variety of well characterized physio-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel (Ed.) Current Protocols in Molecular Biology, Volumes I, II, and III, 1997, which is incorporated by reference.

The term “attached” or “conjugated” refers to interactions and/or states in which material or compounds are connected or otherwise joined with one another. These interactions and/or states are typically produced by, e.g., covalent bonding, ionic bonding, chemisorption, physisorption, and combinations thereof.

“Nucleic acid” or “nucleic acid molecule” refers to a multimeric compound comprising two or more covalently bonded nucleosides or nucleoside analogs having nitrogenous heterocyclic bases, or base analogs, where the nucleosides are linked together by phosphodiester bonds or other linkages to form a polynucleotide. Nucleic acids include RNA, DNA, or chimeric DNA-RNA polymers or oligonucleotides, and analogs thereof. A nucleic acid backbone can be made up of a variety of linkages, including one or more of sugar-phosphodiester linkages, peptide-nucleic acid bonds, phosphorothioate linkages, methylphosphonate linkages, or combinations thereof. Sugar moieties of the nucleic acid can be ribose, deoxyribose, or similar compounds having known substitutions (e.g. 2′-methoxy substitutions and 2′-halide substitutions). Nitrogenous bases can be conventional bases (A, G, C, T, U) or analogs thereof (e.g., inosine, 5-methylisocytosine, isoguanine). A nucleic acid can comprise only conventional sugars, bases, and linkages as found in RNA and DNA, or can include conventional components and substitutions (e.g., conventional bases linked by a 2′-methoxy backbone, or a nucleic acid including a mixture of conventional bases and one or more base analogs). Nucleic acids can include “locked nucleic acids” (LNA), in which one or more nucleotide monomers have a bicyclic furanose unit locked in an RNA mimicking sugar conformation, which enhances hybridization affinity toward complementary sequences in single-stranded RNA (ssRNA), single-stranded DNA (ssDNA), or double-stranded DNA (dsDNA). Nucleic acids can include modified bases to alter the function or behavior of the nucleic acid (e.g., addition of a 3′-terminal dideoxynucleotide to block additional nucleotides from being added to the nucleic acid). Synthetic methods for making nucleic acids in vitro are well known in the art although nucleic acids can be purified from natural sources using routine techniques. Nucleic acids can be single-stranded or double-stranded.

A nucleic acid is typically single-stranded or double-stranded and will generally contain phosphodiester bonds, although in some cases, as outlined, herein, nucleic acid analogs are included that may have alternate backbones, including, for example and without limitation, phosphoramide (Beaucage et al. (1993) Tetrahedron 49(10):1925 and references therein; Letsinger (1970) J. Org. Chem. 35:3800; Sprinzl et al. (1977) Eur. J. Biochem. 81:579; Letsinger et al. (1986) Nucl. Acids Res. 14: 3487; Sawai et al. (1984) Chem. Lett. 805; Letsinger et al. (1988) J. Am. Chem. Soc. 110:4470; and Pauwels et al. (1986) Chemica Scripta 26: 1419, which are each incorporated by reference), phosphorothioate (Mag et al. (1991) Nucleic Acids Res. 19:1437; and U.S. Pat. No. 5,644,048, which are both incorporated by reference), phosphorodithioate (Briu et al. (1989) J. Am. Chem. Soc. 111:2321, which is incorporated by reference), O-methylphosphoroamidite linkages (see Eckstein, Oligonucleotides and Analogues: A Practical Approach, Oxford University Press (1992), which is incorporated by reference), and peptide nucleic acid backbones and linkages (see, Egholm (1992) J. Am. Chem. Soc. 114:1895; Meier et al. (1992) Chem. Int. Ed. Engl. 31:1008; Nielsen (1993) Nature 365:566; and Carlsson et al. (1996) Nature 380:207, which are each incorporated by reference). Other analog nucleic acids include those with positively charged backbones (Denpcy et al. (1995) Proc. Natl. Acad. Sci. USA 92:6097, which is incorporated by reference); non-ionic backbones (U.S. Pat. Nos. 5,386,023, 5,637,684, 5,602,240, 5,216,141 and 4,469,863; Angew (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghvi and P. Dan Cook; Mesmaeker et al. (1994) Bioorganic & Medicinal Chem: Lett. 4: 395; Jeffs et al. (1994) J. Biomolecular NMR 34:17; and Tetrahedron Lett. 37:743 (1996), which are each incorporated by reference) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghvi and P. Dan Cook, which references are each incorporated by reference. Nucleic acids containing one or more carbocyclic sugars are also included within the definition of nucleic acids (see Jenkins et al. (1995) Chem. Soc. Rev. pp 169-176, which is incorporated by reference). Several nucleic acid analogs are also described in, e.g., Rawls, C & E News Jun. 2, 1997 page 35, which is incorporated by reference. These modifications of the ribose-phosphate backbone may be done to facilitate the addition of additional moieties such as labels, or to alter the stability and half-life of such molecules in physiological environments.

In addition to these naturally occurring heterocyclic bases that are typically found in nucleic acids (e.g., adenine, guanine, thymine, cytosine, and uracil), nucleic acid analogs also include those having non-naturally occurring heterocyclic or modified bases, many of which are described, or otherwise referred to, herein. In particular, many non-naturally occurring bases are described further in, e.g., Seela et al. (1991) Helv. Chim. Acta 74:1790, Grein et al. (1994) Bioorg. Med. Chem. Lett. 4:971-976, and Seela et al. (1999) Helv. Chim. Acta 82:1640, which are each incorporated by reference. To further illustrate, certain bases used in nucleotides that act as melting temperature (TO modifiers are optionally included. For example, some of these include 7-deazapurines (e.g., 7-deazaguanine, 7-deazaadenine, etc.), pyrazolo[3,4-d]pyrimidines, propynyl-dN (e.g., propynyl-dU, propynyl-dC, etc.), and the like. See, e.g., U.S. Pat. No. 5,990,303, entitled “SYNTHESIS OF 7-DEAZA-2′-DEOXYGUANOSINE NUCLEOTIDES,” which issued Nov. 23, 1999 to Seela, which is incorporated by reference. Other representative heterocyclic bases include, e.g., hypoxanthine, inosine, xanthine; 8-aza derivatives of 2-aminopurine, 2,6-diaminopurine, 2-amino-6-chloropurine, hypoxanthine, inosine and xanthine; 7-deaza-8-aza derivatives of adenine, guanine, 2-aminopurine, 2,6-diaminopurine, 2-amino-6-chloropurine, hypoxanthine, inosine and xanthine; 6-azacytosine; 5-fluorocytosine; 5-chlorocytosine; 5-iodocytosine; 5-bromocytosine; 5-methylcytosine; 5-propynylcytosine; 5-bromovinyluracil; 5-fluorouracil; 5-chlorouracil; 5-iodouracil; 5-bromouracil; 5-trifluoromethyluracil; 5-methoxymethyluracil; 5-ethynyluracil; 5-propynyluracil, and the like.

An “oligonucleotide” or “oligomer” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth. Enzymol. 68:90-99; the phosphodiester method of Brown et al. (1979) Meth. Enzymol. 68:109-151; the diethylphosphoramidite method of Beaucage et al. (1981) Tetrahedron Lett. 22:1859-1862; the triester method of Matteucci et al. (1981) J. Am. Chem. Soc. 103:3185-3191; automated synthesis methods; or the solid support method of U.S. Pat. No. 4,458,066, or other methods known in the art. All of these references are incorporated by reference.

In some embodiments, the Cas protein or the variant thereof is a Cas9 protein or a variant thereof. Isolated Cas9-crRNA complex from the S. thermophilus CRISPR-Cas system as well as complex assembled in vitro from separate components demonstrate that it binds to both synthetic oligodeoxynucleotide and plasmid DNA bearing a nucleotide sequence complementary to the crRNA. It has been shown that Cas9 has two nuclease domains—RuvC- and HNH-active sites/nuclease domains, and these two nuclease domains are responsible for the cleavage of opposite DNA strands. In some embodiments, the Cas9 protein is derived from Cas9 protein of S. thermophilus CRISPR-Cas system. In some embodiments, the Cas9 protein is a multi-domain protein having about 1,409 amino acids residues.

It should be appreciated that any CRISPR-Cas systems capable of disrupting the double stranded nucleic acid and creating a loop structure can be used in the present methods. For example, the Cas proteins provided herein may include, but not limited to, the Cas proteins described in Haft et al., PLoS Comput Biol., 2005, 1(6): e60, and Zhang et al., Nucl. Acids Res., 2013, 10.1093/nar/gkt1262. Some of these CRISPR-Cas systems require that a specific sequence be present for these CRISPR-Cas systems to recognize and bind to the target sequence. For instance, Cas9 may require the presence of a 5′-NGG protospacer-adjacent motif (PAM). Thus, in some embodiments, a PAM sequence or a sequence complementary to a PAM sequence is engineered into the target nucleic acid for initiating the binding of the CRISPR-Cas systems to the target nucleic acid.

As used herein, the term “guide polynucleotide”, refers to a polynucleotide sequence that can form a complex with an endonuclease (e.g., Cas protein such as Cas9) and enables the endonuclease to recognize and optionally cleave a target site on a polynucleotide such as DNA. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond, or linkage modification such as, but not limited, to locked nucleic acid (LNA), peptide nucleic acid (PNA), bridged nucleic acid (BNA), 5-methyl dC, 2,6-Diaminopurine, 2′-Fluoro A, 2′-Fluoro U, 2′-O-Methyl RNA, Phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. In some embodiments, the guide polynucleotide does not solely comprise ribonucleic acids (RNAs). In other embodiments, the guide polynucleotide does solely comprise ribonucleic acids (RNAs). A guide polynucleotide that solely comprises ribonucleic acids is also referred to as a “guide RNA”.

The guide polynucleotide can also be a single molecule comprising a first nucleotide sequence domain (referred to as Variable Targeting domain or VT domain) that is complementary to a nucleotide sequence in a target DNA and a second nucleotide domain (referred to as endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a guide polynucleotide can comprise a RNA sequence, a DNA sequence, or a RNA-DNA-combination sequence. In some embodiments, the guide polynucleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The guide polynucleotide being comprised of sequences from the crNucleotide and tracrNucleotide may be referred to as “guide RNA” (when composed of a contiguous stretch of RNA nucleotides) or “guide DNA” (when composed of a contiguous stretch of DNA nucleotides) or “guide RNA-DNA” (when composed of a combination of RNA and DNA nucleotides).

In general, a guide polynucleotide is any polynucleotide sequence having sufficient complementarity with a target polynucleotide sequence to hybridize with the target sequence and direct sequence-specific binding of a CRISPR complex to the target sequence. In some embodiments, the degree of complementarity between a guide polynucleotide and its corresponding target sequence, when optimally aligned using a suitable alignment algorithm, is about or at least about 50%, 55%, 60%, 65%, 70%, 75%, 80%, 85%, 90%, 95%, 96%, 97%, 98%, 99%, 99.5%, 99.9%, or 100%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide polynucleotide is about or at least about 5, 10, 11, 12, 13, 14, 15, 16, 17, 18, 19, 20, 21, 22, 23, 24, 25, 26, 27, 28, 29, 30, 35, 40, 45, 50, 75, or more than 75 nucleotides in length. In some embodiments, a guide polynucleotide is up to about 75, 50, 45, 40, 35, 30, 25, 20, 15, 12, or fewer than 12 nucleotides in length. The ability of a guide polynucleotide to direct sequence-specific binding of a CRISPR complex to a target sequence may be assessed by any suitable assay.

A “native gene” of an allele means the gene that is generally associated with the indicated allele of an organism. The “native gene” can refer to the wild type version of that gene or a mutant version of the gene.

### Homologous Chromosome Template Repair Systems and Methods

In one aspect is a homologous chromosome template repair (HTR) system described herein. In some embodiments, the HTR system comprises a gene editing system. In some embodiments, the gene editing system is configured to cut one allele of a cell but not the corresponding homologous allele. In some embodiments, the gene editing system is configured to cut a mutant allele of a cell at or near a mutation in the mutant allele but not cut a corresponding homologous allele without the mutation. In some embodiments, cleavage of one allele allows the corresponding homologous allele to act as a template for homology directed repair of the mutant allele. In some embodiments, this results in repairing the mutation in the mutant allele. In some embodiments, the gene editing system is configured to allow the corresponding homologous allele to act as a template for homology directed repair of the mutant allele. In some embodiments, the gene editing system is configured to allow the corresponding homologous allele to act as a template for homology directed repair of the mutant allele, thereby repairing the mutation in the mutant allele.

In another aspect provided herein is a method of performing a homologous chromosome template repair (HTR) in a cell. In some embodiments, the method comprises contacting a mutant allele with a gene editing system provided herein. In some embodiments, the gene editing system is configured to cut the mutant allele at or near a mutation in the mutant allele. In some embodiments, the gene editing system is configured to not cut the corresponding homologous allele without the mutation. In some embodiments, the method comprises cutting the mutant allele at or near the mutation in the mutant allele. In some embodiments, the method comprises use of the homologous allele as a template for homology directed repair (HDR). In some embodiments, the method results in the repair of the mutation in the mutant allele (e.g., the mutant allele is repaired to no longer include the mutation and/or to contain the same sequence at the mutation site as the homologous allele).

Gene editing systems for HTR systems and corresponding methods comprise the necessary components in order to effectuate the desired alterations to the desired allele. In some embodiments, the gene editing system comprises exogenous functionalities which work with endogenous systems (e.g., host cell proteins implicated in homology directed repair) to effectuate the desired alteration to the desired allele. In some embodiments, the gene editing system comprises a guide RNA. In some embodiments, the gene editing system comprises an endonuclease enzyme. In some embodiments, the gene editing system comprises a guide RNA and an endonuclease enzyme. In some embodiments, the gene editing system consists essentially of a guide RNA and an endonuclease enzyme (e.g., contains no other exogenous components necessary to effectuate the desired alterations to the desired allele). In some embodiments, the gene editing system consists of a guide RNA and an endonuclease enzyme. In some embodiments, the gene editing system does not comprise an exogenous repair template.

In some embodiments, the gene editing system comprises a guide RNA. In some embodiments, the guide RNA is configured to recruit the endonuclease enzyme (e.g., CRISPR-Cas9 and derivatives thereof) in order to effectuate a cut in the desired allele. In some embodiments, the guide RNA is configured to specifically hybridize to a desired allele (e.g., a mutant allele). In some embodiments, the guide RNA comprises a sequence of at least about 10, 15, 16, 17, 18, 19, 20, 21, 22, or 23 nucleotides complementary to the mutant allele. In some embodiments, the guide RNA is complementary to a portion of the mutant allele but not to the corresponding portion of the homologous allele.

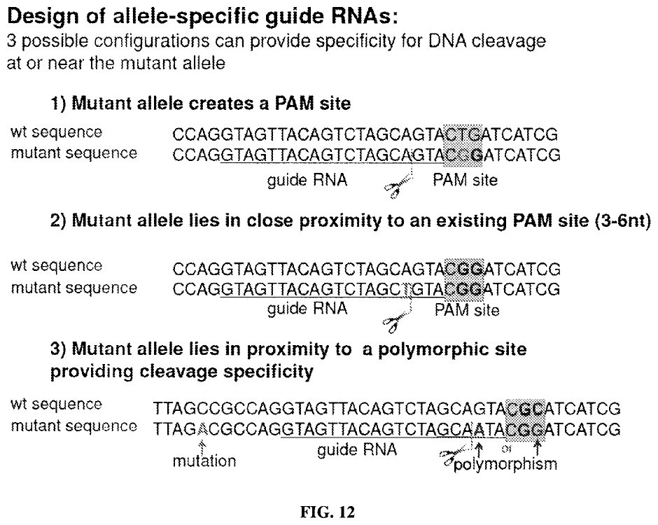

Exemplary design of strategies of guide RNAs configured to specifically cut a mutant allele are shown in FIG. 12. In some embodiments, the complementary portion of the guide RNA is complementary to a site of the mutant allele comprising the mutation. In some embodiments, the complementary portion of the guide RNA is complementary to a site of the mutant allele comprising a mutation which creates a protospacer adjacent motif (PAM) site in the mutant allele. In some embodiments, the complementary portion of the guide RNA is complementary to a site of the mutant allele near an endonuclease recognition site (e.g., a PAM site) which comprises the mutation. In some embodiments, the complementary portion of the guide RNA is complementary to a site of the mutant allele near a PAM site which comprises the mutation. In some embodiments, the complementary portion of the guide RNA is complementary to a site of the mutant allele which comprises a polymorphic site compared to the homologous allele. In some embodiments, the mutation is near the polymorphic site by not bound by the complementary portion of the guide RNA. In some embodiments, the mutation is within about 200, about 150, about 100, about 90, about 80, about 70, about 60, about 50, about 40, about 30, or about 20 nucleotides from the polymorphic site.

In some embodiments, the gene editing system comprises an endonuclease. In some embodiments, the endonuclease is configured to perform a cut in the mutant allele. In some embodiments, the endonuclease is configured to perform a cut in the mutant allele when in complex with the guide RNA. In some embodiments, the endonuclease is selected from a meganuclease, a Transcription Activator Like Effector Nucleases (TALEN), a Zinc-Finger Nucleases (ZFN), and a Clustered Regularly Interspaced Short Palindromic Repeats (CRISPR)-associated system (Cas), and derivatives thereof. In some embodiments, the endonuclease is a meganuclease or a derivative thereof. In some embodiments, the endonuclease is a TALEN or a derivative thereof. In some embodiments, the endonuclease is a ZFN or a derivative thereof. In some embodiments, the endonuclease is a Cas or a derivative thereof. In some embodiments, the endonuclease is a Cas9 or a derivative thereof. In some embodiments, the endonuclease is a Cas9 variant selected from D10A and H840A. In some embodiments, the endonuclease is D10A nickase. In some embodiments, the endonuclease is H840A nickase.

In some embodiments, the gene editing system is configured to perform a cut in the mutant allele. In some embodiments, the cut is a double-stranded cut (i.e., a double stranded break) or a single-stranded cut (i.e., a single stranded break). In some embodiments, the cut is a double-stranded cut. In some embodiments, the cut is a single-stranded cut. In some embodiments, the single-stranded cut is in the encoding strand of the mutant allele. In some embodiments, the singe-stranded cut is in the template strand of the mutant allele.

The HTR systems and methods provided herein can be used to repair a wide variety of mutations in the mutant allele. In some embodiments, the mutation comprises a nucleotide substitution at 1, 2, 3, 4, 5, 6, 7, 8, 9, 10, or more nucleotides. In some embodiments, the mutation is a single nucleotide substitution. In some embodiments, the mutation is an indel mutation. In some embodiments, the mutation is an insertion of 1, 2, 3, 4, 5, 6, 7, 8, 9, 10, or more nucleotides. In some embodiments, the mutation is a deletion of 1, 2, 3, 4, 5, 6, 7, 8, 9, 10, or more nucleotides. In some embodiments, the indel mutation is an in-frame or a frame-shit mutation. In some embodiments, the mutation is a single point mutation.

In some embodiments, the mutation in the mutant allele allows a gene editing system as provided herein (e.g., a gene editing system which comprises a guide RNA) to specifically target the mutant allele. Examples of these mutation can be found in FIG. 12. In some embodiments, the mutation is at a point at or near a potential cut site in the mutant allele that can be selectively targeted by a gene editing system provided herein (e.g., cut only in the mutant allele but not the homologous allele). In some embodiments, the mutation in the mutant allele creates an endonuclease recognition site in the mutant allele. In some embodiments, the endonuclease recognition site is a protospacer adjacent motif (PAM). In some embodiments, the mutation in the mutant allele is near an endonuclease recognition site present on both the mutant allele and the homologous allele. In some embodiments, the gene editing system is configured to cut the mutant allele at the mutation. In some embodiments, the mutation in the mutant allele is near a polymorphic site near an endonuclease recognition site. In some embodiments, the gene editing system is configured to cut the mutant allele at the polymorphic site. In some embodiments, the mutation is within about 200, about 150, about 100, about 90, about 80, about 70, about 60, about 50, about 40, about 30, or about 20 nucleotides from the cut site.

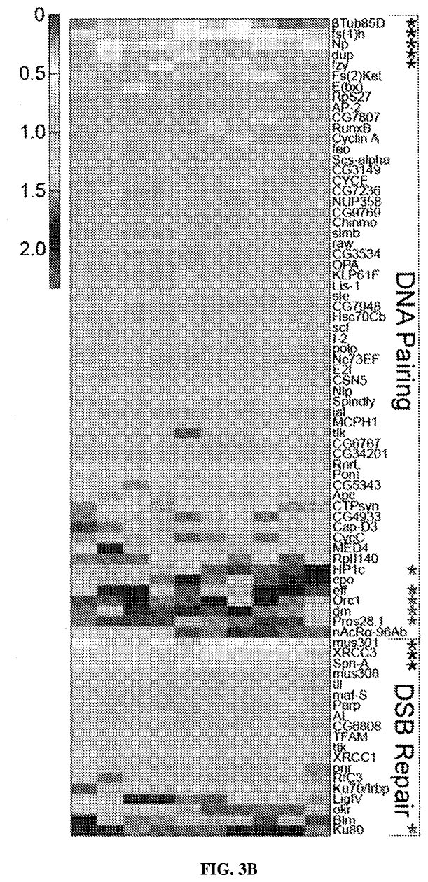

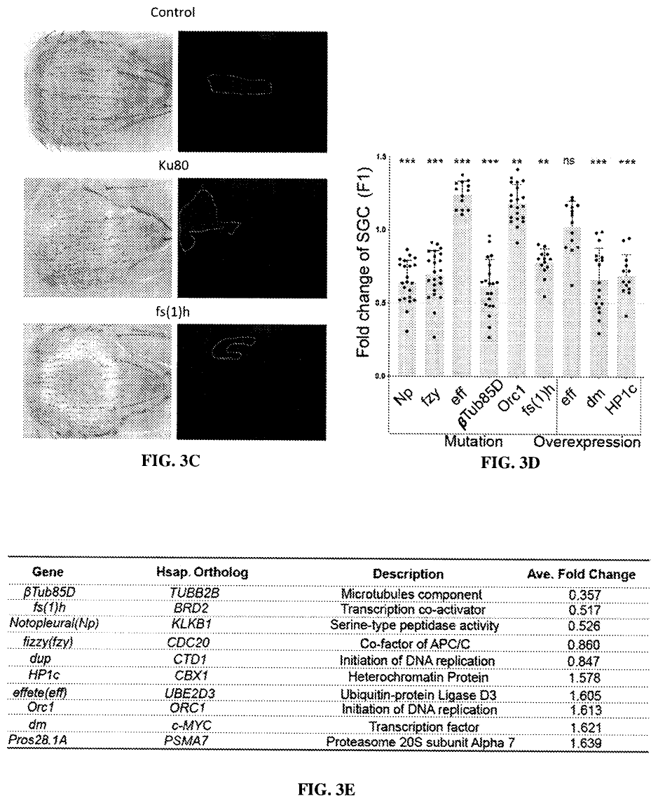

In some embodiments, the HTR systems and/or methods provided herein can comprise use of additional agents. In some embodiments, the HTR systems and/or methods comprise an agent which modulates the activity or expression of a protein implicated in a DNA repair pathway. Examples of such proteins include those listed in FIG. 3B and FIG. 3E. In some embodiments, the HTR systems and/or methods comprise an agent which modulates the activity or expression of a protein implicated in homology directed repair or non-homologous end joining, In some embodiments, such agents can increase the efficiency with which homologous chromosome template repair occurs in a cell or organism. In some embodiments, the agent enhances the activity or expression of a protein implicated homology directed repair of the cell or organism. In some embodiments, the agent inhibits the activity or expression of a protein implicated in an alternative DNA repair pathway. In some embodiments, the agent inhibits the activity or expression of a protein implicated in non-homologous end joining. In some embodiments, the agent is interfering RNA (RNAi). Other examples of agents include small-molecules, biologics, biosimilars, and the like. In some embodiments, the additional agent is administered concurrently with the HTR system. In some embodiments, the additional agent is administered separated from the HTR system. In some embodiments, the additional agent can be encoded on the same vector as the HTR system.

The HTR systems and methods provided herein are compatible with use in a wide variety of cells. In some embodiments, the cell is a eukaryotic cell. In some embodiments, cell is a plant cell, a fungal cell, or an animal cell. In some embodiments, the cell is a fungal cell (e.g., yeast). In some embodiments, the cell is an animal cell. In some embodiments, the cell is an insect cell, a mammalian cell, an avian cell, an amphibian cell, a reptilian cell, or a fish cell. In some embodiments, the cell is an insect cell. In some embodiments, the cell is a cell of Drosophila melanogaster. In some embodiments, the cell is a mammalian cell. In some embodiments, the cell is a human cell, a primate cell, a rodent cell, a canine cell, a bovine cell, an equine cell, or a porcine cell. In some embodiments, the cell is a human cell. In some embodiments, the cell is a somatic cell. In some embodiments, the cell is a human somatic cell.

In addition to cell cultures, the HTR systems and methods can also be used in whole organisms. In some embodiments, the organism is a multicellular organism. In some embodiments, the organism is an insect (e.g., Drosophila melanogaster, mosquito, etc.). In some embodiments, the organism is a plant. In some embodiments, the organism is a mammal. In some embodiments, the organism is a human. In some embodiments, the cell is a somatic cell of the organism. In some embodiments, the HTR system is functional in a somatic cell of a human.

In some embodiments, the HTR systems provided herein are encoded in a vector. In some embodiments, the gene editing systems provided herein are encoded in a vector. In some embodiments, the vector is a plasmid, a viral vector, a cosmid, or an artificial chromosome. In some embodiments, the vector is a plasmid, a viral vector, or a cosmid. In some embodiments, the vector is a plasmid or a viral vector. In some embodiments, the vector is a plasmid. In some embodiments, the vector is a viral vector. Examples of viral vectors include retrovirus, lentivirus, adenovirus, adeno-associated virus, herpes simplex virus, and the like.

In some embodiments, the HTR methods provided herein comprises delivery of HTR systems and/or gene editing systems provided herein, vectors encoding HTR systems and/or gene editing systems provided herein, or pharmaceutical compositions thereof to cells. In some embodiments, the method comprises introducing the gene editing system and/or the HTR system into a target cell. Delivery into the target cell can be accomplished by any appropriate method, including viral delivery (e.g., for viral vectors), delivery in lipid vesicles (e.g., exosomes, lipid nanoparticles, etc.), and the like. Methods of introducing a vector into a host cell are known in the art, and any known method is often used to introduce a vector into a cell. Suitable methods of delivery of HTR systems, gene editing systems, and/or vectors encoding the same include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, direct micro injection, nanoparticle-mediated nucleic acid delivery (see, e.g., Panyam et., al Adv Drug Deliv Rev. 2012 Sep. 13. pii: S0169-409X(12)00283-9. doi: 10.1016/j . addr.2012.09.023), and the like.

Also provided herein are pharmaceutical compositions of the HTR systems, gene editing systems, and vectors encoding the same. In some embodiments described herein is a pharmaceutical composition comprising an HTR system as provided herein and a pharmaceutically acceptable carrier. In some embodiments described herein is a pharmaceutical composition comprising a vector encoding an HTR system as provided herein and a pharmaceutically acceptable carrier. In some embodiments described herein is a pharmaceutical composition comprising a gene editing system as provided herein and a pharmaceutically acceptable carrier. In some embodiments described herein is a pharmaceutical composition comprising a vector encoding a gene editing system as provided herein and a pharmaceutically acceptable carrier.

The HTR systems and methods provided herein are useful for a variety of purposes. In one aspect is a method of repairing a mutation in a cell comprising introducing an HTR system provided herein to the cell. In another aspect is a method of repairing a mutation in a cell comprising introducing a vector encoding an HTR system provided herein into the cell. In some embodiments, the HTR system or the vector encoding the HTR system is introduced into the nucleus of the cell. In some embodiments, the HTR system or the vector encoding the HTR system contacts the target mutant allele of the cell.

Also provided herein is a method of performing gene therapy in a subject with an HTR system provided herein or a vector encoding an HTR system provided herein. In some embodiments, the subject is a whole organism as provided herein. In some embodiments, the gene therapy is effective to repair target mutant alleles in one or more cells of the subject. In some embodiments, the gene therapy is effective to repair target mutant alleles in a designated target tissue of the subject. In some embodiments, the invention can be used in methods for gene therapy to correct dominant mutations in heterozygous patients, for example but not limited to: Autosomal Dominant Polycystic Kidney Disease (ADPKD), Familial Hypercholesterolemia, Hemorrhagic Telangiectasia, Hereditary Spherocytosis, Marfan's Syndrome, Neurofibromatosis, congenital (papular) atrichia, and Cadasil syndrome. In some embodiments, the invention can be used in methods for gene therapy to correct transheterozygous combinations of recessive mutations (i.e., patient carries two different non-functional mutations in the same gene), for example but not limited to: Cystic fibrosis, haemochromatosis, and retinitis pigmentosa. The invention can be used in methods to alter histocompatibility genotypes to make patients competent recipients for a broader range of transplantations or blood transfusions.

Non-limiting examples of mutant alleles which can be repaired according to the methods and systems provided herein are shown in FIG. 11. In a first exemplary embodiment, a subject or target cell contains a dominant mutations in heterozygous alleles. In some embodiments, the subject or target cell contains one functional allele and one non-functional or reduced function second mutant allele homologous to the functional allele. The HTR systems provided herein are configured to allele specifically cut the mutant allele and direct homology directed repair using the functional allele as the template in order to repair the second mutant allele and restore function.

In a second exemplary embodiment, both alleles of a gene contain mutations at different locations of each allele both of which render the gene non-functional or with reduced functionality (e.g., allele 1 has a mutation at location A and allele 2 has a mutation at location B). In such a case, each allele is still capable of repairing the mutation present on the other allele to restore function. In such a case, targeting either mutation on one allele and facilitating homology directed repair with the other allele as the template is effective to return the targeted allele to a functional state. In some embodiments, the second allele is then targeted for a second round of repair (optionally in a same step) in order to produce two functioning alleles. An example of a relevant gene capable of being targeted with this strategy is cystic fibrosis transmembrane conductance regulator (CFTR).

### CopyCatcher Systems and Methods

Further provided herein are CopyCatcher systems for the measurement of DNA repair events, including homologous chromosome directed repair (HTR), as well as vectors encoding the systems, methods of using the systems, vectors encoding the systems, and cells and organisms which contain the systems.

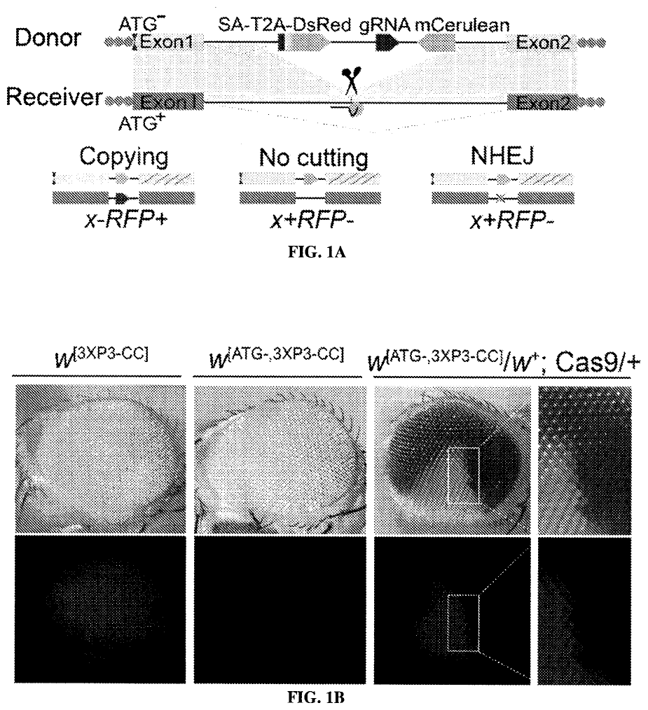

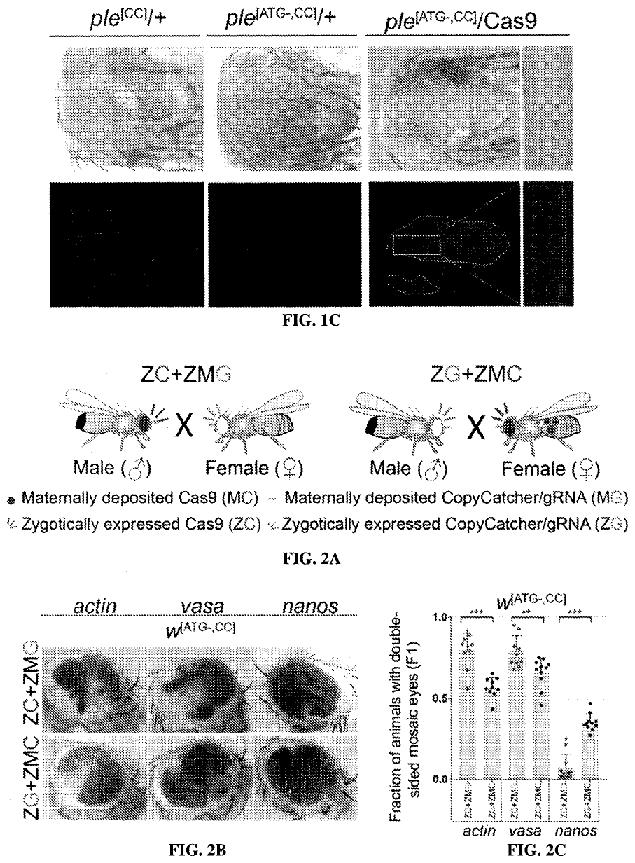

In one aspect, provided herein, is an engineered cell comprising a CopyCatcher system. In some embodiments, the engineered cell is useful for investigating homologous chromosome directed repair. In some embodiments, the engineered cell comprises a first allele which does not express an encoded gene (e.g., a native gene of the allele or a reporter gene placed within the allele). In some embodiments, the engineered cell comprises a second allele homologous to the first allele which contains a mutation relative to the first allele. In some embodiments, the engineered cell comprises a guide RNA which is configured to bind or recruit an endonuclease enzyme. In some embodiments, the nuclease enzyme is configured to facilitate or make a cut in the second allele at or near the mutation. In some embodiments, the nuclease enzyme is configured to make this cut when complexed with or bound to the guide RNA, such as when the guide RNA is also hybridized to the second allele. In some embodiments, the nuclease enzyme is also configured to not cut the first allele (e.g., the nuclease/guide RNA combination does not cut the first allele). In some embodiments, the system if configured such that homology directed repair (HDR) of the second allele (e.g., at or near the cut site) with the first allele as a template results in the transfer of the encoded gene of the first allele to then be encoded on the second allele (see, e.g., FIG. 1A). In some embodiments, the HDR results in the reporter gene being incorporated into the second allele. In some embodiments, the second allele does not encode the encoded gene of the first allele prior to homology directed repair using the first allele as a template.

In another aspect provided herein is a method for investigating DNA repair events (e.g., homologous chromosome template repair) in an organism using CopyCatcher elements. In some embodiments, the method comprises introducing into a first allele of an organism a first polynucleotide sequence which encodes a gene which will result in an observable change in the organism (e.g., a change in phenotype or a detectable signal) when the polynucleotide sequence is incorporated into a second allele of the cell or organism, wherein the second allele is homologous to the first allele. In some embodiments, the first polynucleotide sequence comprises a reporter gene. In some embodiments, the first polynucleotide sequence comprises at least a portion of a native gene or a mutation of the native gene which results in a change in phenotype in the organism when the portion of the native gene or the mutation of the native gene is incorporated into a second allele homologous to the first allele by homology directed repair. In some embodiments, the method comprises integrating a second polynucleotide sequence encoding a guide RNA configured to recruit or bind an endonuclease enzyme and facilitate a cut in the second allele at or near a mutation but not cut the first allele. In some embodiments, the method further comprises introducing the endonuclease or a vector encoding the endonuclease into the organism or a descendant of the organism. In some embodiments, the method comprises performing homology directed repair of the second allele with the first allele as a template. In some embodiments, the method results in the reporter gene or the portion of the native gene or the mutation of the native gene being incorporated into the second allele. In some embodiments, the second allele does not encode the encoded gene of the first allele prior to homology directed repair using the first allele as a template.

In some embodiments, the first allele of an organism comprising a CopyCatcher element as described herein does not express the encoded gene used for identification of successful homologous chromosome template repair (HTR) (e.g., the reporter gene, the native gene, or the variant/mutation of the native gene). In some embodiments, the first allele is modified so that the relevant encoded gene is not expressed. In some embodiments, the first allele is mutated so that the relevant encoded gene is not expressed. In some embodiments, the first allele is mutated in a parent organism of the one being examined. In some embodiments, the first allele is modified to not express the encoded gene by a mutation of or a deletion of a start codon. In some embodiments, the first allele is modified to not express the encoded bene by a deletion of a start codon. In some embodiments, the first allele is modified to not express the encoded gene through a frame-shift mutation.

In some embodiments, the encoded gene of the first allele comprises a reporter gene. The reporter gene is selected such that expression of the reporter gene can be easily and readily measured in the organism. In some embodiments, the reporter gene encodes a fluorescent protein, a bioluminescent protein, or a chromoprotein. Examples of such reporter genes and reporter proteins include green fluorescent protein and derivatives thereof, red fluorescent protein and derivatives thereof, luciferase and derivatives thereof, and the like.

In some embodiments, the reporter gene is configured in the first allele such that it is faithfully expressed in a functional state despite being incorporated into an existing native gene of the allele. In some embodiments, the reporter gene is preceded by a self-cleavage peptide. In some embodiments, the self-cleavage peptide is a 2A self-cleaving peptide (e.g., T2A, P2A, E2A, or F2A). In some embodiments, the self-cleavage peptide is a T2A peptide.

In some embodiments, the reporter gene is comprised in an intron of a native gene of the first allele. In some embodiments, additional genetic elements may be needed in order to properly express the reporter gene when it is comprised in the intron of the native gene of the allele. In some embodiments, the reporter gene is preceded on the first allele by a splice acceptor.

In some embodiments, the encoded gene of the first allele comprises a native gene of the first allele. In some embodiments, the second allele comprises a mutation in the native gene which results in an altered phenotype in the organism. In some embodiments, the second allele comprises a mutation in the native gene which results in an altered phenotype in the organism relative to an organism which expresses the native gene without the mutation. In some embodiments, the first allele encodes a wild type or healthy version of the native gene and the second allele encodes a mutant version of the native gene. In some embodiments, the first allele encodes a mutant version of the native gene and the second allele encodes a wild type or healthy version of the native gene. In some embodiments, the versions of the native gene on the first allele and the second allele result in different phenotypes when expressed by the organism. In some embodiments, the HDR with the first allele as the template results in an altered phenotype in the organism relative to an organism which did not undergo homology directed repair with the first allele as the template.

In some embodiments, the guide RNA which is configured to cut the second allele is encoded by the organism. In some embodiments, the guide RNA is encoded in the first allele. In some embodiments, the guide RNA is encoded in an intron of the first allele. In some embodiments, the guide RNA is encoded in an intron of a native gene of the first allele.

In some embodiments, the guide RNA is configured to recruit the endonuclease enzyme (e.g., CRISPR-Cas9 and derivatives thereof) in order to effectuate a cut in the second allele (i.e., the allele which does not contain the encoded gene to be transferred). In some embodiments, the guide RNA is configured to specifically hybridized to the second allele. In some embodiments, the guide RNA comprises a sequence of at least about 10, 15, 16, 17, 18, 19, 20, 21, 22, or 23 nucleotides complementary to the second allele. In some embodiments, the guide RNA is complementary to a portion of the second allele but not to the corresponding portion of the homologous allele.

In some embodiments, the second allele comprises a mutation relative to the first allele (i.e., the allele which contains but does not express the encoded gene which results in the detectable change when transferred to the second allele). In reference to CopyCatcher systems and methods provided herein, the mutation relative to the first allele and the second allele refers to any difference in nucleotide sequences between the alleles. In some embodiments, the mutation is that the second allele lacks the encoded gene of the first allele (e.g., the second allele encodes a functional version of the native gene and the first allele encodes a reporter gene). In some embodiments, the guide RNA which is used to cut the second allele and lead to homology directed repair of the second allele with the first allele as a template is the same guide RNA which was used in a previous step (e.g., in a germline descendant of the organism being investigated) which was used to insert the reporter gene or other portion of a gene which results in the change in phenotype in the organism when the portion of the gene is incorporated into the second allele. In some embodiments, the encoded gene of the first allele is a modification to the native gene of the allele such that the gene is rendered non-functional (e.g., a frame-shift mutation or functional nucleotide substitution). Thus, in some embodiments, the encoded gene of the first allele is a non-functional version of the native gene and the second allele encodes the native gene of the allele. Therefore, in some embodiments, the guide RNA is configured to introduce a cut in the native gene itself. In other embodiments, the encoded gene of the first allele is a functional version of the native gene and the mutation in the second allele is one which renders the encoded gene non-functional or with diminished functionality. In such cases, homology directed repair of the second allele with the first allele as a template results in the phenotype of the functional gene being observed in the organism.

In some embodiments, the mutation in the second allele relative to the first allele comprises a nucleotide substitution at 1, 2, 3, 4, 5, 6, 7, 8, 9, 10, or more nucleotides. In some embodiments, the mutation is a single nucleotide substitution. In some embodiments, the mutation is an indel mutation. In some embodiments, the mutation is an insertion of 1, 2, 3, 4, 5, 6, 7, 8, 9, 10, or more nucleotides. In some embodiments, the mutation is a deletion of 1, 2, 3, 4, 5, 6, 7, 8, 9, 10, or more nucleotides. In some embodiments, the indel mutation is an in-frame or a frame-shit mutation. In some embodiments, the mutation is a single point mutation. In some embodiments, the mutation in the second allele relative to the first allele comprises a deletion of the reporter gene encoded by the first allele. In such cases, the “deletion” refers to a sequence of nucleotides present in the first allele but not the second allele rather than the removal of nucleotides from the second allele.

Alternative design of strategies of guide RNAs configured to specifically cut the second allele are shown in FIG. 12. In some embodiments, the complementary portion of the guide RNA is complementary to a site of the second allele comprising the mutation. In some embodiments, the complementary portion of the guide RNA is complementary to a site of the second allele comprising a mutation which creates a protospacer adjacent motif (PAM) site in the mutant allele. In some embodiments, the complementary portion of the guide RNA is complementary to a site of the second allele near an endonuclease recognition site (e.g., a PAM site) which comprises the mutation. In some embodiments, the complementary portion of the guide RNA is complementary to a site of the second allele near a PAM site which comprises the mutation. In some embodiments, the complementary portion of the guide RNA is complementary to a site of the second allele which comprises a polymorphic site compared to the first allele. In some embodiments, the mutation is near the polymorphic site by not bound by the complementary portion of the guide RNA. In some embodiments, the mutation is within about 200, about 150, about 100, about 90, about 80, about 70, about 60, about 50, about 40, about 30, or about 20 nucleotides from the polymorphic site.

In some embodiments, the CopyCatcher systems (e.g., engineered cells or organisms as provided herein) and methods provided herein comprise or use an endonuclease enzyme. The endonuclease enzyme can be delivered into a target cell or organism in a variety of manners. In some embodiments, a gene encoding the endonuclease enzyme is introduced into the genome of the organism or an ancestor of the organism being immediately examined. In some embodiments, the endonuclease enzyme is delivered to the organism (e.g., a target cell of the organism) as an expressed protein using any suitable methods (e.g., electroporation, delivery with lipid vesicles, etc.). In some embodiments, the endonuclease enzyme is delivered through a vector encoding the endonuclease enzyme (e.g., a plasmid or viral vector configured to be delivered to the appropriate cell of an organism being investigated). In such embodiments, the vector then expresses the endonuclease enzyme in the desired cell.

In some embodiments, the endonuclease enzyme is selected from a meganuclease, a Transcription Activator Like Effector Nucleases (TALEN), a Zinc-Finger Nucleases (ZFN), and a Clustered Regularly Interspaced Short Palindromic Repeats (CRISPR)-associated system (Cas), and derivatives thereof. In some embodiments, the endonuclease is a meganuclease or a derivative thereof. In some embodiments, the endonuclease is a TALEN or a derivative thereof. In some embodiments, the endonuclease is a ZFN or a derivative thereof. In some embodiments, the endonuclease is a Cas or a derivative thereof. In some embodiments, the endonuclease is a Cas9 or a derivative thereof. In some embodiments, the endonuclease is a Cas9 variant selected from D10A and H840A. In some embodiments, the endonuclease is D10A nickase. In some embodiments, the endonuclease is H840A nickase.

In some embodiments, the endonuclease enzyme is configured to perform a cut in the mutant allele. In some embodiments, the cut is a double-stranded cut (i.e., a double stranded break) or a single-stranded cut (i.e., a single stranded break). In some embodiments, the cut is a double-stranded cut. In some embodiments, the cut is a single-stranded cut. In some embodiments, the single-stranded cut is in the encoding strand of the mutant allele. In some embodiments, the singe-stranded cut is in the template strand of the mutant allele.

In some embodiments, the CopyCatcher systems and/or methods provided herein can be used to determine the effect other genes and or agents have on DNA repair processes. In some embodiments, the systems and/or methods comprise an agent or administering an agent which modulates the activity or expression of a protein implicated in a DNA repair pathway. Examples of such proteins include those listed in FIG. 3B and FIG. 3E. In some embodiments, the systems and/or methods comprise an agent which modulates the activity or expression of a protein implicated in homology directed repair or non-homologous end joining, In some embodiments, such agents can increase the efficiency with which homologous chromosome template repair occurs in a cell or organism. In some embodiments, the agent enhances the activity or expression of a protein implicated homology directed repair of the cell or organism. In some embodiments, the agent inhibits the activity or expression of a protein implicated in an alternative DNA repair pathway. In some embodiments, the agent inhibits the activity or expression of a protein implicated in non-homologous end joining. In some embodiments, the agent is interfering RNA (RNAi). Other examples of agents include small-molecules, biologics, biosimilars, and the like. In some embodiments, the additional agent is administered concurrently with the endonuclease or a vector encoding the endonuclease to the organism. In some embodiments, the additional agent is administered separately from the endonuclease system (e.g., in a pre-treatment step). In some embodiments, the additional agent can be encoded on the same vector as the endonuclease enzyme. Such agents can be used to determine the effect of various genes and/or substances on DNA repair pathways by comparison to a reference organism which did not encounter the agent. In some embodiments, a method provided herein comprises a step of comparing expression of the reporter gene or the change in phenotype in the organism or the descendant of the organism and comparing the expression or change in phenotype to a reference organism which did not receive the agent. Such examination can be in one or more cells of an organism (for a multicellular organism) or made by examining a percentage of cells which contain the change in phenotype or expression (e.g., in a cell culture experiment).

The CopyCatcher systems and methods provided herein are compatible with use in a wide variety of cells. In some embodiments, the cell is a eukaryotic cell. In some embodiments, cell is a plant cell, a fungal cell, or an animal cell. In some embodiments, the cell is a fungal cell (e.g., yeast). In some embodiments, the cell is an animal cell. In some embodiments, the cell is an insect cell, a mammalian cell, an avian cell, an amphibian cell, a reptilian cell, or a fish cell. In some embodiments, the cell is an insect cell. In some embodiments, the cell is a cell of Drosophila melanogaster. In some embodiments, the cell is a mammalian cell. In some embodiments, the cell is a human cell, a primate cell, a rodent cell, a canine cell, a bovine cell, an equine cell, or a porcine cell. In some embodiments, the cell is a human cell. In some embodiments, the cell is a somatic cell.

In addition to cell cultures, the CopyCatcher systems and methods can also be used in whole organisms. In some embodiments, the organism is a multicellular organism. In some embodiments, the organism is an insect (e.g., Drosophila melanogaster, mosquito, etc.). In some embodiments, the organism is a plant. In some embodiments, the organism is a mammal. In some embodiments, the cell is a somatic cell of the organism.

### EXAMPLES

### Example 1—Use of CopyCatchers for the Investigation of Homologous Chromosome-Directed Repair (HTR) in Somatic Cells

### Results

### Architecture of Active Genetic CopyCatcher Systems

We sought to resolve the nature of somatic mutations generated through the action of gene drives using novel active genetic elements referred to as CopyCatchers designed to detect and quantify potential somatic copying events. CopyCatchers include a guide RNA (gRNA) for copying themselves at their site of genomic insertion into the introns of target genes and also harbor a genetic cassette that marks individual and descendant clones of cells in which these elements have been copied to the homologous chromosome. Such clones are delineated both by expression of a fluorescent marker (DsRed) and by creation of visible adult phenotypes (FIG. 1A). CopyCatchers also carry a T2A-DsRed transgene preceded by a strong splice acceptor (SA) that hijacks the original splicing of the target gene, thus generating in-frame fusion products between endogenous gene coding sequences and the DsRed reporter, which are thereby expressed under the control of native cis-regulatory sequences. The rationale for the DsRed reporter gene being preceded by a T2A self-cleavage peptide is to avoid potential signal quenching that might arise in direct protein fusions with endogenously encoded peptides where protein folding of the novel juxtaposed domains would be unpredictable. In addition, CopyCatchers include a separate conventional dominant fluorescent eye marker (3XP3-mCerulean) for tracking the element in genetic crosses (FIG. 1A). In-frame fusion of the T2A-DsRed reporter with the endogenous gene also results in truncation of endogenous gene transcripts, thus generating recessive loss-of-function alleles in the target gene.

Transgenic flies carrying CopyCatchers were associated in-cis with translation disruptive mutations upstream of the CopyCatcher insertion site to render DsRed expression conditional upon copying of the element to a wild-type chromosome. These translation abrogating ATG⁻ mutations were placed sufficiently far from the CopyCatcher insertion site so as to be outside of HDR-mediated copying range (≥1 kb from the gRNA cut site, which is well beyond the 150-200 bp range typically associated with localized directional gene-conversion events, which are initiated by 5′→3′ resection accompanying HDR repair, followed by synthesis dependent strand annealing (SDSA) and potential D-loop migration and resolution (FIG. 1A)³⁷⁻⁴⁶. DsRed fluorescence can be restored, however, if the elements copy themselves onto the wild-type homolog allele in a Cas9-dependent fashion thereby separating themselves from the linked ATG⁻ mutations. Such copying events would also generate homozygous mutant clones of descendant cells. Uncut alleles and any NHEJ indels generated in the process, however, should be phenotypically silent since CopyCatchers are inserted into non-essential intronic sites (FIG. 1A “No cutting” and “NHEJ”).

We inserted CopyCatchers into three different loci: white (w), Tyrosine 3-monooxygenase or pale (ple), and yellow (y). As predicted, each of these CopyCatchers created recessive mutant alleles that displayed readily identifiable pigmentation defects when homozygous (FIG. 1B and FIG. 1C). For ease of reference, we denote these three CopyCatcher transformant lines as w^{[CC]}, ple^{[CC]}, and y^{[CC]} respectively. The three CopyCatchers also expressed the DsRed protein in patterns conforming to the endogenous targeted gene (FIG. 1B and FIG. 1C: lower panels, first column). In addition, the homozygous w^{[CC]} and y^{[CC]} CopyCatchers exhibited strong loss-of-function pigmentation phenotypes (white eyes and yellow bodies respectively, FIG. 1B: top panel first column). Since the ple gene is essential for viability, the ple^{[CC]} CopyCatcher insertion was maintained as a balanced heterozygous stock (ple^{[CC]}/TM6). Homozygous patches of ple^{[CC]}/ple^{[CC]} mutant tissue could be generated in the presence of Cas9, and these clones displayed fully penetrant loss-of-pigmentation phenotypes as described further below (FIG. 1C: top panels, third and fourth columns).

Following the scheme outlined above, we next combined the DsRed⁺ CopyCatcher elements with 5′ translation disruptive ATG⁻ mutations and denoted these recombinant DsRed⁻ CopyCatcher alleles as w^{[ATG-,CC]} ple^{[ATG-,CC]} and y^{[ATG-,CC]} (FIG. 1B and FIG. 1C). Heterozygous ATG⁻ CopyCatchers were tested by placing them in-trans to a wild-type (+) allele and combining them with different of Cas9 sources expressed under the control of distinct promoters inserted at different chromosomal locations. Three Cas9-dependent outcomes are possible in somatic cells of such individuals: copying to the homolog chromosome (FIG. 1A “Copying”), no cutting (FIG. 1A “No cutting”), or generation of NHEJ-induced indels (FIG. 1A “NHEJ”). Among these three alternatives, only “copying”, mediated by cutting at the targeted site on the receiver chromosome followed by gene conversion with CopyCatcher sequences, would separate the elements from their linked 5′ ATG⁻ mutations permitting expression of the DsRed fusion protein in these cells and their mitotic descendants (FIG. 1A “Copying”). Also, as mentioned above, clonal adult tissues derived from these DsRed⁺ cells should display homozygous loss-of-function phenotypes (e.g., w^{[ATG-,CC]}=white eyes; ple^{[ATG-,CC]}=pale unpigmented bristles and thoracic epidermis; y^{[ATG-,CC]}=yellow bodies) (FIG. 1B and FIG. 1C: third and fourth columns,). Note again, that both small NHEJ-induced indels or non-cutting events should result in DsRed⁻ and wild-type pigmentation phenotypes (FIG. 1A “No cutting” and “NHEJ”).

### Concordance of CopyCatcher SGC Induced Fluorescent and Mutant Phenotypes

Initial tests of CopyCatchers revealed that Cas9-induced somatic gene conversion (SGC) events were readily observed and resulted in DsRed⁺ expressing cells coinciding with adult clones exhibiting mutant pigmentation phenotypes (FIG. 1B and FIG. 1C). We characterized the concordance of these two CopyCatcher phenotypes further. In the case of the homozygous y^{[CC]} element, DsRed expression closely followed that of the endogenous y gene in larval epidermal cells giving rise to ventral denticle hairs⁴⁷. During subsequent developmental stages (e.g., adults), however, specific fluorescence was difficult to detect since the endogenous y gene is only weakly expressed at these stages. As expected, combining the y^{[CC]} element with the upstream y^{[1]} allele (y^{[ATG-,CC]}/y^{[ATG-,CC]}) resulted in loss of the DsRed signal. When the y^{[ATG-,CC]} chromosome was placed in-trans to a wild-type X-chromosome in the presence of Cas9^{[ATG-,CC]}/+; Cas9/+), however, we observed individual DsRed⁺ cells and y⁻ denticles in corresponding positions, providing proof-of-principle for the CopyCatcher concept.

We also analyzed the w^{[CC]} and ple^{[CC]} elements in greater detail with particular interest in establishing robustly expressed DsRed reporters during adult stages that would enable facile and accurate quantification of SGC events. ple^{[CC]}/+ (or ple^{[CC]}/TM6) flies exhibited strong DsRed expression in epidermal nuclei throughout the adult thorax (FIG. 1C: first column, lower panel). The fluorescent signal in eyes of homozygous w^{[CC]} flies driven by the endogenous w promoter, however, was rather faint due to low levels of white gene expression during late phases of eye development. We therefore employed CRISPR editing to boost expression by inserting the artificial 3XP3 eye-specific promoter upstream of the translation initiator ATG codon of the w locus. When this w^{[3XP3]} allele was combined with the w^{[CC]} CopyCatcher (w^{[3XP3-CC]}), strong reproducible eye-specific expression of the DsRed reporter was indeed observed (FIG. 1B: first column, lower panel).

As expected, combining both the w^{[3XP3-CC]} and ple^{[CC]} elements with 5′ translation disrupting mutations eliminated their respective DsRed signals (FIG. 1B and FIG. 1C: second columns). When these dual mutant alleles were placed in-trans to wild-type chromosomes in the presence of Cas9, however, DsRed reporter expression was restored in precise one-to-one correspondence with loss-of-function mutant phenotypes in individual bristles indicative of accurate SGC (FIG. 1C: third and fourth columns). In the case of w^{[ATG-,3XP3-CC]}/+; Cas9/+ females (only females could be scored since w is on the X-chromosome), nearly all flies exhibited mosaic eyes with at least one eye having large white patches in a background of wild-type (red) pigmented cells. All such clonal sectors of white ommatidia also expressed DsRed (FIG. 1B: third column). Similarly, both male and female ple^{[ATG-,CC]}/Cas9 individuals displayed numerous thoracic patches of pale bristles faithfully coinciding with underlying DsRed⁺ epidermal nuclei, indicative of precise CopyCatcher-driven SGC events and concomitant homozygous ple loss-of-function in these clones (FIG. 1C: third and fourth columns). The reliable concordance of fluorescence and mutant phenotypes for both the w^{[ATG-,3XP3-CC]} and ple^{[ATG-,CC]} elements validates CopyCatchers as efficient and precise SGC tracking systems.

Quantifying SGC Events with the White and ple CopyCatcher Elements

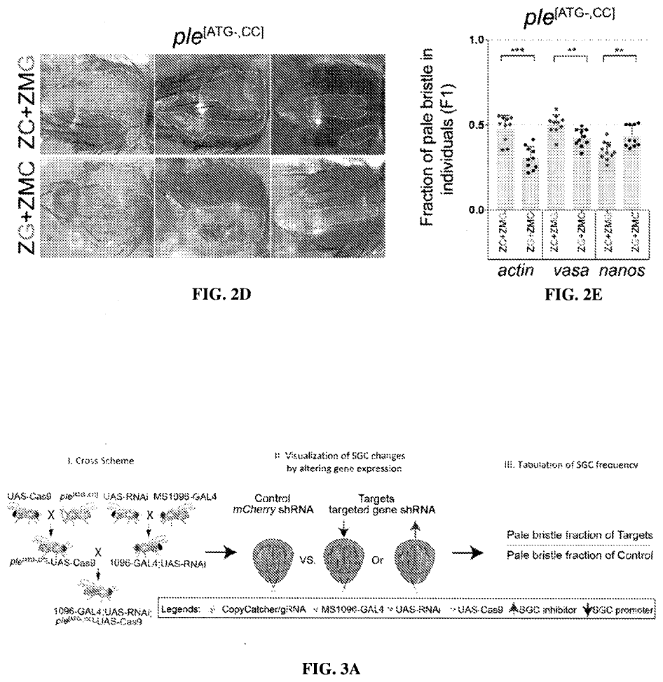

Drosophila offers flexible genetic tools for dissecting and optimizing genetic processes such as assessing how tissue specificity, timing, or levels of Cas9 expression might affect rates of CopyCatcher induced SGC events. We placed w^{[ATG-,CC]} and ple^{[ATG-,CC]} CopyCatcher double cis-mutant allelic combinations in-trans to wild-type alleles and evaluated SGC efficiency by semi-quantitative (w^{[ATG-,CC]}) and quantitative (ple^{[ATG-,CC]}) measures when using three different X-linked sources of Cas9: actin-Cas9 (ubiquitously expressed during the whole developmental stages)⁴⁸, vasa-Cas9 (expressed majorly in germline cells at all developmental stages, as well as embryonic somatic gonadal precursor cells)⁴⁹ and nanos-Cas9 (specifically transcribed in the nurse cells and protein was restrictedly distributed in the oocyte posterior and posterior of embryos)⁵⁰. We conducted crosses in which the Cas9 transgene was provided either paternally (denoted Zygotic Cas9 or ZC, FIG. 2A) or maternally (denoted as Zygotic plus Maternal Cas9 or ZMC, FIG. 2A) and determined how differing temporal and spatial patterns as well as levels of Cas9 might impact rates of SGC. In the case of the w^{[ATG-,CC]} element, we employed the original w^{[ATG-,CC]} line (without the inserted 5′ 3XP3 artificial promoter) to perform the clonal analysis since the low levels of DsRed driven by the endogenous w promoter do not interfere with scoring the Cas9-associated DsRed selection marker common to the three Cas9 lines tested (all of these Cas9 elements were inserted into the same target site in the y locus).

Virtually 100% of F₁ CopyCatcher females carrying Cas9 (y⁺w^{[ATG-,CC]}/y^{[Cas9]}w⁺ and y^{[Cas9]}/+; ple^{[ATG-,CC]}/+ genotypes) displayed extensive mosaic mutant phenotypes, for all sources of Cas9 and for both paternal and maternal crossing schemes (FIG. 2B and FIG. 2D). These results confirm that SGC is surprisingly frequent, occurring with complete penetrance, and that the w^{[CC]} and ple^{[CC]} CopyCatchers are highly efficient in detecting such copying events. We analyzed the extent of SGC events by scoring the fraction of flies having mosaic patches in both eyes of y⁺w^{[ATG-,CC]}/y^{[Cas9]}w⁺ females (a semiquantitative index) (FIG. 2C), and by tabulating the fraction of enumerated pale thoracic bristles for y^{[Cas9]}/+; ple^{[ATG-,CC]}/+ individuals (a quantitative index) (FIG. 2E).

In paternal Cas9 crosses (ZC♂+ZMG♀, FIG. 2A), both y⁺w^{[ATG-,CC]}/y^{[Cas9]}w⁺ and y^{[Cas9]}/+; ple^{[ATG-,CC]}/+ females exhibited SGC frequencies that increased according to inferred somatic Cas9 levels, with mosaic patches occurring most frequently in the following order of promoter-drive Cas9 expression: actin>vasa>nanos (FIGS. 2B-2E). This trend was also mirrored in F₁ progeny from maternal crossing schemes (ZG♂+ZMC♀, FIGS. 2B-2E). For paternal crosses in which Cas9 was driven by the actin and vasa promoters, ˜80% of y⁺w^{[ATG-,CC]}/y^{[Cas9]}w⁺ individuals displayed w⁻ clones in both eyes. Notably, however, maternal crossing schemes for supplying Cas9 resulted in significantly lower SGC frequencies in F₁ CopyCatcher progeny (e.g., 57% for actin-Cas9 and 66% for vasa-Cas9) than observed for paternal crossing schemes employing the same Cas9 sources (FIG. 2B and FIG. 2C). The reductions in SGC associated with maternal versus paternal pedigrees were particularly evident when crossing CopyCatcher males to actin-Cas9 females (the countervailing exception of w^{[ATG-,CC]} or ple^{[ATG-,CC]} males crossed to nanos-Cas9 females was analyzed in data not shown). A similar trend was evident for y^{[Cas9]}/+; ple^{[ATG-,CC]}/+ females, in which about half of all thoracic bristles displayed pale phenotypes for the actin-Cas9 and vasa-Cas9 source (SGC percentages were respectively: 48% and 50%) in paternal crosses versus 31% and 42% respectively for the corresponding maternal crosses (FIG. 2E). One explanation for these results is that SGC frequencies increase with overall Cas9 levels but decrease in response to maternal accumulation of Cas9/gRNA complexes in the egg, which can act at an early developmental stage to induce cleavage resistant NHEJ alleles precluding SGC during subsequent stages. Several other crossing schemes further support the hypothesis that higher levels of Cas9 delivered at later developmental stages optimize SGC.

The differing rates of SGC observed in paternal versus maternal CopyCatcher crossing schemes raised the possibility that the ability of the gRNAs to gain access to their chromosomal targets might differ between these two crossing schemes. We addressed this possibility in two ways. First, we tested CopyCatcher elements for efficiency of germline transmission, which provides a standardized measure for HDR-mediated DSB repair in meiotic lineages. In these experiments, F₁ trans heterozygous y⁺w^{[ATG-,CC]}/y^{[Cas9]}w⁺ or y^{[Cas9]}/+; ple^{[ATG-,CC]}/+ females were crossed to w¹¹⁸ males. Among F₂ progeny from both paternal and maternal crossing of w^{[ATG-,CC]} over 90% of individuals were positive for both CFP (the dominant CopyCatcher marker) and the white eye phenotype, representing a composite of both donor and receiver chromosomes carrying the CopyCatcher elements. Similarly, among F₂ progeny of ple^{[ATG-,CC]} crosses, at least 82% flies were CFP⁺ and 42% flies (=84% germline gene conversion) were RFP⁺ throughout the thorax, which selectively scored transmission of the HDR converted receiver chromosome. In contrast, the static RFP-marked Cas9 element, serving as an internal control, displayed standard Mendelian transmission (˜50% inheritance). These results indicate that both gRNAs employed in the w^{[CC]} and ple^{[CC]} CopyCatchers mediate efficient target cleavage and HDR-mediated copying in the germline.

As a complementary approach, we performed next-generation sequencing (NGS) on genomic DNA samples from two typical CopyCatcher crossing schemes using the vasa-Cas9 source. In this analysis, the fraction of uncut wild-type alleles was <5% of the total alleles recovered on the non-converted target chromosomes (the remainder were NHEJ indels, which differed in prevalence and abundance of specific alleles based on the crossing scheme). These NHEJ events altered sequences at varying distances from the gRNA cutting sites, but even the largest lesions did not extend into neighboring exons. Collectively, these findings suggest that the gRNAs carried by the w^{[CC]} and ple^{[CC]} CopyCatchers are highly efficient in cutting target chromosomes, and that the differing rates of SGC observed in various crossing scenarios can most likely be attributed to particular developmental patterns and levels of Cas9 expression. We hypothesize that these variations in Cas9 expression determine a balance between NHEJ (dominating during early embryonic stages of development) and somatic HDR-mediated repair.

A targeted screen identifies genes influencing CopyCatcher-induced SGC events Efforts to boost levels or activity of key HDR pathway components or to reduce the activities of competing NHEJ components typically produce modest increments in HDR/NHEJ ratios in mammalian cells, but still fall short of efficiencies required for many potential applications. It is unclear, however, whether other components involved DNA repair or chromosome pairing also contribute to such inter-chromosomal somatic correction. We speculated that factors altering rates of SGC might similarly impact HDR efficiencies in mammalian contexts.

As a first step in defining factors that influence SGC, we screened 109 of the Drosophila TRiP RNAi collection, to determine whether any of these genes when knocked down, might lead to differing rates of ple^{[ATG-,CC]}-mediated SGC events (FIG. 3A)^{51,52}. Targeted expression of the various RNAi lines was induced using the GAL4/UAS transactivation system (FIG. 3A)⁵³. We chose the ple^{[ATG-,CC]} CopyCatcher for these experiments since it was best suited for quantification of SGC events. We surveyed a set of 77 DNA pairing factors (DNA pairing) and 32 genes associated with DSB repair pathway factors (DSB repair, FIG. 3B). As positive controls we included Irbp (Drosophila Ku70 ortholog), Ku80 and DNA-ligIV, which we predicted should increase SGC and Spn-A (Drosophila Rad51 ortholog), which ought to decrease SGC in response to RNAi knock-down.

We screened candidate SGC modifiers by generating test females carrying the ple^{[ATG-,CC]} CopyCatcher element, the strong thorax and wing-specific MS1096-GAL4 driver, and UAS-RNAi cassettes (whose expression is induced by the GAL4 trans-activator) and scored the fraction of pale bristles in individuals carrying the UAS-RNAi construct relative to controls (FIG. 3A). As a control, we assessed the efficiency of an shRNA targeting the mCherry coding sequence. For each RNAi line tested (at least 3 independent crosses per RNAi line), we averaged the fraction of pale thoracic bristles in 15 control flies and in ≥10 flies of each RNAi genotype. We tabulated relative SGC frequencies (displayed as a heat map) by calculating the fold change of SGC events for each RNAi line relative to the batch mCherry RNAi controls (FIG. 3B and FIG. 3C). Among our four predicted positive RNAi lines, knock-down of Ku80 exhibited the strongest SGC-stimulating effects, with SGC increasing 1.75-fold over controls, while Irbp and DNA-lig4 knock-down demonstrated significant, but more modest, increases in SGC frequency (FIG. 3B and FIG. 3C). In contrast, down-regulation of Spn-A (Rad51) decreased the rate of SGC to 0.68-fold of control levels (FIG. 3B). These predicted effects of inhibiting known DSB repair components validated the CopyCatcher system as an effective in vivo genetic screening platform. Next, we screened the remaining 109 candidate genes implicated in DNA pairing and DSB repair, and identified Pros28.1 (1.64-fold), dm (1.62-fold), Orc1 (1.61-fold), eff (1.61-fold), and HP1c (1.58-fold) as loci inhibiting SGC (i.e., SGC was increased by RNAi of these genes) and βTub85D (0.36-fold), fs(1)h (0.52-fold), Np (0.53-fold), fzy (0.86-fold) and dup (0.85-fold) as promoters of SGC in Drosophila (i.e., SGC was decreased by RNAi of these genes, FIG. 3B, FIG. 3C, FIG. 3E)⁵⁴⁻⁶⁵.

Since RNAi typically results in reduction, but not elimination, of gene activity, we wondered whether simply reducing the gene dose of SGC candidate modifiers by 50% in heterozygous mutants might mimic the effect of RNAi. We confirmed that heterozygous loss-of-function alleles of the Np (0.65-fold), fzy (0.7-fold), βTub85D (0.62-fold) and fs(1)h (0.78-fold) loci decreased SGC frequency, while lowering the dosage of the eff (1.24-fold) and Orc1 (1.18-fold) genes resulted in enhanced SGC significantly (FIG. 3D). Another way to assess roles of candidate genes in promoting or antagonizing SGC is to overexpress them, which according to simple models would be predicted to have the opposite effect to knocking them down by RNAi (FIG. 3D). We found this was indeed to be the case regarding the HP1c (0.69-fold) and dm (0.67-fold) genes for which UAS-over-expression transgenes were available. Collectively, this analysis supports the use of CopyCatchers as useful tools to identify and evaluate new SGC modifiers in vivo.

### Functional Conservation of SGC Candidate Genes for DSB Repair in Human Cells

As indicated above, a significant bottleneck in applying CRISPR/Cas9 technologies to gene and cell therapies is the pronounced preference of somatic cells for repairing DSBs via NHEJ rather than HDR. We wondered whether human orthologs of genes modulating SGC in Drosophila would also influence rates of somatic HDR in human cells. For this purpose, we generated a fluorescent-based reporter system capable of simultaneously quantifying NHEJ and HDR events in HEK293T cells. This system consists of a stable human epithelial kidney cell-line expressing a single copy of a P2A-copGFP cassette inserted into the 3′ terminal region of the GAPDH gene (the second allele on the homolog chromosome carries an NHEJ-induced point mutation, which would be targeted by new gRNA^{NHEJ}). We could thus measure both plasmid and homolog chromosome-templated DSB repair using this heterozygous HEK293T GAPDH-copGFP cell line.

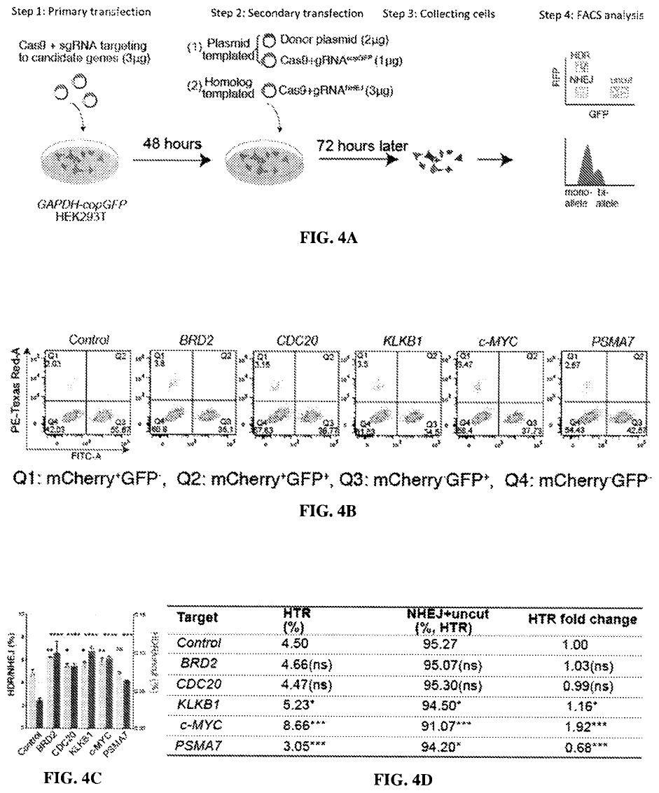

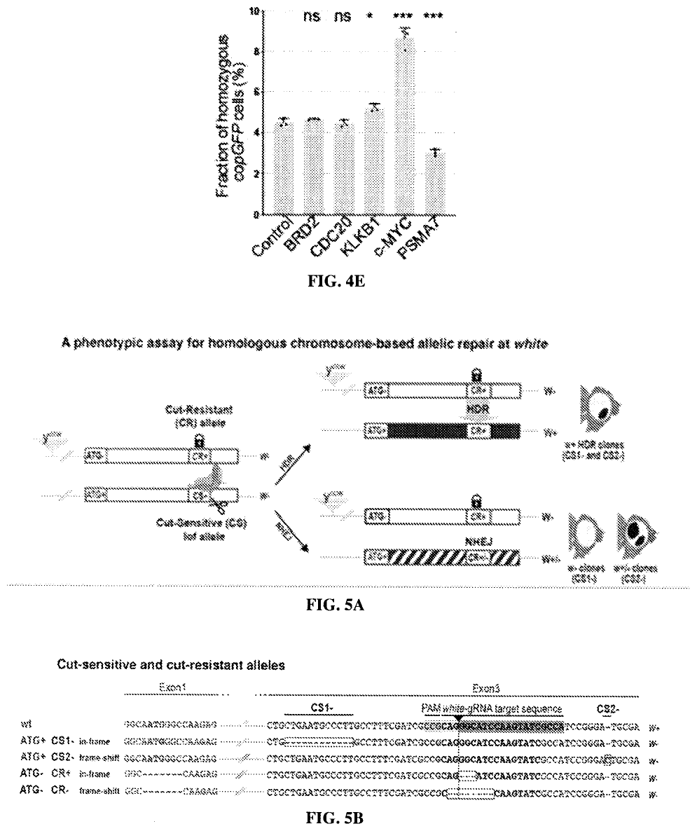

Plasmid-templated DSB repair was assayed by transfecting HEK293T GAPDH-copGFP cells with a combination of plasmids expressing SpCas9, a gRNA targeting to copGFP (gRNA^{copGFP}) and a promoter-less mCherry donor DNA template with homology arms flanking the copGFP gRNA cut site (FIG. 4A). In these traffic-light style experiments, loss of the GFP signal (Phase Q4 in FACS plots: GFP⁻ mCherry⁻) defines the fraction cells in which NHEJ events have mutated the GFP target gene, while concomitant gain of mCherry fluorescence (Phase Q1 in FACS plots: GFP⁻ mCherry⁺) reflects HDR events (FIG. 4B). Plasmids encoding Cas9 and gRNAs targeting candidate SGC modifiers were transfected into cells and 2-days later, we performed a second co-transfection with the mCherry donor plasmid and a plasmid encoding Cas9 and a gRNA targeting copGFP. After 72 hours, cells were harvested and analyzed by FACS (FIG. 4A). We tested the human homologs of the top 5 promoters or inhibitors of SGC identified in the fly RNAi screens. These human cell experiments confirmed that down-regulation of BRD2 (ortholog of Dmelfs(1)h), CDC20 (Dmelfzy), KLKB1 (DmelNP), c-MYC (Dmeldm) and PSMA7 (Dmelpros28.1) increased both NHEJ and HDR significantly (FIG. 3E, FIG. 4B). While there was a concordance of effects between the mammalian cells and Drosophila regarding the direction of effects on HDR and SGC for the c-MYC and PSMA7, we observed alterations of opposite sign to those identified in Drosophila for CDC20, BRD2 and KLKB1 (FIG. 4B and FIG. 4C). Notably, knock-down of c-MYC, resulting in approximately a 50% reduction in mRNA levels, increased the proportion of HDR-mediated fluorescence marker swapping relative to uncut GAPDH targets by 2.5-fold and increased the average ratio of HDR/NHEJ on average by 23% (FIG. 4B and FIG. 4C) This enhanced cassette copying could potentially be augmented further by more complete disruption of c-Myc expression. Thus, these pilot experiments identified inhibition of c-MYC as a new prime candidate for augmenting exogenous DNA-templated somatic HDR events in this system.

We conducted a further HDR analysis in the HEK293T GAPDH-copGFP cell line employing the homologous chromosome as the DSB repair template. In this system, no exogenous DNA template was provided. Instead, a gRNA targeting the NHEJ allele present on the homologous GAPDH allele was provided, thus creating a genome repair context similar to that of CopyCatchers in Drosophila. In this system, we scored the fraction of cells that were homozygous versus heterozygous for the GADPH-copGFP allele by quantitative fluorescence-activated cell sorting (FACS) that can distinguish cells carrying one versus two copies of GFP (see multi-level validation analysis summarized below). Using this mono- versus bi-allelic copGFP assay, we then knocked-down levels of the BRD2, CDC20, KLKB1, c-MYC and PSMA7 genes, all of which influenced rates of plasmid-templated HDR (FIG. 4B). Once again, c-MYC behaved as an SGC inhibitor as revealed by an increased rate of homolog-templated HDR (1.92-fold relative to controls, FIG. 4D and FIG. 4E). In addition, knocking-down KLKB1 and PSMA7 significantly altered the fraction of homozygous GFP, but these effects were opposite to those observed in Drosophila (FIG. 4D and FIG. 4E, compare to FIG. 3B and FIG. 3C).

The inferred genotypes of FACS sorted homozygous copGFP cells were verified by single cell cloning. We isolated single cell colonies from control (19 colonies) and c-MYC knock-down (17 colonies) treatments and verified the homozygosity of these isolated single colonies by amplifying the genome DNA with primers flanking the insertion site which could distinguish the longer insertion allele fragment from that of the shorter NHEJ allele. Only the longer copGFP bearing fragment was amplified from the bi-allelic GAPDH-copGFP cells, while amplification from a few mis-gated heterozygous GAPDH-copGFP cells revealed both long and short amplicon fragments. In validation of our stringent fluorescent gating protocol, 76% of the c-MYC knock-down clones (13 of 17 colonies) were verified as bi-allelic for the copGFP insertion, as were 74% (14 of 19 colonies) of the control colonies. Consistent with the inference from PCR analysis that clones amplifying only the single large band were homozygous for the copGFP element, the relative transcription level of copGFP was approximately double in these putative homozygous colonies compared to clones scored as being trans-heterozygous for the original copGFP and NHEJ allele.

As a further validation of cells interpreted as having undergone homolog-based repair with both chromosomes carrying the copGFP insertion of interest, we identified a closely linked polymorphism associated with the gRNA cleavage site (an SNP 94 bp from the cut site). We used primers from the copGFP element and adjacent genomic sequence to amplify and Sanger sequence a short fragment containing this SNP and the copGFP gene from inferred homozygous and heterozygous colonies. In three putative homozygous colonies (which also displayed elevated copGFP expression, colonies 2, 6 and 25), we observed equivalent double peaks at the SNP site, confirming that indeed both alleles were associated with the copGFP insertion. Similar analysis of three control heterozygous colonies (1, 9 and 23) revealed only a single peak, indicating that no HDR occurred in these colonies. In one homozygous clone (colony 34) we also observed a mono-allelic copGFP transgene. Given that the gRNA cut site was close to the SNP (94 bp), we imagine that in this clone localized gene conversion during the HDR repair process copied the donor polymorphism along with the copGFP element in this clone consistent with such islands of sequence conversion often extending 100-200 bp on either side of a DSB⁶⁶. In aggregate, these data strongly support the hypothesis that a great majority of the sorted cells we sorted as being homozygous do indeed carry two copies of the copGFP allele and that RNAi knock-down of the c-MYC gene increases the frequency of such events by nearly 2-fold.

### Discussion

In this study, we develop CopyCatchers as robust genetic systems for detecting and quantifying somatic gene conversion in Drosophila. Strategies for manipulating somatic DSB repair mechanisms have been extensively explored in mammalian cell lines, particularly with regard to treatments increasing the rate of HDR. Two frequently used systems are the DR-GFP (Direct-Repeat GFP) reporter and traffic-light switches⁶⁷⁻⁷⁰, which rely on either phenotypic or fluorescent outputs to score somatic HDR events. However, both of these reporters are difficult to adapt for high-throughput applications based on their high false-positive rates and complex repair outcomes^{68,69}. CopyCatchers offer an efficient alternative in vivo approach for detecting and quantifying somatic gene conversion events, and for therapeutic use.

### Design of CopyCatcher Systems

CopyCatchers are modified active genetic elements that incorporate several unique design features to serve as single-cell resolution reporters of HDR-mediated copying events in somatic cells^{2,71}. These recording elements are inserted into introns such that localized NHEJ induced indels are expected to be without phenotypic effect, excluding potential false positive signals, consistent with the observed strict concordance between fluorescent reporter and targeted loss-of-function phenotypes. DNA deep sequencing confirmed that the NHEJ events disrupt sequences at varying distances from the gRNA cut site, but do not extend into adjacent exons. CopyCatchers can carry a highly sensitive fluorescent reporter gene DsRed fused in-frame with targeted genes whose translation was abrogated by associated 5′ translation disruptive mutations. Thus, DsRed expression can only be recovered if a CopyCatcher element copies itself onto a wild-type homolog chromosome thereby separating itself from the 5′ mutation. We note that the CopyCatcher insertion sites were chosen to be sufficiently distant from the 5′ mutations to preclude co-copying with the CopyCatcher allele. In addition to reanimating DsRed expression, copying events also generate bi-allelic mutant cells, the clonal mitotic descendants of which all displayed concordant DsRed⁺ and homozygous loss-of-function phenotypes of the target gene.

### CopyCatchers Reveal Unexpectedly High Rates of Somatic Gene Conversion

The most striking result revealed by CopyCatchers is their unexpectedly high rate of SGC events in the white and ple loci. Qualitative (w^{[ATG-,CC]}) and quantitative (ple^{[ATG-,CC]}) assessments of SGC frequencies were in the range of 30-50% of cells in the targeted tissues (eye and thorax respectively). These observations suggest that a substantial fraction of somatic cell phenotypes produced by gene-drive systems in which the Cas9 activity is not strictly limited to the germline are likely to be caused by homozygosity of the drive element^{1,2,12}. Furthermore, by testing a variety of crossing schemes and promoters driving Cas9 expression, we identified particularly favorable configurations for promoting SGC (e.g., paternal transmission of either broadly expressed Cas9 alone or together with the CopyCatcher), which could be enhanced yet further by altering the activities of several genes associated with somatic DSB repair (see more detailed discussion below). Thus, far from being inefficient, HDR-mediated SGC employing the homologous chromosome as a correction template can serve as a frequent repair pathway in somatic cells of Drosophila. CopyCatcher elements also displayed efficient germline transmission. These quantitative and single-cell resolution findings are consistent with prior reports of frequent I-SceI-induced repair of a mini-white transgene using neighboring sequences located in-cis on the same chromosome in the germline^{72,73}, and with qualitative evidence for modest levels of bulk targeted cleavage noted in the soma^{72,73,74}, although it remains unclear whether there might be mechanistic differences distinguishing cis- versus homolog-templated repair⁷⁵. We offer the term Homologous Chromosome-Templated Repair (HTR) to refer to the mechanism underlying such SGC events.

### Relevance of SGC to Human Cell Engineering

Because mammalian chromosomes do not typically engage in inter-homolog pairing as pervasively as Drosophila, a potential concern could be that the high efficiency of HTR-driven SGC we observed in Drosophila would prove less efficient in somatic cells of other organisms in which chromosome pairing is less prominent^{52,76-77}. We note, however, that there is evidence in mammalian cells that the absence of chromosome pairing is due in part to an active anti-pairing process, suggesting that these cells too might be induced to engage in pairing by interfering with this suppressive process²⁴. Furthermore, homologous chromosome segments are actively recruited to DSBs located in transcribed regions of the genome during diploid phases of the cell cycle, indicating that HTR actively contributes to maintain cell viability^{79,80}. Indeed, in some cancer cell lines, specific chromosome arms have been found to be consistently paired along their length^{24,79,80}. Also, in loss of heterozygosity (LOH) mutants, the frequency of repairing DSB by copying from the homologous chromosome is greatly elevated^{27,28}. As discussed further below, results reported in this study indicate that HTR can indeed be detected in a human cell line and that novel factors influencing such SGC events in Drosophila (e.g., c-MYC) can similarly modify HTR in the human cell model.

The potential concerns discussed above regarding the potential role of chromosome pairing in mammalian cells notwithstanding, there is a great need to overcome inefficient Cas9-based genome editing in human cells since this technical impediment hampers development of potential therapeutic tools for treating human somatic diseases^{81,82}. To tackle this critical problem, various studies have pursued strategies of: inhibiting core components of the NHEJ machinery^{39,83-85}; stimulating the HDR pathway^{86,87}; synchronizing the cell cycle⁸⁸; concentrating DSB repair templates at the cutting site⁸⁹; or manipulating DSBs repair pathways in favor of HDR over NHEJ⁹⁰.

Because CopyCatchers provide exceedingly sensitive readouts for SGC, we tested whether they might also serve as tools to probe the genetic requirements for biasing somatic cell DSB repair in favor of HDR. In a pilot genetic screen of 109 factors either being essential for DNA pairing or associating with known DSB repair factors, we identified Orc1, HP1c, eff, Pros28.1 and dm as loci inhibiting SGC and βTub85D, fzy, Np, dup and fs(1)h as promoters of SGC in Drosophila. Among these candidates, we found c-MYC, the human ortholog of Drosophila dm, functions as SGC inhibitor during both plasmid and HTR in mammalian HEK293T cells, while BRD2, CDC20 and KLKB1 were identified as enhancers of SGC, confirming that CopyCatchers can serve as preliminary screening tools for genetic modifiers of SGC. These results suggest that the newly identified components modulating SGC in flies (dm), are also relevant to both plasmid and HTR in mammalian cells, although the details of how they do so may differ between systems since in some cases we observed opposite effects of SGC between flies and mammalian cells, which may reflect distinct mechanisms or components acting in these repair processes. In future studies, it will be important to test these strategies for increasing SGC rates in additional mammalian cell lines and primary cells. Such investigations may identify additional new factors or pathways which can be manipulated to bias somatic DSB repair choice, as well as small molecules that could influence the choice DSB repair pathway in favor of HTR in somatic cells^{80,81}. Ultimately, these new HTR-based systems will provide strategies to devise new incarnations of precision gene therapy.

### Materials and Methods

### DNA Manipulations and Constructions of CopyCatcher Elements

One-step assembly with NEBuilder HiFi DNA Assembly Master Mix was used for cloning of CopyCatcher plasmids⁹¹. As depicted in FIG. 1A, left and right homologous arms for each locus flanking gRNA cleavage site were amplified from the w¹¹⁸ wild-type fly genomic DNA. The SA-T2A sequence was amplified from pBS-KS-attB2-SA-T2A-3XGal80-Hsp70 series plasmids (Addgene 62951 and 62952)⁹², gRNAs-expressing cassette targeting to the first intron of yellow, white and ple were assembled following online published protocols (CRISPR fly Design-www.crisprflydesign.org/) and mCerulean selection marker was amplified from Addgene 27795 plasmid. DsRed was put at the downstream of T2A to indicate the copying events by fluorescence. Following assembly, CopyCatcher plasmids were transformed into NEB 5-alpha chemical competent E. coli cell (NEB C2987). Positive plasmids verified by sequencing were purified with Qiagen Plasmid Midi Kit (#12191), mixed with pBS-Hsp70-Cas9 (Addgene 46294) at 500 ng/μl and 250 ng/μl each, and sent to Rainbow Transgenic Flies, Inc. for injection into w¹¹⁸ wild-type (y^{[CC]} and ple^{[CC]}) or Oregon-R (w^{[CC]}) embryos. By screening the CFP fluorescent eye-marker phenotype, male transformants carrying the y^{[CC]}, w^{[CC]} and ple^{[CC]} CopyCatcher elements were identified and followed by genomic insertion confirmation by flanking PCR¹⁹.

### Drosophila Stocks and Genetics

Fly stocks were raised on standard Drosophila food under 18° C. with a 12/12 hours day/night cycle and experimental flies were raised at 25° C. The y^{[ATG-,CC]}k, w^{[ATG-,CC]} and ple^{[ATG-,CC]} CopyCatcher flies with 5′ out-of-frame allele were created either by recombination with existing alleles (y^{[ATG-,CC]} with y^{[1]}) or injecting gRNAs expressing by pCFD3 vector targeting to the site near ATG translational initiation codons (Addgene 49410) into CopyCatcher flies (w^{[ATG-,CC]} and ple^{[ATG-,CC]}) Compound out-frame CopyCatcher alleles recovered among F₁ generation progeny were used as the donor chromosome in FIG. 1A. The y^{[ATG-,CC]} and w^{[ATG-,CC]} lines were isogenized and made into a homozygous stocks, while ple^{[ATG-,CC]} remained balanced with TM6 due to the lethality of null mutations in this locus. The distance between ATG⁻ and gRNA cutting sites were designed as 2594 bp, 1694 bp and 958 bp for y^{[ATG-,CC]}, w^{[ATG-,CC]} and ple^{[ATG-,CC]} respectively. These distances are sufficient to prevent detectable co-transmission of ATG⁻ alleles with CopyCatcher elements together from donor to receiver chromosome as a consequence of being captured by HDR-mediated localized gene conversion, which typically extends only 150-200 bp (maximum 1 kb) on either side of the DSB that is being repaired^{33,34,93}. The y^{[ATG-,CC]}, w^{[ATG-,CC]} and ple^{[ATG-,CC]} flies carrying CopyCatcher elements and ATG⁻ allele were combined in various configurations described in the text with actin-Cas9, vasa-Cas9 or nanos-Cas9 lines on the X chromosome (kindly provided by Valentino M Gantz)¹⁴, or vasa-Cas9 on the third chromosome (BDSC 51324). Fly stocks used for RNAi-based genetic screening were ordered from Bloomington Drosophila stock center. Flies were anesthetized and selected by phenotyping under a Zeiss Stemi 2000 fluorescence microscope for somatic and germline mediated gene-drive experiments.

### Genomic DNA Preparation

Single adult flies were used for genomic DNA preparation according to the manufacturer instructions of Qiagen DNeasy Blood & Tissue Kit⁹⁴. In briefly, flies were crushed using 49 μl lysis buffer with 1 mM EDTA, 10 mM Tris pH 8.2 and 25 mM NaCl, and added with 1 μl Proteinase K to a final concentration of 0.3 mg/ml after the homogenization. Then the reaction mixture was incubated for 37° C. for 30 min, and 95° C. for 2 min. Samples were diluted with 150 μl ddH₂O and stored in −20° C.

### Screening for Genetic Modifiers of CopyCatcher Activity

A three-step crossing scheme was employed to screen for genes biasing the frequency of CopyCatcher induced SGC events. In briefly, we recombined the UAS-Cas9 (BDSC 54595) and ple^{[ATG-,CC]} on the third chromosome to generate ple^{[ATG-,CC]}-UAS-Cas9/TM6 firstly, and created a MS1096-GAL4; CyO/Sco or MS1096-GAL4; TM6/Sb stock. We then combined Gal4 and RNAi lines (MS1096-GAL4; UAS-RNAi, as well as targeted mutation lines or UAS over-expressing lines)⁵¹ according to the insertion site of the shRNA expressing cassettes. Finally, the ple^{[ATG-,CC]}-UAS-Cas9/TM6 virgins were crossed with males carrying MS1096-GAL4 and RNAi cassette, and all female progeny were used to score SGC.

### Generation of HEK293T Cell-Based Screening System for Quantitation of NHEJ and HDR Events

We generated a fluorescent-based reporter system in human cells by first inserting a P2A-copGFP (HEK293T GAPDH-copGFP cell line) sequence just before the stop codon of GAPDH and isolated a clone carrying one copy insertion by FACS. In addition, a point mutation was generated by NHEJ at the homologous chromosome at the same locus of gRNA targeted, creating a new gRNA targeting site and we termed it as NHEJ allele. We used this cell line for assessing two kinds of somatic HDR events: (1) Plasmid-templated HDR efficiency, by transfection plasmid expressing gRNA targeting to the copGFP (gRNA^{copGFP}: 5′-CTTCCTCTTGTGCTCTTGCTGGG-3′ (SEQ ID NO: 1)) as well as a donor DNA plasmid containing promoter-less mCherry sequence flanking the copGFP cut site. HDR efficiency was scored by the fraction of cells which are GFP⁻mCherry⁺. (2) Homologous chromosome templated somatic HDR, by transfection gRNA expression plasmid targeting to (gRNA^{NHEJ}: 5′-GCCCCAGCAAGAGCACCAAGAGG-3′ (SEQ ID NO: 2)) the NHEJ allele at the GAPDH locus. In this situation, HDR frequency was calculated by single or double copy expression of GFP. We evaluated the effect of candidate HDR modulators, which we screened in Drosophila in vivo, in altering the efficiency of HDR, first, by transfecting monoallelic copGFP expressing cells with guide RNAs targeting each candidate along with spCas9 and confirmed the mutation at the specified locus by Sanger sequencing and consequent reduction in mRNA by qRT-PCR. 48-hours after the first transfection, monoallelic copGFP cells were subjected to second round of transfection with gRNA^{copGFP} and donor DNA plasmid or gRNA^{NHEJ} only. Samples were harvested at 72 hours after transfection, washed with PBS, diluted in FACS buffer (2% FBS, 2 mM EDTA and 2 mM NaN₃ in PBS) and sent for FACS analysis.

### Amplicon-Based Deep Sequencing

Twenty flies of indicated genotype were collected for genomic DNA extraction. Genomic loci spanning the gRNA targets were PCR amplified with gene-specific primers added with 5′ tails complementary to the Trueseq adaptors (5′-ACACTCTTTCCCTACACGACGCTCTTCCGATCT-3′ (SEQ ID NO: 3) adaptors for forward primers and 5′-GACTGGAGTTCAGACGTGTGCTCTTCCGATCT-3′ (SEQ ID NO: 4) for reverse primers). Next, 5 μl of the amplification products were used for the secondary PCR with index-containing primers. The individual PCR products from different crosses were pooled together at the concentration of 10 nM for each sample and subjected to Illumina paired-end 100 bp Amplicon-based deep sequencing. All reads were analyzed with Bowite2 (http://bowtie-bio.sourceforge.net/bowtie2/index.shtml), and wild-type white and pale sequence were used for read counts extraction.

### Single Cell Colony

We verified our ability to distinguish cells heterozygous versus homozygous for the GADH-GFP insertion element in cell populations separated by FACS. We sorted single cells into individual wells of 96-well plates and grew them for approximately 2 weeks without changing medium until colonies could easily be observed. A set of primers targeting to sequences flanking the insertion cassette were used to PCR amplify either heterozygous or homozygous cop GFP. Two bands were amplified from the heterozygous GAPDH-copGFP cell line, one is 1025 bp (copGFP allele) and another 265 bp (NHEJ allele). These heterozygous cells were used as a negative control for copying of the GAPDH-GFP element. A homozygous GAPDH-copGFP line we had also derived, which was bi-allelic for copGFP (1025 bp), was used as the positive control for SGC in cells that started off in a heterozygous condition. We further validated homozygosity the single cell colonies by copGFP-specific amplification, and then definitively genotyped heterozygous versus homozygous GAPDH-copGFP cell lines using a single nucleotide polymorphism that distinguishes the copGFP and NHEJ alleles.

### Statistical Analysis

All the statistical analysis was performed with GraphPad Prism 7 by two-way ANOVA. Error bars in figures centered around the mean represent the standard deviation (±S.D.), and p-values (e.g., p<0.001) were used to affirm significance.

### Data Availability

The sequences of all plasmids used in this study has been deposited into GenBank Database with the accession number as following:

yellow CopyCatcher donor plasmid: MW770349

white CopyCatcher donor plasmid: MW770350

ple CopyCatcher donor plasmid: MW770351

mCherry donor plasmid: MW770352

### REFERENCES

1. Gantz, V. M. & Bier, E. The dawn of active genetics. Bioessays 38, 50-63 (2016).
2. Gantz, V. M. & Bier, E. Genome editing. The mutagenic chain reaction: A method for converting heterozygous to homozygous mutations. Science 348, 442-444 (2015).
3. Kyrou, K. et al. A CRISPR-Cas9 gene drive targeting doublesex causes complete population suppression in caged Anopheles gambiae mosquitoes. Nat. Biotechnol. 36, 1062-1066 (2018).
4. Limbo, O. et al. Ctp1 is a cell-cycle-regulated protein that functions with Mre11 complex to control double-strand break repair by homologous recombination. Mol. Cell 28, 134-146 (2007).
5. Sung, P. & Klein, H. Mechanism of homologous recombination: Mediators and helicases take on regulatory functions. Nat. Rev. Mol. Cell Biol. 7, 739-750 (2006).
6. Takata, M. et al. Homologous recombination and non-homologous end-joining pathways of DNA double-strand break repair have overlapping roles in the maintenance of chromosomal integrity in vertebrate cells. EMBO J. 17, 5497-5508 (1998).
7. Mao, Z. et al. Comparison of nonhomologous end joining and homologous recombination in human cells. DNA Repair (Amst) 7, 1765-1771 (2008).
8. Davis, A. J. & Chen, D. J. DNA double strand break repair via non-homologous end-joining. Transl. Cancer Res. 2, 130-143 (2013).
9. Pannunzio, N. R., Watanabe, G. & Lieber, M. R. Nonhomologous DNA end-joining for repair of DNA double-strand breaks. J. Biol. Chem. 293, 10512-10523 (2018).
10. Lieber, M. R. The Mechanism of double-Strand DNA break repair by the nonhomologous DNA end-Joining pathway. Annu. Rev. Biochem. 79, 181-211 (2010).
11. Bier, E. et al. Advances in engineering the fly genome with the CRISPR-Cas system. Genetics 208, 1-18 (2018).
12. Adolf, A. et al. Efficient population modification gene-drive rescue system in the malaria mosquito Anopheles stephensi. Nat. Commun. 11, 5553 (2020).
13. Hammond, A. et al. Regulating the expression of gene drives is key to increasing their invasive potential and the mitigation of resistance. PLoS Gent. 17, e1009321 (2021).
14. Carballar-Lejarazu, R. et al. Next-generation gene drive for population modification of the malaria vector mosquito, Anopheles gambiae. Proc. Natl. Acad. Sci. U.S.A. 117, 22805-22814 (2020).
15. Terradas, G., Buchman, A. B., Bennett, J. B. et al. Inherently confinable split-drive systems in Drosophila. Nat. Commun. 12, 1480 (2021).
16. Beaghton, A. K. et al. Gene drive for population genetic control: non-functional resistance and parental effects. Proc. Biol. Sci. 286, 20191586 (2019).
17. Grunwald, H. A. et al. Super-Mendelian inheritance mediated by CRISPR-Cas9 in the female mouse germline. Nature 566, 105-109 (2019).
18. Lopez, D. A. V. et al. A transcomplementing gene drive provides a flexible platform for laboratory investigation and potential field deployment. Nat. Commun. 11. 352 (2020).
19. Guichard, A. et al. Efficient allelic-drive in Drosophila. Nat. Commun. 10, 1640 (2019).
20. Hammond, A. et al. A CRISPR-Cas9 gene drive system targeting female reproduction in the malaria mosquito vector Anopheles gambiae. Nat. Biotechnol. 34, 78-83 (2016).
21. Li, M. et al. Development of a confinable gene drive system in the human disease vector Aedes aegypti. Elife. 9, e51701 (2020).
22. Lin, C. C. & Potter, C. J. Non-mendelian dominant maternal effects caused by CRISPR/Cas9 transgenic components in Drosophila melanogaster. G3 (Bethesda) 6, 3685-3691 (2016).
23. Champer, J. et al. Novel CRISPR/Cas9 gene drive constructs reveal insights into mechanisms of resistance allele formation and drive efficiency in genetically diverse populations. PLoS Genet. 13, e1006796 (2017).
24. Joyce, E. F., Erceg, J. & Wu, C. T. Pairing and anti-pairing: A balancing act in the diploid genome. Curr. Opin. Genet. Dev. 37, 119-128 (2016).
25. Kadyk, L. C. & Hartwell, L. H. Sister cromatids are preferred over homologs as substrates for recombinational repair in Saccharomyces cerevisiae. Genetics 132. 387-402 (1992).
26. Haber, J. E. TOPping off meiosis. Mol. Cell 57, 577-581 (2015).
27. Prakash, R. et al. Homologous recombination and human health: the roles of BRAC1, BRAC2 and associated proteins. Cold Spring Harb Perspect Biol. 7, a016600 (2015).
28. Ceccaldi, R., Rondinelli, B. & D'Andrea, A. D. Repair pathway choices and consequences at the double-strand break. Trends Cell Biol. 26, 52-64 (2016).
29. Goedecke, W. et al. Mre11 and Ku70 interact in somatic cells, but are differentially expressed in early meiosis. Nat. Genet. 23, 194-198 (1999).
30. Yoo, S. & McKee, B. D. Functional analysis of the Drosophila Rad51 gene (spn-A) in repair of DNA damage and meiotic chromosome segregation. DNA Repair (Amst). 4, 231-242 (2005).
31. Buis, J. et al. Mre11 regulates CtIP-dependent double-strand break repair by interaction with CDK2. Nat. Struct. Mol. Biol. 19, 246-252 (2012).
32. Lee-Theilen, M. et al. CtIP promotes microhomology -mediated alternative end-joining during class switch recombination. Nat. Struct. Mol. Biol. 18, 75-79 (2011).
33. Sartori, A. A. et al. Human CtIP promotes DNA end resection. Nature 450, 509-514 (2007).
34. Penkner, A. et al. A conserved function for a Caenorhabditis elegans Com1/Sae2/CtIP protein homolog in meiotic recombination. EMBO J. 26, 5071-5082 (2007).
35. Uckelmann, M. & Sixma, T. K. Histone ubiquitination in the DNA damage response. DNA Repair (Amst) 56, 92-101 (2017).
36. Enguita-Marruedo, A. et al. Transition from a meiotic to a somatic-like DNA damage response during the pachytene stage in mouse meiosis. PLoS Genet. 15, e1007439 (2019).
37. Zakharyevich, K. et al. Temporally and biochemically distinct activities of Exo1 during meiosis: double-strand break resection and resolution of double holiday junctions. Mol. Cell 40, 1001-1015 (2010).
38. Hodgson, A. et al. Mre11 and Exo1 contribute to the initiation and processivity of resection at meiotic double-strand breaks made independently of Spo11. DNA Repair (Amst) 10, 138-148 (2011).
39. Boersma, V. et al. MAD2L2 controls DNA repair at telomeres and DNA breaks by inhibiting 5′ end resection. Nature 521, 537-540 (2015).
40. Yin, Y. & Petes, D. T. The role of Exo1p exonuclease in DNA end resection to generate gene conversion tracts in Saccharomyces cerevisiae. Genetics, 197, 1097-1109 (2014).
41. Krishna, S. Mre11 and Ku regulation of double-stranded break repair by gene conversion and break-induced replication. DNA repair, 3, 2024-2030 (2006).
42. Weinert, B. & Rio, D. DNA strand displacement, strand annealing and strand swapping by the Drosophila Blooms syndrome helicase. Nucleic Acids Res. 35, 1367-1376 (2007).
43. McVey, M. et al. Formation of deletions during double-stranded break repair in Drosophila DmBlm mutants occurs after strand invasion. Proc. Natl. Acad. Res. 101, 15694-15699 (2004).
44. Maloisel, L., Fabre, F. & Ganfloff, S. DNA polymerase δ is preferentially recruited during homologous recombination to promote heteroduplex DNA extension. Mol. Cell Biol. 28, 1373-1382 (2008).
45. Spies, M. & Fishel, R. Mismatch repair during homologous and hemeologous recombination. DNA Repair, 38, 75-83 (2016).
46. Eetl, H. et al. The role of Blm helicase in homologous recombination, gene conversion tract length, and recombination between diverged sequences in Drosophila melanogaster. Genetics, 207, 923-933 (2017).
47. Patton, J. S., Gomes, X. V. & Geyer, P. K. Position-independent germline transformation in Drosophila using a cuticle pigmentation gene as a selectable marker. Nucleic Acids Res. 20, 5859-5860 (1992).
48. Wanger, C. R., Mahowald, A. P. & Miller, K. G. One of the two cytoplasmic actin isoforms in Drosophila is essential. Proc. Natl. Acad. Sci. U.S.A. 99, 8037-8042 (2002).
49. Kevin, M. F. & Elizabeth, R. G. Live imaging of endogenous RNA reveals a diffusion and extrapment mechanism for nanos mRNA localization in Drosophila. Curr Biol. 13, 1159-1168 (2003).
50. Andrew, D. R. Vasa is expressed in somatic cells of the embryonic gonad in a sex-specific manner in Drosophila melanogaster. Biol. Open. 1, 1043-1048 (2012).
51. Ewen-Campen, B. et al. Optimized strategy for in vivo Cas9-activation in Drosophila. Proc. Natl. Acad. Sci. U.S.A. 114, 9409-9414 (2017).
52. Joyce, E. F. et al. Identification of genes that promote or antagonize somatic homolog pairing using a high-throughput FISH-based screen. PLoS Genet. 8. e1002667 (2012).
53. Brand, A. H. & Perrimon, N. Targeted gene expression as a means of altering cell fates and generating dominant phenotypes. Development 118. 401-415 (1993).
54. Passemard, S., Kaindl, M. A. & Verloes, A. Microcephaly. Handb. Clin. Neurol. 111. 129-141 (Elsevier Press, Paris, 2013).
55. Gursoy-Yuzugullu, O., Carman, C. & Price, B. D. Spatially restricted loading of BRD2 at DNA double-stranded breaks protects H4 acetylation domains and promotes DNA repair. Sci. Rep. 7. 12921 (2017).
56. Feener, P. E., Zhou, Q. & Fickweiler, W. Role of plasma kallikrein in diabetes and metabolism. Thromb. Haemost. 110. 434-441 (2013).
57. Connors, B. et al. A systemic interaction between CDC20 and RAD4 in Saccharomyces cerevisiae upon UV irradiation. Mol. Biol. Int. 2014. 519290 (2014).
58. Rodgers, K. & McVey, M. Error-prone of DNA double-stranded breaks. J. Cell Physiol. 231. 15-24 (2016).
59. Dinant, C. & Luijsterburg, M. S. The emerging role of HP1 in the DNA damage response. Mol. Cell. Biol. 29. 6335-6340 (2009).
60. Schmidt, C. et al. Systematic E2 screening reveals a UBE2D-RNF138-CtIP axis promoting DNA repair. Nat. Cell Biol. 17. 1458-1470 (2015).
61. De loannes, P., et al. Structure and function of the Orc1 BAH-nucleosome complex. Nat. Commun. 10, 2894 (2019).
62. Luoto, K. R. et al. Tumor cell kill by c-MYC depletion: role of MYC-regulated genes that control DNA double-strand break repair. Cancer Res. 70. 8748-8759 (2010).
63. Muvarak, N. et al. C-MYC generates repair errors via increased transcription of alternative-NHEJ factors, LIG3 and PARP1, in tyrosine kinase-activated leukemias. Mol. Cancer Res. 13. 669-712 (2015).
64. Fernandez-Diez, C. et al. Inhibition of zygotic DNA repair: transcriptome analysis of the off spring in trout (Oncorhynchus mykiss). Reproduction 149. 101-111 (2015).
65. Gomez-H, L. et al. The PSMA8 subunit of the spermatoproteasome is essential for proper meiotic exit and mouse fertility. PLoS Genet. 15. e1008316 (2019).
66. Zhou. Y. et. al. Quantitation of DNA double-strand break resection intermediates in human cells. Nucleic Acids Res. 42. e19 (2019).
67. Rong, Y. S. & Golic, K. G. Gene targeting by homologous recombination in Drosophila. Science 288, 2013-2018 (2000).
68. Kass, E. M. et al. Double-strand break repair by homologous recombination in primary mouse somatic cells requires BRCA1 but not the ATM kinase. Proc. Natl. Acad. Sci. U.S.A. 110, 5564-5569 (2013).
69. Aksoy, Y. A. et al. Chemical reprogramming enhances homology-directed genome editing in zebrafish embryos. Commun. Biol. 2, 198 (2019).
70. Certo, M. T. Tracking genome engineering outcome at individual DNA breakpoints. Nat. Methods 8, 671-676 (2011).
71. Xu, S. et al. CRISPR/Cas9 and active genetics-based trans-species replacement of the endogenous Drosophila kni-L2 CRM reveals unexpected complexity. Elife 6, e30281 (2017).
72. Do, A. T. et al. Double-strand break repair assays determine pathway choice and structure of gene conversion events in Drosophila melanogaster. G3 (Bethesda) 4, 425-432(2014).
73. Wei, D. S. & Rong, Y. S. A genetic screen for DNA double-strand break repair mutations in Drosophila. Genetics 177, 63-77 (2007).
74. Rong, Y. S & Golic, K. G. The homologous chromosome is an effective template for the repair of mitotic DNA double-stranded breaks in Drosophila. Genetics 165, 1831-1842 (2003).
75. Fernandez, J. et al. Chromosome preference during homologous recombination Repair of DNA double-strand breaks in Drosophila melanogaster. G3 (Bethesda) 9, 2019. 3773-3780.
76. Lewis, E. B. The theory and application of a new method of detecting chromosomal rearrangements in Drosophila melanogaster. Amer. Naturalist 88, 225-239 (1954).
77. Metz, C. W. Chromosome studies on the Diptera. II. The paired association of chromosomes in the Diptera, and its significance. J. Exp. Zool. 21, 213-280 (1916).
78. McKee, B. D. Homologous pairing and chromosome dynamics in meiosis and mitosis. Biochim. Biophys. Acta. 15. 165-180 (2004).
79. Gandhi, M. et al. Homologous chromosome make contact at the sites of double-strand breaks in genes in somatic GO/G1-phase human cells. Proc. Natl. Acad. Sci. U.S.A. 109, 9454-9459 (2012).
80. Gandhi, M. et al. Homologous chromosomes move and rapidly initiate contact at the sites of double-strand breaks in genes in GO-phase human cells. Cell Cycle 12, 547-552 (2013).
81. Hartlerode, A. J. & Scully, R. Mechanisms of double-strand break repair in somatic mammalian cells. Biochem. J. 423, 157-168 (2009).
82. Wright, W. D., Shah, S. S., & Heyer, W. D. Homologous recombination and the repair of DNA double-strand breaks. J. Biol. Chem. 293, 10524-10535 (2018).
83. Maruyama, T. et al. Increasing the efficiency of precise genome editing with CRISPR-Cas9 by inhibition of nonhomologous end joining. Nat. Biotechnol. 33, 538-542 (2015).
84. Srivastava, M. et al. An inhibitor of nonhomologous end-joining abrogates double-strand break repair and impedes cancer progression. Cell 151, 1474-1487 (2012).
85. Robert, F. et al. Pharmacological inhibition of DNA-PK stimulates Cas9-mediated genome editing. Genome Med. 7, 93 (2015).
86. Tran, N. T. et al. Enhancement of precise gene editing by the association of Cas9 with homologous recombination factors. Front. Genet. 10, 365 (2019).
87. Charpentier, M. et al. CtIP fusion to Cas9 enhances transgene integration by homology-dependent repair. Nat. Commun. 9, 1133 (2018).
88. Lin, S. et al. Enhanced homology-directed human genome engineering by controlled timing of CRISPR/Cas9 delivery. Elife 3, e04766 (2014).
89. Aird, E. J. et al. Increasing Cas9-mediated homology-directed repair efficiency through covalent tethering of DNA repair template. Commun. Biol. 1, 54 (2018).
90. Nami, F. et al. Strategies for in vivo genome editing in nondividing cells. Trends Biotechnol. 36, 770-786 (2018).
91. Barra, V. et al. Phosphorylation of CENP-A on serine 7 does not control centromere function. Nat. Commun. 10, 175 (2019).
92. Diao, F. et al. Plug-and-play genetic access to Drosophila cell types using exchangeable exon cassettes. Cell Rep. 10, 1410-1421 (2015).
93. Keelagher, R. E. et al., Separable roles for exonuclease I in meiotic DNA double-strand breaks repair. DNA Repair (Amst) 10, 126-137 (2011).
94. Gloor, G. B. et al. Type I repressors of P element mobility. Genetics 135, 81-95 (1993).

### Example 2—Cas9/Nickase-Induced Allelic Conversion by Homologous Chromosome-Templated Repair in Drosophila Somatic Cells

### Introduction

CRISPR (Clustered Regularly Interspaced Short Palindromic Repeats) components from S. thermophilus were initially discovered in bacteria as a defense system against phage infection and invasion by foreign DNA (1). Components of this natural immunity pathway have been modified and repurposed to produce specific and effective DNA cleavage and subsequent gene-editing in eukaryotic cells for a myriad of applications in basic research, medicine, biotechnology, and agriculture (2-6). The Cas9 endonuclease, when paired with a chimeric guide RNA (sgRNA, or gRNA), cleaves DNA at a precise genomic site defined by the gRNA sequence. Resulting double-strand breaks (DSB) then can be repaired by the cellular machinery through several pathways, which can be divided into two major groups: the error-prone Non-Homologous End Joining (NHEJ) pathway, which operates to reconnect loose ends, and the more precise homology-directed repair (HDR) pathways, which use a homologous DNA template for directional gene conversion events (7). Thus, random mutations can be created at specific sites through NHEJ, while for other applications, a donor plasmid with cargo DNA flanked by homology arms can direct insertion of desired sequences at the site of cleavage through HDR processes (8-10).

CRISPR components have also been configured into so-called active genetic systems to favor inheritance of desired traits and potentially modify insect and other populations (11-13). Such systems consist of DNA cassettes encoding a gRNA targeting their exact site of insertion. In the germline of heterozygous animals, site-specific cleavage of the naïve chromosome results in copying of the gene-drive cassette onto the recipient chromosome, leading to its super-Mendelian transmission. As a consequence, “gene-drives” have the ability to spread rapidly through a targeted population, and can be used to modify or suppress insect vectors (14). Similar active genetic systems have also been developed successfully in mammalian models for a variety of applications (15). Additional components can be included in the inserted gene cassettes, such as visible markers or genes conferring resistance to vector-transmitted pathogens (11, 16).

A variation on the gene-drive principle termed “allelic-drive” includes an additional gRNA that targets a second locus, distinct from the site of gene-drive insertion (17). Such a gRNA can be designed in an allele-specific fashion, to promote dissemination of a cut-resistant allele at the expense of a cut-sensitive allele at the same site. A proof-of-principle for this strategy was conducted in Drosophila for the Notch locus (17). When combined with a Cas9-expressing transgene, the allelic-drive produced a high rate of allelic conversion resulting in super-Mendelian inheritance of the cut-resistant allele. This study also proposed a similar and equally efficient approach referred to as “copy-grafting” that favors transmission of an allelic variant located near (rather than at) a cut-resistant site. These allelic drives could be harnessed for a variety of practical applications, such as disseminating naturally occurring alleles that block parasite transmission by insect vectors (18) or eliminating mutations conferring insecticide resistance (19, 20).

Both gene-drives and allelic-drives rely on copying of genetic material from the homologous chromosome following Cas9-induced position-specific cleavage. This HDR-mediated DNA repair process is highly efficient in the germline but has generally been considered inoperative in somatic tissues (21). In a recent study, we challenged this premise by demonstrating that inter-homolog copying of multi-kilobase gene cassettes can take place with great efficiency in somatic cells of Drosophila (22). These transgenic cassettes referred to as CopyCatchers were designed to reveal such somatic gene conversion (SGC) events at several different loci. Upon targeted Cas9-dependent cleavage, CopyCatchers copied onto receiver chromosomes at surprisingly high frequencies (30-50%). Importantly, this process of Cas9-elicited inter-homolog repair could also be demonstrated in human cells (albeit with lower efficiency), and in mouse embryos with increased dosage of Rad51 (22, 23).

In the present example, we first provide an in-depth analysis of somatic Homologous Chromosome-Templated Repair (HTR) of mutant alleles of the white locus in Drosophila. The diverse combination of alleles tested revealed successful repair outcomes by HDR, NHEJ, or by combinations of these events through the production of red pigmented cell clones in an otherwise white-eyed mutant background. Next, we make the surprising discovery that Cas9 nickase variants D10A or H840A, which generate targeted single-strand breaks (SSB) rather than double-strand breaks (DSB), also elicit HTR, and do so at levels yet higher than those achieved by Cas9. As expected (24), D10A and H840A induced very few NHEJ mutations. This Cas9 and Nickase-elicited HTR strategies rely on introduction of few genetic components and harness the cellular machinery to revert a genetic alteration to a wild-type functional state using endogenous templates. HTR-based strategies may enable the development of alternative gene therapies, for correcting many dominant or trans-heterozygous disease-causing DNA alterations.

### Results

### Allele-Specific DNA Cleavage Induces Both HDR and NHEJ Repair Events

Previously characterized gene-drive cassettes sustaining allelic drive carried two gRNAs: one directing cleavage at their own site of insertion, and another targeting a specific allele of a separate locus. Upon introduction of a source of Cas9, the targeted allele is cleaved and replaced by homologous cut-resistant sequences in the germline (17).

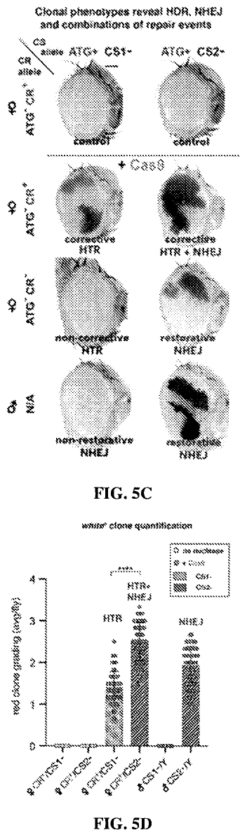

In the current study, we evaluate such interhomolog allelic-conversion at the X-linked white (w) locus in somatic cells. We designed a variety of configurations in which allele-specific DNA cleavage leads to phenotypically visible and quantifiable repair events resulting in restoration of red eye pigmentation attributable to either Homology-Directed Repair (HDR) or Non-Homologous End Joining (NHEJ) pathways (FIG. 5A). A previously validated transgenic y^{ccw} gene-drive element inserted in the yellow (y) locus, encodes two gRNAs, one (y-gRNA) targeting its own site of insertion (promoting initial integration and copying of the y^{ccw} cassette), and the other (white-gRNA) targeting cleavage in the third exon of the white gene, ˜1.5 centimorgans from yellow toward the centromere, (FIG. 5A, and (12)).

In addition, we created a set of “test” w⁻ alleles, with specific features allowing repair events to be visualized through the production of red (w⁺) eye clones in a w⁻ background. These w⁻ mutations were created through co-injection of either of two gRNA-expressing constructs (5′ gRNA or 3′ gRNA) with a transient source of Cas into white⁺ embryos. These w⁻ mutations lie in close proximity (25-30 nt) either 5′ or 3′ to the white-gRNA cut site (FIG. 1B), but remain sensitive to DNA cleavage (therefore termed cut-sensitive, or CS), and may be repaired either through HDR or NHEJ processes. Two alleles, CS1⁻ (an in-frame 12 nt deletion) and CS2⁻ (a 1 nt frame-shift insertion), were selected to conduct experiments described below (FIG. 5B). A second set of mutations was created by combining the y^{ccw} element and a source of Cas9 in w^{+/+} flies. DNA cleavage at w on both chromosomes followed by NHEJ repair generated either functional or non-functional cut-resistant (CR+ and CR⁻) alleles recovered in the F2 generation (see FIG. 5B for CR alleles recovered). Chromosomes carrying these mutations act as “donors”, since they are no longer sensitive to DNA cleavage by Cas9, but can provide homologous templates for repairing cleaved cognate alleles. Finally, we generated a w⁻ mutation deleting the ATG translation initiation site, which lies ˜3.7 kb upstream from the white-gRNA cut-site (FIG. 5B). This ATG⁻ mutation was combined with either of the cut-resistant CR⁺ or CR⁻ mutations and the y^{ccw} insertion (FIG. 5B) using Cas9-mediated allelic conversion. These latter combinations result in overall w⁻ donor chromosomes (y^{ccw} w^{ATG- CR+} and y^{ccw} w^{ATG- CR-}) to permit scoring of repair events that are expected to result in w⁺ clones in an otherwise w⁻ background (FIG. 5A).

We visualized Cas9-driven repair of the CS1⁻ allele by crossing w^{ATG+ CS1-}/Y; vasaCas9 males (providing Cas9 ubiquitously in somatic cells (25)) with y^{ccw} w^{ATG- CR+} females. F1 female progeny consistently displayed large patches of red ommatidia of varying sizes (see FIG. 5C and FIG. 5D for a range of graded phenotypes). These Cas9-dependent phenotypes indicate that DNA cleavage at the white-gRNA cut site occurred efficiently, presumably resulting in conversion of CS1⁻ to CR⁺, and copying the CR⁺ functional allele onto the homolog chromosome with the intact ATG⁺ start codon. We confirmed that these repair events were produced through Homologous chromosome-Templated Repair (HTR) by examining eyes from y^{ccw} w^{ATG- CR-}/w^{ATG+ CS1-}; vasaCas9/+ F1 females, in which the cut-resistant donor allele was non-functional (CR⁻). As expected, such animals had entirely white eyes (non-corrective HDR FIG. 5C). Since the outcome of the repair process for CS1− depends exclusively on the nature of the CR donor allele (CR+: red clones, or CR⁻: absence of red clones), we conclude that HTR is the exclusive process operating to produce w⁺ clones when the donor allele is functional (CR⁺). We also examined the progeny of y^{ccw} w^{ATG- CR+}/w^{ATG+ CS} females and observed high frequencies of w⁺ individuals (˜40%, close to the maximal theoretical 50% value), indicating that Cas9-dependent allelic conversion also was operating efficiently in the female germline to produce functional ATG⁺ CR⁺ alleles.

### Isolating Cas9-Induced NHEJ Events

To probe the activity of the NHEJ pathway in our system, we produced males in which the y^{ccw} insertion was combined with a CS⁻ allele in the presence of Cas9. In such hemizygous animals lacking a second X-chromosome, only NHEJ-based repair operates following DNA cleavage. We found indeed that NHEJ-based repair occurred in these animals, but that repair phenotypes depended on which CS⁻ allele was tested. While males carrying the CS1⁻ allele had entirely white eyes, cognate CS2⁻ animals exhibited numerous red clones (FIG. 5C bottom panels). Because the CS2⁻ allele is a frameshift mutation consisting of a one nucleotide (nt) insertion in proximity to the white-gRNA cut-site, we hypothesized that a fraction of NHEJ mutagenic events might restore the proper reading frame, potentially leading to functional w⁺ clones (restorative NHEJ). In contrast, the CS1⁻ allele is an in-frame 12 nt deletion eliminating four essential amino-acids. Such mutation is not amenable to frame restoration through mutagenic events produced by the NHEJ pathway, consistent with all flies having solid white eyes (FIG. 5C-5D). We confirmed the “frame restoration” hypothesis by sequencing individual w⁺ and w⁻ F2 progeny from y^{ccw} w^{ATG+ CS2-}/Y; Cas9/+ males. w⁺ F2 animals (representing about a third of the total progeny) consistently revealed DNA lesions restoring the correct reading frame, while lesions found in their w⁻ siblings did not.

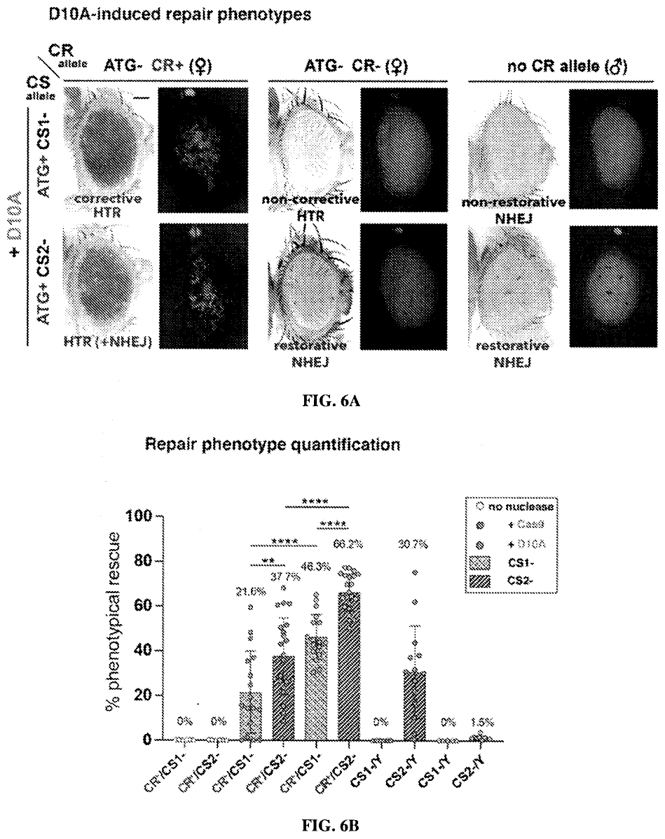

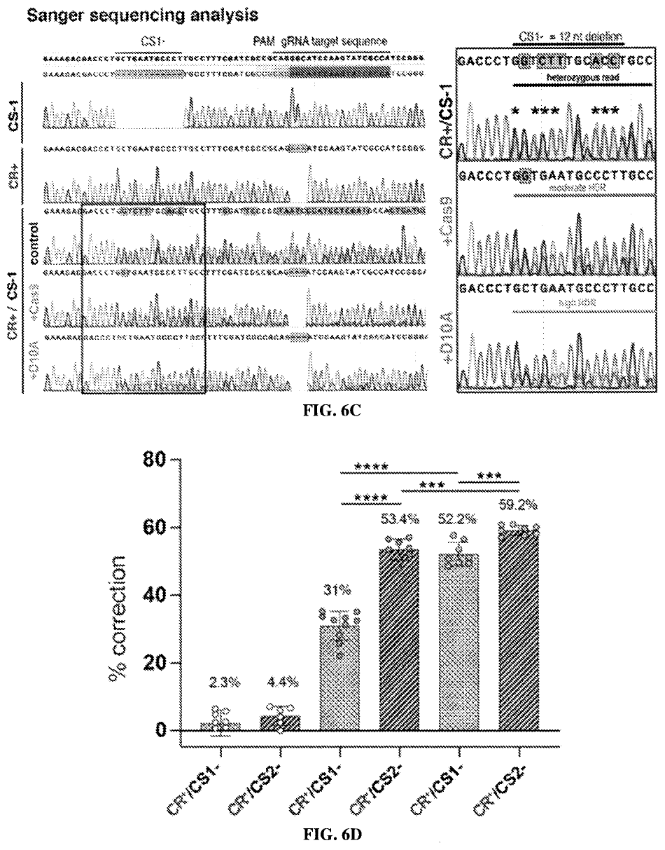

In summary, allele-specific DNA cleavage at w leads to visible and quantifiable red eye clones which can be attributed specifically either to HDR exploiting cut-resistant sequences from the homologous chromosome (in CR+/CS1⁻; Cas9/+ females), or to NHEJ (in CS2⁻/Y; Cas9/+ males). In addition, in females carrying the CS2⁻ allele (FIG. 5C, upper right panel), both HDR- and NHEJ-driven repair may lead to formation of w⁺ clones. Consistent with this latter inference, the incidence of w⁺ clones for the CS2⁻ allele was significantly higher than for the CS1⁻ allele, in which such w⁺ clones result exclusively from HDR (FIG. 5C and FIG. 6B).

### The D10A Nickase Supports Efficient Allelic Conversion

Cas9D10A nickase (abbreviated as D10A hereafter), which lacks activity of the RuvC catalytic domain (3), cuts only the targeted DNA strand (the strand hybridizing to the gRNA) to generate single SSBs. Nickases have been used successfully for gene editing in mammalian cells, using exogenous DNA repair templates. Although typically less efficient than Cas9 for such gene-editing, Nickases generate far fewer mutagenic events (24). We therefore wondered whether SSB could also promote HTR of a targeted allele. As in experiments described above, we produced y^{ccw} w^{ATG- CR+}/w^{ATG+ CS1-}; D10A/+ females. Surprisingly, these individuals manifested strong repair phenotypes, in which most of the eye surface appeared pigmented (FIG. 6A, top left panels). In contrast, y^{ccw} w^{ATG- CR-}/w^{ATG+ CS1-}; D10A/+ females carrying the non-functional CR⁻ donor allele displayed entirely white eyes (top middle panels), and only rare minute w⁺ clones for y^{ccw} w^{ATG- CR-}/w^{ATG+ CS2-}; D10A/+ animals (bottom middle panels), consistent with the expectation that the cleavage-resistant allele present on the homologous chromosome serves as the repair template. Unlike Cas9-induced w⁺ clones presented in FIG. 5C, which appeared as solid pigmented areas of varying sizes and shapes, D10A-induced clones were small and distributed uniformly in a high-density salt-and-pepper pattern across the surface of the eye for both CS1− and CS2− alleles (FIG. 6A, left panels).

The contrasting patterns of w⁺ clones induced by intact Cas9 and the D10A nickase indicate that D10A-elicited DNA repair occurs later during development but with possibly greater efficiency compared to Cas9-induced events. Examination of these mosaic eyes under a fluorescence microscope revealed the pattern of DNA repair with superior cellular resolution than could be achieved with bright field illumination, since GFP fluorescence (resulting from eye-specific expression of the 3XP3-GFP marker for the vasaD10A insertion) is readily visible in w⁻ areas, but is absorbed by eye pigments in w⁺ clones. Whereas Cas9-generated clones appear as large solid black areas, D10A-generated phenotypes appear as a dense array of small black clones scattered throughout the entire surface of the eye (FIG. 6A, left panels). The later developmental window for the generation of successful HDR events elicited by D10A versus Cas9 cannot be attributed to different expression profiles since both nucleases are expressed under the control of the same vasa promoter. Rather, the differing clonal rescue patterns most likely reflect distinct mechanisms and/or timing of the repair process.

### The D10A Nickase Induces Few NHEJ Events

DNA nicks can be readily repaired by ligation and rarely create NHEJ-mediated lesions. To test whether D10A had any mutagenic activity in our system, we made use of males in which the absence of a homologous X-chromosome allows assessment of the frequency with which NHEJ events restore w⁺ function in response to D10A-induced nicks for the CS2⁻ allele. As for Cas9, w⁺ clones in CS2/Y; D10A males were visible, but very small and rare (amounting to 8-25 small clones per eye), leaving most of the surface white (FIG. 6A, lower right panels). In contrast, no w⁺ clones were produced in y^{ccw} w^{ATG+ CS1-}/Y; D10A animals (FIG. 6A, upper right panels), consistent with the observation that the CS1⁻ allele cannot be restored to functionality by NHEJ mutagenic events (FIGS. 5C-5D).

D10A is More Efficient than Cas9 in Inducing Allelic Correction

Because of their contrasting patterns (large clonal sectors versus scattered small clones), Cas9- and D10A-induced HDR phenotypes are difficult to compare quantitatively. We resolved this difficulty by employing an image-based quantification method, in which analysis of multiple eye pictures acquired in the GFP channel allows global quantitation of pigmented areas and estimation of the overall repair percentage. This analysis revealed that D10A-induced SSB lead to a significantly greater percentage of correction (˜46%) than Cas9 (˜22%) with regard to correction of the CS1⁻ allele, for which only HDR events are able to restore the w⁺ function (FIG. 6B). Interestingly, D10A-elicited nicking of the CS2⁻ allele gave rise to an even higher percentage of repair (˜66%). This difference with the CS1⁻ allele cannot be attributed solely to a contribution of NHEJ repair, which only amounted to a low 1.5% repair in w^{ATG+ CS2-}/Y; D10A males (FIG. 6B). We conclude that differences in position (5′ versus 3′ to the cut site) as well as the nature (1 nt versus 12 nt alterations) between the CS1⁻ and CS2⁻ alleles are the determining factors responsible for their differing repair outcomes.

### Confirmatory Molecular Analysis of Cas9 Versus D10A Editing Events

We complemented our phenotypic assessment of allelic repair events by genomic sequence analysis of regions encompassing the white-gRNA cleavage site in individual flies where DNA cleavage was produced either by Cas9 or D10A. DNA sequence chromatograms from control heterozygous flies (CR+/CS1⁻) revealed the expected overlapping peaks of similar heights, starting precisely at the CS1⁻ 12 nt deletion breakpoint. In the presence of Cas9, peaks corresponding to the donor (CR⁺) sequences appeared consistently higher (FIG. 6C), at the expense of the receiver sequences (CS1⁻). Quantitative analysis of sequences from 6-10 independent reads revealed that correction increased from an average of ˜-2% in control flies to ˜30% in Cas9-expressing flies, consistent with the pigmentation analysis (FIG. 6B). In D10A-expressing flies, this enhancement was even more pronounced, amounting to an average of ˜52% correction, again indicating that repair following SSB results in more frequent HTR than observed for Cas9-induced DSB (FIG. 6D, Table 1). With regard to phenotypic quantification, correction of the CS2⁻ allele was even more efficient than for CS1⁻, amounting to an average of 53% for Cas9 and 59% for D10A. The three nt deletion at the cleavage site present in the donor allele also dominated in chromatograms from CR⁺/CS1⁻; D10A individuals, as is expected from a high proportion of HDR events. This feature was challenging to discern in CR⁺/CS1⁻; Cas9 animals, in which NHEJ events presumably concealed this effect. These results also demonstrate that HTR occurs throughout the developing body and is not restricted to the eye tissue in which the w gene is expressed. In contrast to these results in somatic tissues, we found that D10A was not inducing efficient HTR in the germline, suggesting that specific somatic factors/processes may determine the success of Nickase-induced HTR.

TABLE 1
Comparative outcomes of allelic repair induced by Cas9 and D10A

| | | Germline | | | | Repair | |
|---|---|---|---|---|---|---|---|
| Nuclease: | | repair | | | Multi Kb | after bi- | Pairing- |
| type of | Somatic | visible | | Developmental | insertion | allelic | independent |
| cleavage | HTR | in F2 | NHEJ | timing | copying | cleavage | repair |
| Cas9: | Yes, | High | High | Early | Yes, | No or | Not |
| DSB | moderate | levels | levels | (large | moderate | very | detected |
| | | | | clones) | | little | |
| D10A: | Yes, | Very | Very | Late (small | Low | Yes, | Yes, |
| SSB | efficient | low | low | clones) | levels | efficient | moderate |
| | | levels | levels | | | | |

### Comparative Analysis of D10A and H840A Repair Outcomes

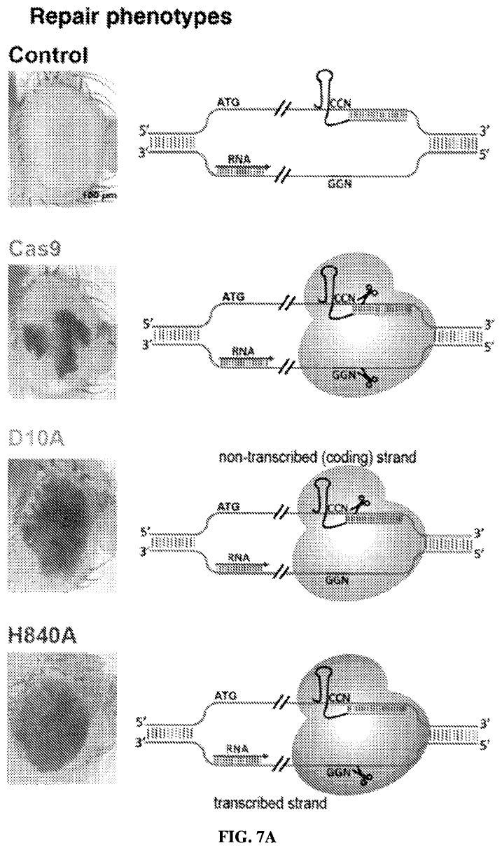

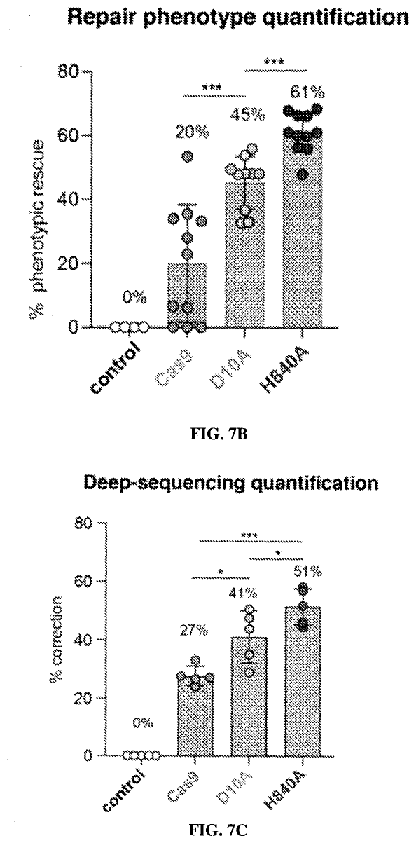

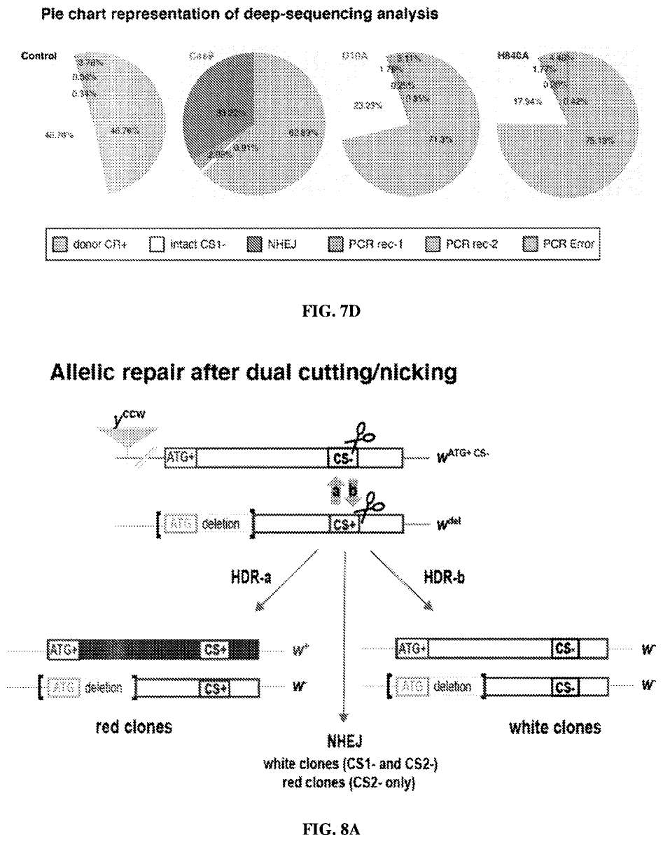

We took our analysis further by comparing the relative efficiencies of HTR processes following DSB or nicks targeting opposing DNA strands. For these experiments, we made use of three equivalent transgenic cassettes inserted at the same site to express either Cas9, D10A, or the alternate H840A nickase at identical levels to repair the CS1⁻ allele (26). H840A is mutated in the HNH catalytic domain and thus produces SSB on the opposite non-targeted DNA strand from that cleaved by D10A. Pigmentation phenotypes revealed that H840A also produced HTR phenotypes similar in pattern to those of D10A. H840A however, was consistently more efficient than D10A in restoring w⁺ gene function of the CS1⁻ allele (61% and 45% respectively), while Cas9 again produced lower percentages of correction (20%, FIG. 7A and FIG. 7B). Consistent with this phenotypic assessment, deep sequencing analysis revealed that the H840A nickase, which cleaves the transcribed strand in our system, led to significantly higher HDR levels than the D10A variant (51% for H840A and for 41% D10A, the latter targeting the non-transcribed coding strand, FIG. 7C). As observed previously, Cas9 cleavage generated a large proportion of NHEJ mutagenic events (˜33%) in addition to HDR-mediated allelic repair for the CS1⁻ allele (˜27%, FIGS. 7C-7D). Most of these NHEJ alleles consisted of deletions (ranging from 1 to 83 nt), which were located primarily on the 3′ side of the gRNA cleavage site and a few other substitutions/insertions. As expected, Nickases induced very few NHEJ mutations (˜0.4%), and the few recovered events consisted predominantly of deletions 3′ to the cut site when elicited by D10A, and more frequently 5′ to the cut site in response to H840A-dependent nicking. For both Nickases, a large fraction (˜20%) of CS alleles remained intact, in contrast to Cas9, which converted or mutated nearly all CS1⁻ alleles, leaving fewer than 1% of them unaltered (FIG. 7D).

Nickase also Sustains Cassette Copying, Albeit Less Efficiently than Cas9

We reported recently that transgenic cassettes referred to as “CopyCatchers” can be successfully copied to a naïve homologous site in Drosophila somatic cells upon targeted DSB at their site of insertion. These elements, placed into an intron, create a loss-of-function (1-o-f) allele by inserting a fluorescent reporter in-frame with the targeted endogenous gene. When combined in cis with an ATG⁻ point mutation, CopyCatchers reveal HDR events by generating mutant phenotypes coinciding with DsRed fluorescent marker expression (22). For example, in w^{ATG- [cc]}/w⁺ females expressing Cas9, DNA cleavage targeting the intact w⁺ allele during development results in w⁻ clonal phenotypes, in which somatic gene conversion leads to production of 1-o-f homozygous w^{[cc]}/w^{[cc]} clones also expressing the DsRed reporter. In w^{ATG- [cc]}/w⁺ females expressing D10A, we consistently observed few small w⁻ eye patches also expressing DsRed. We conclude that the D10A nickase can also mediate somatic copying of a gene cassette, but does so less efficiently than Cas9 (˜5-6 fold reduction).

In aggregate, our observations reveal that both Cas9 and Nickases lead to inter-homolog allelic correction in somatic cells, but that they display significantly different dynamics and efficiencies (Table 1). HTR operates at higher efficiency in response to SSB than to DSB, the latter process competing with the NHEJ repair pathway.

### Cas9- and Nickase-Mediated Allelic Correction in Symmetric Genetic Configurations

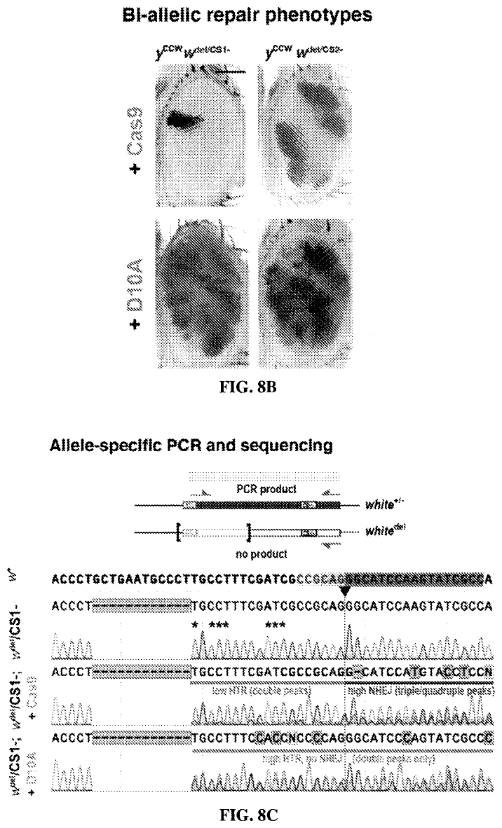

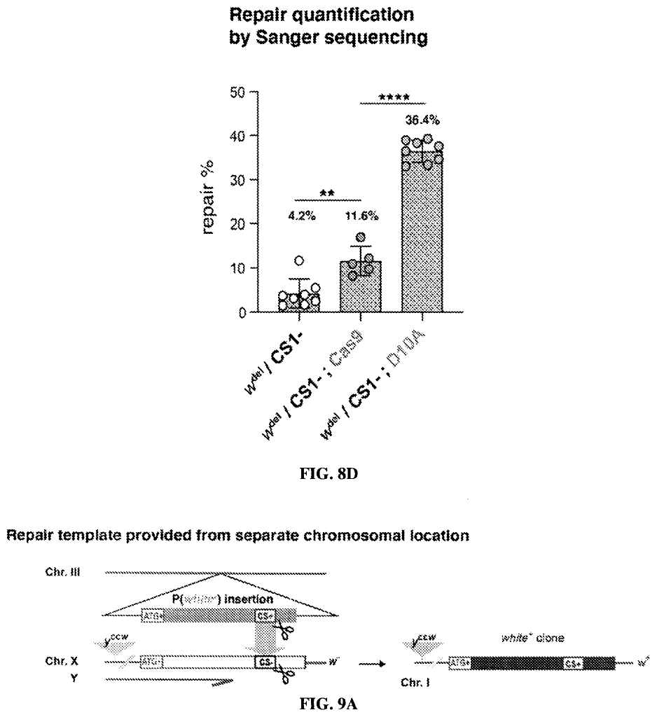

Experiments described above involve allele-specific DNA cleavage, after which cut-resistant sequences are used for directional repair at the homologous site. We tested an alternate “sensitive/sensitive” configuration, for which both alleles of the w locus are subjected to DNA cleavage. For these experiments, we made use of the w¹¹¹⁸ null allele (referred to as w^{del} hereafter), a multi-kb deletion encompassing the first exon and most of the first intron of w (27), yet leaving the w-gRNA recognition site and adjacent sequences intact (FIG. 8A). Trans-heterozygous w^{del}/y^{ccw} w^{ATG+ CS1-} flies produced only rare small red clones in response to Cas9 expression (FIG. 8B, upper left panel), indicating that bi-allelic DSB rarely resolve in successful restoration of functional w⁺ alleles. In contrast, y^{ccw} w^{ATG+ CS1-}/w^{del} females expressing D10A displayed frequent w⁺ clones (FIG. 8B, lower left panel), albeit fewer than observed in comparable flies carrying a cut-resistant CR⁺ allele (FIG. 6A). These results suggest that each allele (w^{CS1-} and w^{del}) can be cut and repaired using the other allele as a template. While the w^{ATG+ CS1-} allele may be repaired using wild-type homologous sequences, leading to a functional w⁺ allele, the w^{del} allele repaired using CS1⁻ sequences always remains non-functional. In either case, both alleles remain sensitive to further cleavage, however additional copying events typically will no longer alter the nature of the repaired allele (functional versus non-functional, FIG. 8A and FIG. 8B). In presence of Cas9, this repeated assault may lead to the ultimate accumulation of NHEJ mutations, and a permanent loss of gene function in most cells. The presence of numerous w⁺ clones in y^{cc} w^{ATG+ CS2-}/w^{del}; Cas9 animals (in which the CS2⁻ allele can be restored to a functional state through NHEJ), but not in equivalent CS1⁻ flies, strongly supports this hypothesis (FIG. 8B).

We further characterized HTR events following bi-allelic cleavage/nicking by performing sequence analysis on control and gene-edited flies. We detected and quantified HTR events by performing selective PCR amplification of the w^{CS1-} allele using a primer annealing to the ATG initiation-codon region (which cannot amplify sequences from the w^{del} allele since it lacks the ATG initiation codon and surrounding sequences) for this selective amplification (FIG. 8C). Sequence analysis of such PCR products revealed only the expected CS1⁻ 12 nt deletion in control w^{del}/w^{ATG+ CS1-} flies (FIG. 8C). In contrast, reads from similar flies expressing D10A displayed double peaks corresponding to overlapping wild-type and CS1⁻ alleles and revealing frequent instances of allelic repair (FIG. 8C, bottom panel). Quantification of such Sanger sequencing data revealed a repair rate of ˜36% (FIG. 8D), which, as expected, is lower than the 52% correction estimate in previous experiments involving a cut-resistant allele and the same D10A source (FIG. 6D). In contrast, w^{del}/w^{ATG+ CS1-}; Cas9 animals exhibited only low levels of correction (˜12%, FIG. 8D) as well as high levels of NHEJ-induced mutations visible as triple and quadruple peaks encompassing the cut site (FIG. 8C middle row). These observations are consistent with our proposed mechanism for the sensitive/sensitive configuration: each allele can be replaced by the other, resulting in a homozygous state for either allele (CS1⁻ or CS⁺). Because these alleles are both cut-sensitive, they remain the target for further cleavage/nicking, unless NHEJ produces cut-resistant (and likely) non-functional alleles. This latter final outcome is frequent with Cas9 but rare for D10A, as supported by both eye phenotypes and sequence analysis (FIG. 8A and FIG. 8D).

### Allelic Correction Does Not Require Long-Range Stable Chromosome Pairing

All repair processes examined above involve copying from an allele present on the homologous chromosome. These events are likely to be facilitated by long-range chromosomal pairing, a phenomenon that is central to crossing over in the germline of multicellular organisms, but that is also prevalent in somatic tissues of dipterans underlying phenomena such as transvection (28-30). Inter-homolog pairing, while rare in typical healthy cells, has been reported in some mammalian cancerous cell lines and can be induced locally by DSBs (21, 31).

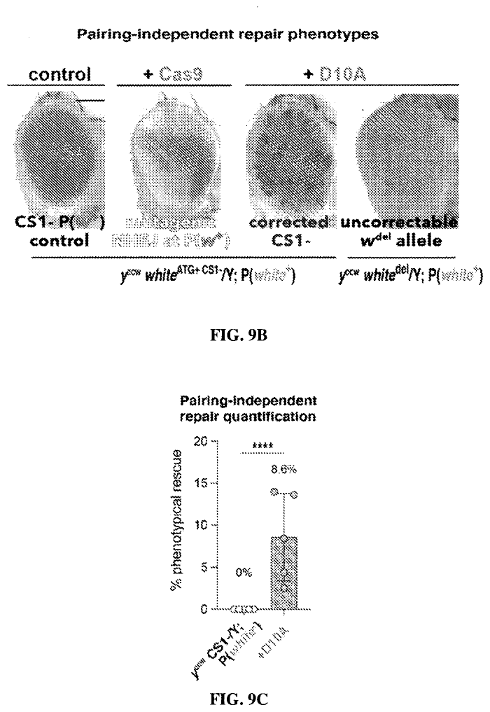

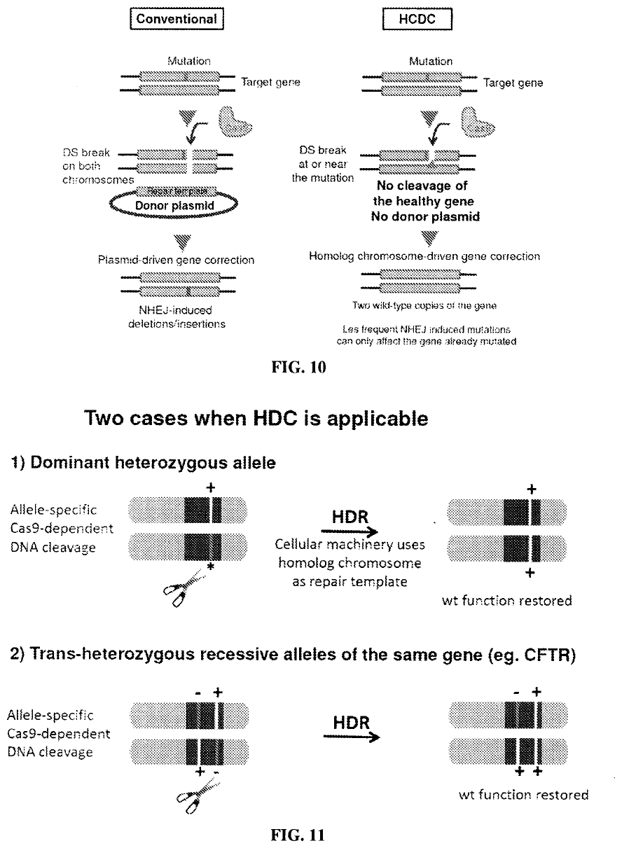

We tested whether allelic repair might also occur in absence of somatic chromosomal pairing in our Drosophila system by using available transgenes carrying a mini-white cDNA that result in variable eye pigmentation phenotypes when inserted at different chromosomal locations. Insertions were selected for the light eye color they produced (when placed in a w^{-/-} background), so that repair events could be distinguished as dark red clones contrasting with yellow or orange backgrounds. y^{ccw} w^{ATG+ CS1-}/Y individuals carrying a P<mini-white⁺> insertion (FIG. 9A) were examined for clonal phenotypes elicited by SSB (D10A) or DSB (Cas9). In the absence of any nuclease, eyes appeared uniformly orange, resulting from expression of the intact mini-white⁺ P-element marker gene. Individuals carrying a source of Cas9 displayed w⁻ clones covered a significant fraction of the eyes (FIG. 9B, left-middle panel), which presumably reflects on the P<mini-white⁺> sequences having been targeted for DSB and repaired through the error-prone NHEJ pathway. When the D10A nickase was assayed in the same genetic context, we instead recovered many small red clones across the eye surface (FIG. 9B, right-middle panel). D10A-induced repair frequencies ranged from ˜2-14% as evaluated by image-based quantification (FIG. 9C). Because we employed the in-frame deletion CS1⁻ allele for these experiments, the observed functional repair can only be attributed to HDR, and not to an alternative mutagenic process affecting the P<mini-white⁺> insertion. Indeed, when the w^{[del]} allele (which cannot be restored to a functional state by allelic correction) was used instead of the CS1⁻ allele (in y^{ccw} w^{[del]}/Y; D10A/; P<mini-white⁺>/+ individuals), no red clones were generated (FIG. 9B, right-most panel).

We also tested two other autosomal P<mini-white⁺> insertions for their ability to serve as template for such pairing-independent correction. We found similar small red clone phenotypes with varying prevalence induced by D10A, but not Cas9, indicating that this form of homolog-independent repair does not depend on a particular genomic location of the repair template. We conclude that allelic correction can occur in a fashion that relies only on sequence homology within the mini-white gene but not on long-range chromosome pairing, a process we refer to as pairing-independent repair.

Cumulatively, these varied genetic and molecular assessments demonstrate that highly efficient somatic allelic conversion at the white locus operates after allele-specific or bi-allelic targeted cleavage or nicking. Importantly, this process is promoted more efficiently by non-mutagenic D10A or H840A nickases than by Cas9, and does not depend strictly on chromosome pairing, although such pairing increases correction frequencies (summarized in Table 1).

### Discussion

In this study, we have developed a versatile genetic system, in which targeted DNA breaks created either by Cas9 or Nickases elicit distinct repair processes, revealed by quantifiable pigmentation phenotypes in Drosophila. We employed a variety of allelic combinations revealing that HDR processes using cut-resistant sequences from the homologous chromosome as repair templates (HTR) are surprisingly efficient in somatic cells. The most striking finding of our study is that two Cas9-derived mutant nucleases, D10A and H840A, which nick rather than cleave target DNA, sustain high rates of somatic allelic conversion (45-65%) that are even greater than those observed with Cas9 (˜30%). As is widely documented in various vertebrate and invertebrate systems, a desirable feature of Nickases is that they lead to far fewer NHEJ-generated mutations (˜1.5%) than Cas9 (24, 32-34). Our allelic correction system is suitable for conducting genetic screens to characterize and optimize HTR as has been previously performed for germline-acting components (35), and to test the influence of other critical parameters, such as transcription and chromatin conformation. In addition, the nature, position and distance of the repaired allele relative to the nuclease cleavage site, introduction of a second gRNA promoting nicking of the opposite strand at a nearby site are also likely to have a strong influence on repair outcomes.

Patterns of Nickase-induced SSB repair appear to be very different from those resulting from Cas9-induced DSB repair. Nickases produced far fewer NHEJ mutations, and initiated HTR at later phases of development than Cas9 as revealed by the smaller sizes and higher numbers of clones. D10A can sustain somatic copying of multi-kb insertions, but does so much less efficiently than Cas9 (the reverse of their activities for allelic repair). In addition, D10A supports only low-level germline copying of cleavage-resistant alleles, while Cas9 does so with great efficiency. These notable differences in Nickase versus Cas9 activity can be interpreted in light of previous knowledge of DSB and SSB repair mechanisms. DSB are repaired either through mutagenic NHEJ or HDR, which involves copying from the sister chromatid (during S and G2 phases) or from the homologous chromosome (during all phases or cell cycle). Both processes will give rise to non-cleavable sequences (NHEJ-induced mutations or copying the cut-resistant allele from the homologous chromosome), such that an end point is reached early after the first few repair cycles, generating large solid w⁺ and w⁻ clones in our system. In contrast, DNA nicks are generally repaired precisely through direct re-ligation (36, 37), thereby restoring an intact cut site amenable to reiterative nicking. Thus, repeated nick-and-repair cycles are likely to take place until conditions favoring HDR arise, wherein copying of cut-resistant sequences from the homologous chromosome terminates this cyclic process. During replication, proximity of a junction between Okasaki fragments on the discontinuously replicating strand could convert nicks into DSB with overhangs. It is also possible that nicks resolve into DSB during transcription by disrupting transcription-dependent repair processes (38). Juxtaposition of different SSB on opposing strands may result in effective DSB with overhangs of varying sizes (36, 39, 40), which may then be amenable to repair by standard or alternate HDR processes.

Inter-homolog repair has generally been assumed to be an uncommon occurrence in somatic cells, in which the NHEJ pathway prevails and operates throughout the cell cycle to repair DNA breaks (41) in contrast to HDR, thought to be restricted to G2 and S phases. Moreover, homolog-based gene conversion events lead to loss-of-heterozygosity (LOH) which can lead to detrimental outcomes such as cancer (42). In contrast, during meiosis in the germline, chromosome pairing between chromosome homologs favors HDR as well as recombination (43), which involves extensive pairing (synapsis) between homologous chromosomes. Such long-range homolog pairing is not typically observed in mammalian somatic cells, although local interhomolog pairing can be induced by DBS (44, 45).

Mechanistic and genetic studies suggest that HDR operating at nicks acts either through the canonical Rad51 and BRCA2 factors, or through an efficient alternative Rad51-independent pathway (32). In this study nicking the transcribed DNA strand led to higher level of repair than nicking the coding strand, as observed in our system (FIG. 7A). Also, increased levels of the RecQ5A helicase have been shown to favor a shift toward this alternate pathway (46). In S. pombe, nick-induced HDR employing the sister chromatid as repair template can be visualized in PNKP⁻ mutants. The absence of PNKP activity prevents direct re-ligation and promotes a Rad51-independent HDR pathway (47). Another example has been documented in birds in which pseudogene-templated gene conversion is essential for generating Ig diversity and is initiated by DNA nicks at the V segment of the light chain locus (34, 48). These established examples of HDR induced at nicks in somatic eukaryotic cells, ranging from yeast to humans suggest that HTR processes rely on broadly conserved repair machineries. Our allelic repair system in Drosophila is sensitive and quantitative, and should be amenable in future studies for screening RNAi and mis-expression lines to potentially identify conserved factors critical for Cas9- or Nickase-induced HTR.

Dipteran insect chromosomes differ from those of mammals by engaging in extensive somatic pairing (21, 31, 49), as illustrated by the phenomenon of transvection, wherein regulatory sequences from one chromosome promote expressing of coding sequences from the homologous chromosome (28-30). Thus, such somatic chromosome pairing is expected to play an essential role in favoring HTR observed in our system over other repair mechanisms. However, we found that HTR can also operate, albeit with reduced efficiency, when the homologous donor DNA is provided from a distinct chromosomal location. Similar pairing-independent gene conversion has been achieved in the insect germline for converting traditional GAL4 into orthogonal QF2 insertions, for which success depends highly on respective chromosomal position of donor and recipient sequences (50). These and our systems may thus serve as valuable genetic models for identifying new pathway components contributing to Nickase-based allelic conversion that are relevant also to mammalian cells.

While most CRISPR-based genome editing strategies typically rely on exogenously provided DNA repair templates, instances of inter-homolog repair have also been reported. For example, Cas9-induced DNA breaks can result in directional copying of a gene cassette onto the homologous chromosome in human HEK293T cells (22) and homozygosity of a cut-resistant allele in mouse embryos when Rad51 is provided in excess (23). Other studies also suggest that SSB-mediated HTR may be achievable in various mammalian systems. Thus, DNA nicks in human HEK293T cells could lead to plasmid-templated HDR in human cells, particularly when two nicks were induced on opposite strands of the targeted sequence at distances ranging from 37-68 nt (51), which can be augmented further by also nicking the exogenous donor DNA template (34, 52, 53) (54, 55).

CRISPR-based gene editing offers great promise for gene therapy. However, numerous reports have raised concerns regarding Cas9-dependent production of large deletions and their potential deleterious off-target activities (56, 57). Nick-induced HTR offers a far less mutagenic and much safer alternative for such therapeutic applications. Although the findings summarized above suggest that inter-homolog HTR at nicks is achievable, it may not be as effective in human cells as we observed in Drosophila. In contrast to dipterans, vertebrates chromosomes are generally thought to remain separated in different chromosomal territories (21), as indicated by numerous studies involving DNA-FISH and Hi-C analysis (58-60). Pairing-independent allelic conversion we observe in Drosophila however, may serve as an excellent model for optimizing allelic correction for both fly and mammalian systems. If the frequency of such events could be increased either by promoting inter-homolog pairing or by optimizing nick-specific repair processes, such strategies could be harnessed to correct numerous dominant or trans-heterozygous disease-causing mutations.

### Material and Methods

### Plasmid Construction

To create the white− Cut-Sensitive alleles and the ATG− mutation described in FIG. 5A and FIG. 5B, three plasmids were constructed to express the 5′-gRNA, the 3′-gRNA, or the ATG-gRNA. Annealed oligos were inserted into the PCFD3 vector after digestion with Bbs1 as described on www.crisprflydesign.org/plasmids/. Sequences of the three pair of oligos were:

5′-gRNA:
(SEQ ID NO: 5)
Forward = GTCGGAAAGGCAAGGGCATTCAGCA
(SEQ ID NO: 6)
Reverse = AAACTGCTGAATGCCCTTGCCTTTC
3′-gRNA:
(SEQ ID NO: 7)
Forward = GTCGGCCATTGAGCAGTCGCATCC
(SEQ ID NO: 8)
Reverse = AAACGGATGCGACTGCTCAATGGC
ATG-gRNA
(SEQ ID NO: 9)
Forward = GTCGAGTGTGAAAAATCCCGGCAAT
(SEQ ID NO: 10)
Reverse = AAACATTGCCGGGATTTTTCACACT

Microinjection of gRNA Constructs and Establishment of White⁻ Lines

Plasmids were prepared using the Qiagen Plasmid Midi kit (#12191) and sequence-checked. Each gRNA construct was co-injected with a transient source of Cas9 (pAct-Cas9, Addgene plasmid #62209) by Rainbow Transgenics. Injection mixes were assembled with each gRNA plasmid (final concentration: 500 ng/μl) and pAct-Cas9 (final concentration: 500 ng/μl) in a volume of 50 μl. Injection mixes for all gRNA constructs were injected into an Oregon-R (white⁺) stock (BDSC #2376). White− mutant males were selected in F1 progeny to establish isogenic lines and specific alterations were determined by sequencing.

### Drosophila Genetics

The y^{ccw} insertion was described in S2 of (12). Flies carrying the y^{ccw} insertion identified by Dsred fluorescence in eyes was recombined with The ATG− mutation and associated with the CR+ allele using Cas9-mediated allelic conversion to generate the y^{ccw} ATG− CR+ donor (cut-resistant) line (DsRed⁺ white⁻) used throughout this study. Cas9 was expressed from the third chromosome insertion PBac{vas-Cas9}VK00027 (BL #51324, marked with 3XP3-GFP), which expresses Cas9 in the germline and somatically. D10A was expressed from the second chromosome insertion (y1 w1118 P(3xP3-EGFP, vasa-cas9D10A)attP40A, a gift from Avi Rodal (Brandeis University). Lines were established combining CS− alleles and each nuclease, males from such lines were crossed to females from y^{ccw} ATG− CR donor lines to produce experimental animals. For comparing the activity of the different nucleases and deep-sequencing analysis (FIG. 7C), three lines were designed in which vasaCas9, vasaD10A, or vasaH840A sequences associated with the DsRed marker were inserted at the same location in the yellow gene (26). For pairing-independent HDR (FIG. 9B), the following autosomal P(white+) insertions producing orange eye phenotypes were used: BL #1799 (P{GAL4-Hsp70.PB}89-2-1, chr. 3), BL #2077 (P{GAL4-Hsp70.PB}2, chr. 2), BL #1822 (P{GAL4-Hsp70.PB}31-1, chr. 3).

### Sequence Analysis of Allelic Correction

To establish sequences and correction percentages shown in FIG. 6B, genomic DNA was extracted from individual flies of relevant genotypes (yccw ATG− CR+/y+ ATG+ CS1−; +/− vasaCas9/vasaD10A). PCR reactions were assembled using the Q5 Hotstart master mix (NEB #M0494S) with the following primers:

(SEQ ID NO: 11)
Forward: CTGCTCATTGCACTTATCTACAAG
(SEQ ID NO: 12)
Reverse: GCAAATTAAAATGTTACTCGCATCTC

2.2 Kb PCR products were purified prior to Sanger sequencing with internal primers:

(SEQ ID NO: 13)
Forward: GCTGGTCAACCGGACACGCGG (for the CS1-
allele)
(SEQ ID NO: 14)
Reverse: CTCGCTGCCGATAGGTCAGATGTCG (for the CS2-
allele).

To evaluate correction percentages, seven peaks (marked with the * symbol in FIG. 6C) located in the CS1_12 nt deletion were chosen for consistent low distortion (similar peak heights for the CS1− and CR+ alleles in heterozygous control animals). For each peak, correction percentage was calculated using the formula: [pv(CR+)−pv(CS1−)]*100/[pv(CR+)+pv(CS1−)], where “pv” refers to the peak value of the indicated allele (CR+ or CS1−) read in the SNAPgene program. Seven “low distortion” peaks located between the cut site and the CS2_1 nt insertion were chosen for quantifying HTR of the CS2_allele.

In FIG. 7C, allele-specific PCR was performed using a forward primer specific for the wt ATG+ allele to generate a 3.7 Kb product:

(SEQ ID NO: 15)
Forward: GTGTGAAAAATCCCGGCAATGG
(SEQ ID NO: 16)
Reverse: AGGGAGCCGATAAAGAGGTCATCC

PCR products were sequenced with the same internal primer as in FIG. 6C. Allelic correction percentages were calculated as follows: pv(CR+)*100/[pv(CR+)+pv(CS1−)], which takes in account the fact that only the receiver allele (ATG+) is read in these samples.

### Allelic Repair Quantification by Image Analysis

Eye images were acquired on a ZEISS Axio Zoom.V16 microscope at 112× magnification with an Axiocam 506 color camera, using the Zen pro 2012 software. For each eye, Z-stacks of 14-20 images at ˜10 um intervals at 20 ms exposure for the GFP channel, 50 ms for the DsRed channel, and X ms for brightfield images were acquired. Focus stacking was performed using Helicon Focus 7.5.4 software and saved in tif format.

### Image-Based Quantifications

The tif Images were opened in ImageJ, brightness and contrast were adjusted using the “auto” tool (Image>adjust>bright/cont>auto). Images were converted to black and white using the Type function (8bit) in the image menu. Using the freehand tool, the total area of the eye was encircled. Black clones were identified using the threshold function (Image>adjust>threshold). Total area of each pigmented clones was evaluated using the “Analyze particles” function (Analyze→analyze particles), while the total area of the eye was calculated using the “area” function (Analyze>set measurements>Area). The percentage of total area of all particles (areas representing repair) relative to the total area of the eye, was calculated for each eye and plotted in Prism 9.2.0.

### Amplicon-Based Deep Sequencing

Genomic DNA was extracted from group of 10 flies or single flies of each genotype. Sequences around the gRNA cleavage site were amplified by PCR using primers specifically designed for deep-sequencing, with 5′ tails complementary to the Illumina partial adaptors (5′-ACACTCTTTCCCTACACGACGCTCTTCCGATCT-3′ (SEQ ID NO: 3) for forward primers and 5′-GACTGGAGTTCAGACGTGTGCTCTTCCGATCT-3′ (SEQ ID NO: 4) for reverse primers). Two sets of primers were designed to avoid primer-specific artifacts:

Set1:
(SEQ ID NO: 17)
ACACTCTTTCCCTACACGACGCTCTTCCGATCTccaatttgaaactcagt
ttgc
(SEQ ID NO: 18)
GACTGGAGTTCAGACGTGTGCTCTTCCGATCTgtcatcctgctggacata
g
Set 2:
(SEQ ID NO: 19)
ACACTCTTTCCCTACACGACGCTCTTCCGATCTgcgcccaggaaacattt
gctcaag
(SEQ ID NO: 20)
GACTGGAGTTCAGACGTGTGCTCTTCCGATCTcgctgccgataggtcaga
tgtcg

PCR products were gel purified and 20 ng/ul of each sample was sent for Illumina paired-end 150-500 bp Amplicon-based deep sequencing. All reads were analyzed using manual CRISPResso2 command lines (crispresso.pinellolab.partners.org/submission), with wild-type white gene used as reference.

To establish the % of allelic correction, we first established a normalization factor (NF) from the control group, which takes in account the rate of PCR errors found in each experiment. NF=100/(% CR+% CS1+% PCR-rec1+% PCR-rec2). This factor brings the total percentage of events to 100% and eliminates the contribution of PCR errors. % repair is then calculated as: NF*(% CRexp.−% CRcont.)*2.

### Ethical Conduct of Research

We have complied with all relevant ethical regulations for animal testing and research and conformed to the UCSD institutionally approved biological use authorization protocol (BUA #311).

### Statistical Analysis

All the experimental data presented in this study are from at least three independent experiments. Statistical data was analyzed and plotted using GraphPad Prism 9.2.0 by two-tailed t-test. The standard deviation (SD) is represented by error bars in the Bar graphs centred around the mean, and to confirm significance, p-values were calculated.

### REFERENCES (Example 2)

1. P. Horvath et al., Diversity, activity, and evolution of CRISPR loci in Streptococcus thermophilus. Journal of bacteriology 190, 1401-1412 (2008).
2. M. Jinek et al., A programmable dual-RNA-guided DNA endonuclease in adaptive bacterial immunity. science 337, 816-821 (2012).
3. M. Adli, The CRISPR tool kit for genome editing and beyond. Nature communications 9, 1-13 (2018).
4. E. Bier, M. M. Harrison, K. M. O'Connor-Giles, J. Wildonger, Advances in engineering the fly genome with the CRISPR-Cas system. Genetics 208, 1-18 (2018).
5. A. V. Anzalone, L. W. Koblan, D. R. Liu, Genome editing with CRISPR-Cas nucleases, base editors, transposases and prime editors. Nature biotechnology 38, 824-844 (2020).
6. H. Li et al., Applications of genome editing technology in the targeted therapy of human diseases: mechanisms, advances and prospects. Signal transduction and targeted therapy 5, 1-23 (2020).
7. R. Scully, A. Panday, R. Elango, N. A. Willis, DNA double-strand break repair-pathway choice in somatic mammalian cells. Nature reviews Molecular cell biology 20, 698-714 (2019).
8. P. D. Hsu, E. S. Lander, F. Zhang, Development and applications of CRISPR-Cas9 for genome engineering. Cell 157, 1262-1278 (2014).
9. M. Bibikova et al., Stimulation of homologous recombination through targeted cleavage by chimeric nucleases. Molecular and cellular biology 21, 289-297 (2001).
10. M. Bibikova, M. Golic, K. G. Golic, D. Carroll, Targeted chromosomal cleavage and mutagenesis in Drosophila using zinc-finger nucleases. Genetics 161, 1169-1175 (2002).
11. V. M. Gantz et al., Highly efficient Cas9-mediated gene drive for population modification of the malaria vector mosquito Anopheles stephensi. Proceedings of the National Academy of Sciences 112, E6736-E6743 (2015).
12. V. L. Del Amo et al., A transcomplementing gene drive provides a flexible platform for laboratory investigation and potential field deployment. Nature communications 11, 1-12 (2020).
13. G. Terradas et al., Inherently confinable split-drive systems in Drosophila. Nature communications 12, 1-12 (2021).
14. V. M. Gantz, E. Bier, The mutagenic chain reaction: a method for converting heterozygous to homozygous mutations. Science 348, 442-444 (2015).
15. H. A. Grunwald et al., Super-Mendelian inheritance mediated by CRISPR-Cas9 in the female mouse germline. Nature 566, 105-109 (2019).
16. V. M. Gantz, E. Bier, The dawn of active genetics. Bioessays 38, 50-63 (2016).
17. A. Guichard et al., Efficient allelic-drive in Drosophila. Nature communications 10, 1-10 (2019).
18. J. Li et al., Genome-block expression-assisted association studies discover malaria resistance genes in Anopheles gambiae. Proceedings of the National Academy of Sciences 110, 20675-20680 (2013).
19. L. Grigoraki et al., CRISPR/Cas9 modified An. gambiae carrying kdr mutation L1014F functionally validate its contribution in insecticide resistance and interaction with metabolic enzymes. bioRxiv, (2021).
20. Kaduskar, Reversing insecticide resistance with allelic-drive in Drosophila melanogaster. Nature communications, “In Press”, (2022).
21. E. F. Joyce, J. Erceg, C.-t. Wu, Pairing and anti-pairing: a balancing act in the diploid genome. Current opinion in genetics & development 37, 119-128 (2016).
22. Z. Li et al., CopyCatchers are versatile active genetic elements that detect and quantify inter-homolog somatic gene conversion. Nature communications 12, 1-12 (2021).
23. J. J. Wilde et al., Efficient embryonic homozygous gene conversion via RAD51-enhanced interhomolog repair. Cell, (2021).
24. A. E. Trevino, F. Zhang, Genome editing using Cas9 nickases. Methods in enzymology 546, 161-174 (2014).
25. F. Port, H.-M. Chen, T. Lee, S. L. Bullock, Optimized CRISPR/Cas tools for efficient germline and somatic genome engineering in Drosophila. Proceedings of the National Academy of Sciences 111, E2967-E2976 (2014).
26. V. Lopez del Amo, V. Gantz, CRISPR gene-drive systems based on Cas9 nickases promote super-Mendelian inheritance in Drosophila. (2021).
27. A. Platts et al., Massively parallel resequencing of the isogenic Drosophila melanogaster strain w1118; iso-2; iso-3 identifies hotspots for mutations in sensory perception genes. Fly 3, 192-204 (2009).
28. J. A. Abed et al., Highly structured homolog pairing reflects functional organization of the Drosophila genome. Nature communications 10, 1-14 (2019).
29. Q. Szabo et al., TADs are 3D structural units of higher-order chromosome organization in Drosophila. Science advances 4, eaar8082 (2018).
30. J. Erceg et al., The genome-wide multi-layered architecture of chromosome pairing in early Drosophila embryos. Nature communications 10, 1-13 (2019).
31. M. S. Apte, V. H. Meller, Homologue pairing in flies and mammals: gene regulation when two are involved. Genetics research international 2012, (2012).
32. L. Davis, N. Maizels, Homology-directed repair of DNA nicks via pathways distinct from canonical double-strand break repair. Proceedings of the National Academy of Sciences 111, E924-E932 (2014).
33. L. E. Vriend et al., Distinct genetic control of homologous recombination repair of Cas9-induced double-strand breaks, nicks and paired nicks. Nucleic acids research 44, 5204-5217 (2016).
34. N. Maizels, L. Davis, Initiation of homologous recombination at DNA nicks. Nucleic acids research 46, 6962-6973 (2018).
35. D. S. Wei, Y. S. Rong, A genetic screen for DNA double-strand break repair mutations in Drosophila. Genetics 177, 63-77 (2007).
36. K. W. Caldecott, Single-strand break repair and genetic disease. Nature Reviews Genetics 9, 619-631 (2008).
37. R. Abbotts, D. M. Wilson III, Coordination of DNA single strand break repair. Free Radical Biology and Medicine 107, 228-244 (2017).
38. G. Kokic, F. R. Wagner, A. Chernev, H. Urlaub, P. Cramer, Structural basis of human transcription—DNA repair coupling. Nature, 1-5 (2021).
39. A. Kuzminov, Single-strand interruptions in replicating chromosomes cause double-strand breaks. Proceedings of the National Academy of Sciences 98, 8241-8246 (2001).
40. L. H. Thompson, K. W. Brookman, N. J. Jones, S. A. Allen, A. V. Carrano, Molecular cloning of the human XRCC1 gene, which corrects defective DNA strand break repair and sister chromatid exchange. Molecular and cellular biology 10, 6160-6171 (1990).
41. K. Siudeja, A. J. Bardin, Somatic recombination in adult tissues: What is there to learn? Fly 11, 121-128 (2017).
42. C. A. Nichols et al., Loss of heterozygosity of essential genes represents a widespread class of potential cancer vulnerabilities. Nature communications 11, 1-14 (2020).
43. Y. S. Rong, K. G. Golic, Gene targeting by homologous recombination in Drosophila. science 288, 2013-2018 (2000).
44. M. Gandhi et al., Homologous chromosomes make contact at the sites of double-strand breaks in genes in somatic GO/G1-phase human cells. Proceedings of the National Academy of Sciences 109, 9454-9459 (2012).
45. M. Gandhi, V. N. Evdokimova, K. T. Cuenco, C. J. Bakkenist, Y. E. Nikiforov, Homologous chromosomes move and rapidly initiate contact at the sites of double-strand breaks in genes in Go-phase human cells. Cell cycle 12, 547-552 (2013).
46. H. C. Olson et al., Increased levels of RECQS shift DNA repair from canonical to alternative pathways. Nucleic acids research 46, 9496-9509 (2018).
47. A. Sanchez, M. C. Gadaleta, O. Limbo, P. Russell, Lingering single-strand breaks trigger Rad51-independent homology-directed repair of collapsed replication forks in the polynucleotide kinase/phosphatase mutant of fission yeast. PLoS genetics 13, e1007013 (2017).
48. H. Arakawa, J. M. Buerstedde, Immunoglobulin gene conversion: insights from bursal B cells and the DT40 cell line. Developmental dynamics: an official publication of the American Association of Anatomists 229, 458-464 (2004).
49. B. D. McKee, Homologous pairing and chromosome dynamics in meiosis and mitosis. Biochimica et Biophysica Acta (BBA)-Gene Structure and Expression 1677, 165-180 (2004).
50. C.-C. Lin, C. J. Potter, Editing transgenic DNA components by inducible gene replacement in Drosophila melanogaster. Genetics 203, 1613-1628 (2016).
51. F. A. Ran et al., Double nicking by RNA-guided CRISPR Cas9 for enhanced genome editing specificity. Cell 154, 1380-1389 (2013).
52. A. M. Smith et al., Generation of a nicking enzyme that stimulates site-specific gene conversion from the I-Anil LAGLIDADG (SEQ ID NO: 21) homing endonuclease. Proceedings of the National Academy of Sciences 106, 5099-5104 (2009).
53. L. Davis, N. Maizels, DNA nicks promote efficient and safe targeted gene correction. PloS one 6, e23981 (2011).
54. K. Nakajima et al., Precise and efficient nucleotide substitution near genomic nick via noncanonical homology-directed repair. Genome research 28, 223-230 (2018).
55. T. Hyodo et al., Tandem paired nicking promotes precise genome editing with scarce interference by p53. Cell reports 30, 1195-1207. e1197 (2020).
56. I. Weisheit et al., Detection of deleterious on-target effects after HDR-mediated CRISPR editing. Cell Reports 31, 107689 (2020).
57. G. Cullot et al., CRISPR-Cas9 genome editing induces megabase-scale chromosomal truncations. Nature communications 10, 1-14 (2019).
58. C. Heride et al., Distance between homologous chromosomes results from chromosome positioning constraints. Journal of Cell Science 123, 4063-4075 (2010).
59. S. Selvaraj, J. R Dixon, V. Bansal, B. Ren, Whole-genome haplotype reconstruction using proximity-ligation and shotgun sequencing. Nature Biotechnology 31, 1111-1118 (2013).
60. Suhas S. P. Rao et al., A 3D Map of the Human Genome at Kilobase Resolution Reveals Principles of Chromatin Looping. Cell 159, 1665-1680 (2014).

What is claimed is:

1. A homologous chromosome template repair (HTR) system, comprising:
a gene editing system configured to:
a) cut a mutant allele of a cell at or near a mutation in the mutant allele but not cut a corresponding homologous allele without the mutation; and
b) allow the corresponding homologous allele to act as a template for homology directed repair of the mutant allele, thereby repairing the mutation in the mutant allele.

2. The HTR system of claim 1, wherein the gene editing system comprises a guide RNA and an endonuclease enzyme.

3. The HTR system of claim 2, wherein the endonuclease is a Cas9 or a Cas9 variant selected from D10A and H840A.

4. The HTR system of claim 1, wherein the gene editing system is configured to perform a single-stranded cut of the mutant allele.

5. The HTR system of claim 4, wherein the single-stranded cut is in the encoding strand of the mutant allele.

6. The HTR system of claim 4, wherein the single-stranded cut is in the template strand of the mutant allele.

7. The HTR system of claim 1, wherein the mutation in the mutant allele:
a) creates an endonuclease recognition site in the mutant allele;
b) is near an endonuclease recognition site present on both the mutant allele and the homologous allele; or
c) is near a polymorphic site near an endonuclease recognition site.

8. The HTR system of claim 1, wherein the cell is a mammalian cell or an insect cell.

9. The HTR system of claim 1, wherein the cell is a somatic cell of a multicellular organism.

10. The HTR system of claim 1, wherein the gene editing system does not comprise an exogenous repair template.

11. A vector encoding the HTR system of claim 1.

12. A method of performing a homologous chromosome template repair (HTR) in a cell, comprising:
a) contacting a mutant allele with a gene editing system configured to cut the mutant allele at or near a mutation in the mutant allele but not to cut a homologous allele without the mutation;
b) cutting the mutant allele at or near the mutation in the mutant allele;
c) using the homologous allele as a template for homology directed repair (HDR); and
d) repairing the mutation in the mutant allele.

13. The method of claim 12, wherein the gene editing system comprises a guide RNA and an endonuclease enzyme.

14. The method of claim 13, wherein the endonuclease is selected from a meganuclease, a Transcription Activator Like Effector Nucleases (TALEN), a Zinc-Finger Nucleases (ZFN), and a Clustered Regularly Interspaced Short Palindromic Repeats (CRISPR)-associated system (Cas), and derivatives thereof.

15. The method of claim 13, wherein the endonuclease is a Cas9 or a Cas9 variant selected from D10A and H840A.

16. The method of claim 12, wherein the gene editing system is configured to perform a single-stranded cut of the mutant allele.

17. The method of claim 16, wherein the single-stranded cut is in the encoding strand of the mutant allele.

18. The method of claim 16, wherein the single-stranded cut is in the template strand of the mutant allele.

19. The method of claim 12, comprising introducing the gene editing system or a vector encoding the gene editing system into the cell.

20. The method of claim 12, wherein the mutation in the mutant allele:
a) creates an endonuclease recognition site in the mutant allele;
b) is near an endonuclease recognition site present on both the mutant allele and the homologous allele; or
c) is near a polymorphic site near an endonuclease recognition site.

21. The method of claim 12, wherein the cell is a mammalian cell or an insect cell.

22. The method of claim 12, wherein the cell is a somatic cell of a multicellular organism.

23. An engineered cell for investigating homologous chromosome directed repair, comprising:
a) a first allele which does not express an encoded gene;
b) a second allele homologous to the first allele, wherein the second allele comprises a mutation relative to the first allele; and
c) a guide RNA configured to recruit an endonuclease enzyme to facilitate a cut in the second allele at or near the mutation but not cut the first allele;
wherein the system is configured such that homology directed repair (HDR) of the second allele with the first allele as a template after a cut by the endonuclease enzyme results in the second allele encoding the encoded gene.

24. The engineered cell of claim 23, wherein the encoded gene comprises a reporter gene.

25. The engineered cell of claim 23, wherein the encoded gene comprises a native gene of the first allele.

26. The engineered cell of claim 23, wherein the HDR results in a change in phenotype in the organism relative to the organism with the mutation.

27. The engineered cell of claim 23, wherein the first allele is modified to not express the encoded gene by a mutation of or a deletion of a start codon.

28. The engineered cell of claim 23, wherein the guide RNA is encoded in the first allele.

29. The engineered cell of claim 23, wherein the endonuclease is a Cas9 or a derivative thereof.

30. The engineered cell of claim 23, wherein the endonuclease is configured to perform a single-stranded cut of the second allele.
